(19) United States
(12) Patent Application Publication
(10) Pub. No.: US 2004/0023807 A1
(43) Pub. Date: Feb. 5, 2004

(54) 3-Arylisothiazoles and their use as herbicides

(76) Inventors: Ingo Sagasser, Dannstadt-Schauernheim (DE); Olaf Menke, Altleiningen (DE); Michael Rack, Heidelberg (DE); Gerhard Hamprecht, Weinheim (DE); Michael Puhl, Lampertheim (DE); Robert Reinhard, Ludwigshafen (DE); Matthias Witschel, Bad Durkheim (DE); Cyrill Zagar, Ludwigshafen (DE); Helmut Walter, Obrigheim (DE); Karl-Otto Westphalen, Speyer (DE)

Correspondence Address: KEIL & WEINKAUF, 1350 CONNECTICUT AVENUE, N.W., WASHINGTON, DC, 20036, US

(21) Appl. No.: 10/276,226

(22) PCT Filed: May 14, 2001
(86) PCT No.: PCT/EP01/05457

(30) Foreign Application Priority Data
May 15, 2000 (DE) 100 23 770.3

(51) Int. Cl. A01N 43/40; A01N 43/80; C07D 417/04; C07D 275/02

(52) U.S. Cl. 504/252; 504/269; 546/271.1; 548/206

(57) ABSTRACT

3-Arylisothiazoles of the formula I

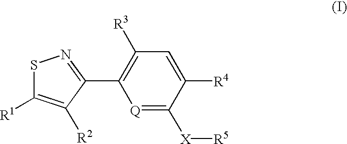

in which the variables X, Q, R¹, R², R³, R⁴, R⁵ are as defined in claim 1, and salts thereof, and their use for controlling harmful plants, are described.

The present invention relates to 3-arylisothiazoles and to their agriculturally useful salts and to their use as herbicides, desiccants or defoliants.

3-Phenylisothiazoles having an unsubstituted phenyl ring have been described by various authors. Thus, L. B. Mylari et al. describe, in J. Med. Chem. 35(3) (1992), 457-465, the use of 5-chloromethylisothiazole as aldose reductase inhibitor. In Tetrahedron 41 (1985), 1885-1892, 3-phenyl-5-methylthioisothiazole is described in connection with the reaction of isothiazolium salts. In Synthesis 4 (1987), 349-353, M. Ishida et al. describe the preparation of 3-phenyl-5-alkylthioisothiazoles starting from tosyl isothiocyanate. 5-Ethoxy- and 5-methylthio-4-cyano-3-phenyl-isothiazole are disclosed, for example, in Tetrahedron 40 (1984), 381-384, and Aust. J. Chem. 42 (1989), 1291-1306.

A large number of herbicidally active compounds having 5-membered heteroaromatic partial structures have been described in the prior art, for example in EP-A 18 080, EP-A 18 497, EP-A 29 171, EP-A 49 760, EP-A 81 730, 38, EP-A 709 380, DE-A 30 18 075, DE-A 30 38 636, DE-A 29 14 003, DE-A 39 29 673, DE-A 42 29 193 and DE-A 195 30 767.

JP-A 63233 982 describes herbicidally active isothiazole-4-sulfonamides substituted by a 6-membered hetaryl group or a 6-membered hetaryl group. WO 97/38987, WO 97/38988 and WO 97/38996 describe highly active herbicides having a benzoylisothiazole structure.

Some of the herbicides having a 5-membered heterocycle which are known from the prior art are unsatisfactory with respect to their activity and/or selectivity for harmful plants. Moreover, there is a constant need for providing novel herbicidally active substances to avoid a possible formation of resistance to known herbicides.

It is an object of the present invention to provide novel herbicides which allow better control of the harmful plants than those of the prior art. Advantageously, the novel herbicides should be highly active against harmful plants. Moreover, it is desirable that they are compatible with crop plants.

We have found that this object is achieved by 3-arylisothiazoles which, in the 5-position of the isothiazole ring, have a substituent selected from the group consisting of C₁-C₄-haloalkyl, C₁-C₄-alkoxy, C₁-C₄-haloalkoxy, C₁-C₄-alkylthio, C₁-C₄-haloalkylthio, C₁-C₄-alkylsulfinyl, C₁-C₄-haloalkylsulfinyl, C₁₋C₄-alkylsulfonyl, C₁-C₄-haloalkylsulfonyl, C₁-C₄-alkylsulfonyloxy, C₁-C₄-haloalkylsulfonyloxy, and which carry a phenyl ring in the 3-position, which phenyl ring is at least monosubstituted and/or has a fused-on 5- or 6-membered heterocycle.

Accordingly, the invention relates to 3-arylisothiazoles of the formula I

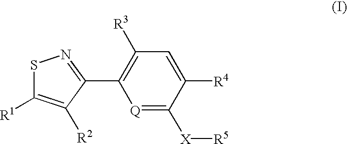

in which the variables X, Q, R¹, R², R³, R⁴, R⁵ are as defined below:

X is a chemical bond or a methylene, 1,2-ethylene, propane-1,3-diyl, ethene-1,2-diyl or ethyne-1,2-diyl chain or an oxymethylene or thiamethylene chain which is attached to the phenyl ring via the heteroatom, where all chains may be unsubstituted or may carry one or two substituents, in each case selected from the group consisting of cyano, carboxyl, halogen, C₁-C₄-alkyl, C₁-C₄-haloalkyl, C₁-C₄-alkoxy, (C₁-C₄-alkoxy)carbonyl, di(C₁-C₄-alkyl)amino and phenyl;

R¹ is C₁-C₄-haloalkyl, C₁-C₄-alkoxy, C₁-C₄-haloalkoxy, C₁-C₄-alkylthio, C₁-C₄-haloalkylthio, C₁-C₄-alkylsulfinyl, C₁-C₄-haloalkylsulfinyl, C₁-C₄-alkylsulfonyl, C₁-C₄-haloalkylsulfonyl, C₁-C₄-alkylsulfonyloxy or C₁-C₄-haloalkylsulfonyloxy;

R² is hydrogen, halogen, amino, cyano, nitro, C₁-C₄-alkyl or C₁-C₄-haloalkyl;

R³ is hydrogen or halogen;

R⁴ is hydrogen, cyano, nitro, halogen, C₁-C₄-alkyl, C₁-C₄-haloalkyl, C₁-C₄-alkoxy or C₁-C₄-haloalkoxy;

R⁵ is hydrogen, nitro, cyano, halogen, halosulfonyl, —O—Y—R⁷, —O—CO—Y—R⁷, —N(Y—R⁷)(Z—R⁸), —N(Y—R⁷)—SO₂—Z—R⁸, —N(SO₂—Y—R⁷)(SO₂—Z—R⁸), —N(Y—R⁷)—CO—Z—R⁸, —N(Y—R⁷)(O—Z—R⁸), —S—Y—R⁷, —SO—Z—R⁷, —SO₂—Y—R⁷, —SO₂—O—Y—R⁷, —SO₂—N(Y—R⁷)(Z—R⁸), —CO—Y—R⁷, —C(═NOR⁹)—Y—R⁷, —C(═NOR⁹)—O—Y—R⁷, —CO—O—Y—R⁷, —CO—S—Y—R⁷, —CO—N(Y—R⁷)(Z—R⁸), —CO—N(Y—R⁷)(O—Z—R⁸) or —PO(O—Y—R⁷)₂;

Q is nitrogen or a group C—R⁶ in which R⁶ is hydrogen; or

R⁴ and X—R⁵ or X—R⁵ and R⁶ are a 3- or 4-membered chain whose chain members may, in addition to carbon, include 1, 2 or 3 heteroatoms selected from the group consisting of nitrogen, oxygen and sulfur and which may be unsubstituted or may for its part carry one, two or three substituents, and whose members may also include one or two not adjacent carbonyl, thiocarbonyl or sulfonyl groups,

where at least one of the variables R³, R⁴ and/or the group X—R⁵ is different from hydrogen and where the variables Y, Z, R⁷, R⁸ and R⁹ are as defined below:

Y, Z independently of one another are: a chemical bond, a methylene or ethylene group which may be unsubstituted or may carry one or two substituents, in each case selected from the group consisting of carboxyl, C₁-C₄-alkyl, C₁-C₄-haloalkyl, (C₁-C₄-alkoxy)carbonyl and phenyl;

R⁷, R⁸ independently of one another are: hydrogen, C₁-C₆-haloalkyl, C₁-C₄-alkoxy-C₁-C₄-alkyl, C₂-C₆-alkenyl, C₂-C₆-haloalkenyl, C₂-C₆-alkynyl, C₂-C₆-haloalkynyl, —CH(R¹⁰)(R¹¹), —C(R¹⁰)(R¹¹)—NO₂, —C(R¹⁰)(R¹¹)—CN, —C(R¹⁰)(R¹¹)-halogen, —C(R¹⁰)(R¹¹)—OR¹², —C(R¹⁰)(R¹¹)—N(R¹²)R¹³, —C(R¹⁰)(R¹¹)—N(R¹²)—OR¹³, —C(R¹⁰)(R¹¹)—SR¹², —C(R¹⁰)(R¹¹)—SO—R¹², —C(R¹⁰)(R¹¹)—SO₂—R¹², —C(R¹⁰)(R¹¹)—SO₂—OR¹², —C(R¹⁰)(R¹¹)—SO₂—N(R¹²)R¹³, —C(R¹⁰)(R¹¹)—CO—R¹², —C(R¹⁰)(R¹¹)—C(═NOR¹⁴)—R¹², —C(R¹⁰)(R¹¹)—CO—OR¹², —C(R¹⁰)(R¹¹)—CO—SR¹², —C(R¹⁰)(R¹¹)—CO—N(R¹²)R¹³, —C(R¹⁰)(R¹¹)—CO—N(R¹²)—OR¹³, —C(R¹⁰)(R¹¹)—PO(OR¹²)₂, C₃-C₈-cycloalkyl which may contain a carbonyl or thiocarbonyl ring member, phenyl or 3-, 4-, 5-, 6- or 7-membered heterocyclyl which may contain a carbonyl or thiocarbonyl ring member, where each cycloalkyl, the phenyl and each heterocyclyl ring may be unsubstituted or may carry one, two, three or four substituents, in each case selected from the group consisting of cyano, nitro, amino, hydroxyl, carboxyl, halogen, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₁-C₄-alkylsulfonyl, C₁-C₄-haloalkylsulfonyl, (C₁-C₄-alkyl)carbonyl, (C₁-C₄-haloalkyl)carbonyl, (C₁-C₄-alkyl)carbonyloxy, (C₁-C₄-haloalkyl)carbonyloxy, (C₁-C₄-alkoxy)carbonyl and di(C₁-C₄-alkyl)amino;

R⁹ is hydrogen, C₁-C₆-alkyl, C₁-C₆-haloalkyl, C₁-C₄-alkoxycarbonyl-C₁-C₄-alkyl, C₂-C₆-alkenyl, C₂-C₆-haloalkenyl, C₂-C₆-alkynyl, C₂-C₆-haloalkynyl, C₃-C₈-cycloalkyl, phenyl or phenyl-C₁-C₄-alkyl;

where the variables R¹⁰ to R¹⁴ are as defined below:

R¹⁰, R¹¹ independently of one another are hydrogen, C₁-C₄-alkyl, C₁-C₄-alkoxy-C₁-C₄-alkyl, C₁-C₄-alkylthio-C₁-C₄-alkyl, (C₁-C₄-alkoxy)carbonyl-C₁-C₄-alkyl or phenyl-C₁-C₄-alkyl, where the phenyl ring may be unsubstituted or may carry one to three substituents, in each case selected from the group consisting of cyano, nitro, carboxyl, halogen, C₁-C₄-alkyl, C₁-C₄-haloalkyl and (C₁-C₄-alkoxy)carbonyl;

R¹², R¹³ independently of one another are hydrogen, C₁-C₆-alkyl, C₁-C₆-haloalkyl, C₁-C₄-alkoxy-C₁-C₄-alkyl, C₂-C₆-alkenyl, C₂-C₆-haloalkenyl, C₂-C₆-alkynyl, C₂-C₆-haloalkynyl, C₃-C₈-cycloalkyl, C₃-C₈-cycloalkyl-C₁-C₄-alkyl, phenyl, phenyl-C₁-C₄-alkyl, 3- to 7-membered heterocyclyl or heterocyclyl-C₁-C₄-alkyl, where each cycloalkyl and each heterocyclyl ring may contain a carbonyl or thiocarbonyl ring member, and where each cycloalkyl, the phenyl and each heterocyclyl ring may be unsubstituted or may carry one, two, three or four substituents, in each case selected from the group consisting of cyano, nitro, amino, hydroxyl, carboxyl, halogen, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₁-C₄-alkylsulfonyl, C₁-C₄-haloalkylsulfonyl, (C₁-C₄-alkyl)carbonyl, (C₁-C₄-haloalkyl)carbonyl, (C₁-C₄-alkyl)carbonyloxy, (C₁-C₄-haloalkyl)carbonyloxy, (C₁-C₄-alkoxy)carbonyl and di(C₁-C₄-alkyl)amino;

R¹⁴ is hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, phenyl or phenyl-C₁-C₄-alkyl;

and the agriculturally useful salts of I.

Moreover, the invention relates to

the use of compounds I as herbicides and/or for the desiccation and/or defoliation of plants,

herbicidal compositions and compositions for the desiccation and/or defoliation of plants, which compositions comprise the compounds I as active substances,

processes for preparing the compounds I and herbicidal compositions and compositions for the desiccation and/or defoliation of plants using the compounds I, and

methods for controlling undesirable vegetation (harmful plants) and for the desiccation and/or defoliation of plants using the compounds I.

In the substituents, the compounds of the formula I may have one or more chiral centers, in which case they are present as enantiomer or diastereomer mixtures. The invention provides both the pure enantiomers or diastereomers and mixtures thereof.

Suitable agriculturally useful salts are, in particular, the salts of those cations or the acid addition salts of those acids whose cations or anions, respectively, do not negatively affect the herbicidal action of the compounds I. Thus, suitable cations are, in particular, the ions of the alkali metals, preferably sodium and potassium, of the alkaline earth metals, preferably calcium, magnesium and barium, and of the transition metals, preferably manganese, copper, zinc and iron, and the ammonium ion which, if desired, may carry one to four C₁-C₄-alkyl substituents and/or one phenyl or benzyl substituent, preferably diisopropylammonium, tetramethylammonium, tetrabutylammonium, trimethylbenzylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogen sulfate, sulfate, dihydrogen phosphate, hydrogen phosphate, phosphate, nitrate, hydrogen carbonate, carbonate, hexafluorosilicate, hexafluorophosphate, benzoate, and also the anions of C₁-C₄-alkanoic acids, preferably formate, acetate, propionate and butyrate. They can be formed by reaction of I with an acid of the corresponding anion, preferably hydrochloric acid, hydrobromic acid, sulfuric acid, phosphoric acid or nitric acid.

The organic molecule moieties mentioned in the definition of the substituents R¹, R², R⁴, R⁷ to R¹⁸ or as radicals on cycloalkyl, phenyl or heterocyclic rings or on X, Y and Z are—like the term halogen—collective terms for individual enumerations of the individual group members. All carbon chains, i.e. all alkyl, haloalkyl, phenylalkyl, cycloalkylalkyl, alkoxy, haloalkoxy, alkylthio, haloalkylthio, alkylsulfinyl, haloalkylsulfinyl, alkylsulfonyl, haloalkylsulfonyl, alkenyl, haloalkenyl, alkynyl and haloalkynyl groups and corresponding group moieties in larger groups such as alkoxycarbonyl, phenylalkyl, cycloalkylalkyl, alkoxycarbonylalkyl, etc., can be straight-chain or branched, the prefix Cₙ-Cₘ in each case denoting the possible number of carbon atoms in the group. Halogenated substituents preferably carry one, two, three, four or five identical or different halogen atoms. In each case, the term halogen denotes fluorine, chlorine, bromine or iodine.

Other examples of meanings are:

C₁-C₄-alkyl: CH₃, C₂H₅, n-propyl, CH(CH₃)₂, n-butyl, CH(CH₃)—C₂H₅, CH₂—CH(CH₃)₂ and C(CH₃)₃;

C₁-C₄-haloalkyl: a C₁-C₄-alkyl radical as mentioned above which is partially or fully substituted by fluorine, chlorine, bromine and/or iodine, i.e. for example CH₂F, CHF₂, CF₃, CH₂Cl, dichloromethyl, trichloromethyl, chlorofluoromethyl, dichlorofluoromethyl, chlorodifluoro-methyl, 2-fluoroethyl, 2-chloroethyl, 2-bromoethyl, 2-iodoethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, C₂F₅, 2-fluoropropyl, 3-fluoropropyl, 2,2-difluoropropyl, 2,3-difluoropropyl, 2-chloropropyl, 3-chloropropyl, 2,3-dichloropropyl, 2-bromopropyl, 3-bromopropyl, 3,3,3-trifluoropropyl, 3,3,3-trichloropropyl, 2,2,3,3,3-pentafluoropropyl, heptafluoropropyl, 1-fluoromethyl-2-fluoroethyl, 1-chloromethyl-2-chloroethyl, 1-bromomethyl-2-bromoethyl, 4-fluorobutyl, 4-chlorobutyl, 4-bromobutyl or nonafluorobutyl;

C₁-C₆-alkyl: C₁-C₄-alkyl as mentioned above, and also, for example, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl or 1-ethyl-2-methylpropyl, preferably methyl, ethyl, n-propyl, 1-methylethyl, n-butyl, 1,1-dimethylethyl, n-pentyl or n-hexyl;

C₁-C₆-haloalkyl: a C₁-C₆-alkyl radical as mentioned above which is partially or fully substituted by fluorine, chlorine, bromine and/or iodine, i.e. for example one of the radicals mentioned under C₁-C₄-haloalkyl, and also 5-fluoro-1-pentyl, 5-chloro-1-pentyl, 5-bromo-1-pentyl, 5-iodo-1-pentyl, 5,5,5-trichloro-1-pentyl, undecafluoro-pentyl, 6-fluoro-1-hexyl, 6-chloro-1-hexyl, 6-bromo-1-hexyl, 6-iodo-1-hexyl, 6,6,6-trichloro-1-hexyl or dodecafluorohexyl;

phenyl-C₁-C₄-alkyl: benzyl, 1-phenylethyl, 2-phenylethyl, 1-phenylprop-1-yl, 2-phenylprop-1-yl, 3-phenylprop-1-yl, 1-phenylbut-1-yl, 2-phenylbut-1-yl, 3-phenylbut-1-yl, 4-phenylbut-1-yl, 1-phenylbut-2-yl, 2-phenylbut-2-yl, 3-phenylbut-2-yl, 4-phenylbut-2-yl, 1-phenylmethyleth-1-yl, 1-phenylmethyl-1-methyleth-1-yl or 1-phenylmethylprop-1-yl, preferably benzyl or 2-phenylethyl;

heterocyclyl-C₁-C₄-alkyl: heterocyclylmethyl, 1-heterocyclylethyl, 2-heterocyclylethyl, 1-heterocyclylprop-1-yl, 2-heterocyclylprop-1-yl, 3-heterocyclylprop-1-yl, 1-heterocyclylbut-1-yl, 2-heterocyclylbut-1-yl, 3-heterocyclylbut-1-yl, 4-heterocyclylbut-1-yl, 1-heterocyclylbut-2-yl, 2-heterocyclylbut-2-yl, 3-heterocyclylbut-2-yl, 3-heterocyclylbut-2-yl, 4-heterocyclylbut-2-yl, 1-heterocyclylmethyleth-1-yl, 1-heterocyclylmethyl-1-methyleth-1-yl or 1-heterocyclylmethylprop-1-yl, preferably heterocyclylmethyl or 2-heterocyclylethyl;

C₁-C₄-alkoxy: OCH₃, OC₂H₅, n-propoxy, OCH(CH₃)₂, n-butoxy, OCH(CH₃)—C₂H₅, OCH₂—CH(CH₃)₂ or OC(CH₃)₃, preferably OCH₃, OC₂H₅ or OCH(CH₃)₂;

C₁-C₄-haloalkoxy: a C₁-C₄-alkoxy radical as mentioned above which is partially or fully substituted by fluorine, chlorine, bromine and/or iodine, i.e. for example OCH₂F, OCHF₂, OCF₃, OCH₂Cl, OCH(Cl)₂, OC(Cl)₃, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, OC₂F₅, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, 2,2,3,3,3-pentafluoropropoxy, OCF₂—C₂F₅, 1-(CH₂F)-2-fluoroethoxy, 1-(CH₂Cl)-2-chloroethoxy, 1-(CH₂Br)-2-bromoethoxy, 4-fluorobutoxy, 4-chlorobutoxy, 4-bromobutoxy or nonafluorobutoxy, preferably OCHF₂, OCF₃, dichlorofluoromethoxy, chlorodifluoromethoxy or 2,2,2-trifluoroethoxy;

C₁-C₆-alkylthio: SCH₃, SC₂H₅, n-propylthio, SCH(CH₃)₂, n-butylthio, SCH(CH₃)—C₂H₅, SCH₂—CH(CH₃)₂ or SC(CH₃)₃, preferably SCH₃ or SC₂H₅;

C₁-C₄-haloalkylthio: a C₁-C₄-alkylthio radical as mentioned above which is partially or fully substituted by fluorine, chlorine, bromine and/or iodine, i.e. for example SCH₂F, SCHF₂, SCH₂Cl, SCH(Cl)₂, SC(Cl)₃, SCF₃, chlorofluoromethylthio, dichlorofluoromethylthio, chlorodifluoromethylthio, 2-fluoroethylthio, 2-chloroethylthio, 2-bromoethylthio, 2-iodoethylthio, 2,2-difluoroethylthio, 2,2,2-trifluoroethylthio, 2-chloro-2-fluoroethylthio, 2-chloro-2,2-difluoroethylthio, 2,2-dichloro-2-fluoroethylthio, 2,2,2-trichloroethylthio, SC₂F₅, 2-fluoropropylthio, 3-fluoropropylthio, 2,2-difluoropropylthio, 2,3-difluoropropylthio, 2-chloropropylthio, 3-chloropropylthio, 2,3-dichloropropylthio, 2-bromopropylthio, 3-bromopropylthio, 3,3,3-trifluoropropylthio, 3,3,3-trichloropropylthio, SCH₂—C₂F₅, SCF₂—C₂F₅, 1-(CH₂F)-2-fluoroethylthio, 1-(CH₂Cl)-2-chloroethylthio, 1-(CH₂Br)-2-bromoethylthio, 4-fluorobutylthio, 4-chlorobutylthio, 4-bromobutylthio or SCF₂—CF₂—C₂F₅, preferably SCHF₂, SCF₃, dichlorofluoromethylthio, chlorodifluoromethylthio or 2,2,2-trifluoroethylthio;

C₁-C₄-alkoxy-C₁-C₄-alkyl: C₁-C₄-alkyl which is substituted by C₁-C₄-alkoxy as mentioned above, i.e. for example CH₂—OCH₃, CH₂—OC₂H₅, n-propoxymethyl, CH₂—OCH(CH₃)2, n-butoxymethyl, (1-methylpropoxy)methyl, (2-methylpropoxy)methyl, CH₂—OC(CH₃)₃, 2-(methoxy)ethyl, 2-(ethoxy)ethyl, 2-(n-propoxy)ethyl, 2-(1-methylethoxy)ethyl, 2-(n-butoxy)ethyl, 2-(1-methylpropoxy)ethyl, 2-(2-methylpropoxy)ethyl, 2-(1,1-dimethylethoxy)ethyl, 2-(methoxy)propyl, 2-(ethoxy)propyl, 2-(n-propoxy)propyl, 2-(1-methylethoxy)propyl, 2-(n-butoxy)propyl, 2-(1-methylpropoxy)propyl, 2-(2-methylpropoxy)propyl, 2-(1,1-dimethylethoxy)propyl, 3-(methoxy)propyl, 3-(ethoxy)propyl, 3-(n-propoxy)propyl, 3-(1-methylethoxy)propyl, 3-(n-butoxy)propyl, 3-(1-methylpropoxy)propyl, 3-(2-methylpropoxy)propyl, 3-(1,1-dimethylethoxy)propyl, 2-(methoxy)butyl, 2-(ethoxy)butyl, 2-(n-propoxy)butyl, 2-(1-methylethoxy)butyl, 2-(n-butoxy)butyl, 2-(1-methylpropoxy)butyl, 2-(2-methylpropoxy)butyl, 2-(1,1-dimethylethoxy)butyl, 3-(methoxy)butyl, 3-(ethoxy)butyl, 3-(n-propoxy)butyl, 3-(1-methylethoxy)butyl, 3-(n-butoxy)butyl, 3-(1-methylpropoxy)butyl, 3-(2-methylpropoxy)butyl, 3-(1,1-dimethylethoxy)butyl, 4-(methoxy)butyl, 4-(ethoxy)butyl, 4-(n-propoxy)butyl, 4-(1-methylethoxy)butyl, 4-(n-butoxy)butyl, 4-(1-methylpropoxy)butyl, 4-(2-methylpropoxy)butyl or 4-(1,1-dimethylethoxy)butyl, preferably CH₂—OCH₃, CH₂—OC₂H₅, 2-methoxyethyl or 2-ethoxyethyl;

C₁-C₄-alkylthio-C₁-C₄-alkyl: C₁-C₄-alkyl which is substituted by C₁-C₄-alkylthio as mentioned above, i.e. for example CH₂—SCH₃, CH₂—SC₂H₅, n-propylthiomethyl, CH₂—SCH(CH₃)₂, n-butylthiomethyl, (1-methylpropylthio)methyl, (2-methylpropylthio)methyl, CH₂—SC(CH₃)₂, 2-(methylthio)ethyl, 2-(ethylthio)ethyl, 2-(n-propylthio)ethyl, 2-(1-methylethylthio)ethyl, 2-(n-butylthio)ethyl, 2-(1-methylpropylthio)ethyl, 2-(2-methylpropylthio)ethyl, 2-(1,1-dimethylethylthio)ethyl, 2-(methylthio)propyl, 2-(ethylthio)propyl, 2-(n-propylthio)propyl, 2-(1-methylethylthio)propyl, 2-(n-butylthio)propyl, 2-(1-methylpropylthio)propyl, 2-(2-methylpropylthio)propyl, 2-(1,1-dimethylethylthio)propyl, 3-(methylthio)propyl, 3-(ethylthio)propyl, 3-(n-propylthio)propyl, 3-(1-methylethylthio)propyl, 3-(n-butylthio)propyl, 3-(1-methylpropylthio)propyl, 3-(2-methylpropylthio)propyl, 3-(1,1-dimethylethylthio)propyl, 2-(methylthio)butyl, 2-(ethylthio)butyl, 2-(n-propylthio)butyl, 2-(1-methylethylthio)butyl, 2-(n-butylthio)butyl, 2-(1-methylpropylthio)butyl, 2-(2-methylpropylthio)butyl, 2-(1,1-dimethylethylthio)butyl, 3-(methylthio)butyl, 3-(ethylthio)butyl, 3-(n-propylthio)butyl, 3-(1-methylethylthio)butyl, 3-(n-butylthio)butyl, 3-(1-methylpropylthio)butyl, 3-(2-methylpropylthio)butyl, 3-(1,1-dimethylethylthio)butyl, 4-(methylthio)butyl, 4-(ethylthio)butyl, 4-(n-propylthio)butyl, 4-(1-methylethylthio)butyl, 4-(n-butylthio)butyl, 4-(1-methylpropylthio)butyl, 4-(2-methylpropylthio)butyl or 4-(1,1-dimethylethylthio)butyl, preferably CH₂—SCH₃, CH₂—SC₂H₅, 2-methylthioethyl or 2-ethylthioethyl;

(C₁-C₄-alkyl)carbonyl: CO—CH₃, CO—C₂H₅, CO—CH₂—C₂H₅, CO—CH(CH₃)₂, n-butylcarbonyl, CO—CH(CH₃)—C₂H₅, CO—CH₂—CH(CH₃)₂ or CO—C(CH₃)₃, preferably CO—CH₃ or CO—C₂H₅;

(C₁-C₄-haloalkyl)carbonyl: a (C₁-C₄-alkyl)carbonyl radical as mentioned above which is partially or fully substituted by fluorine, chlorine, bromine and/or iodine, i.e. for example CO—CH₂F, CO—CHF₂, CO—CF₃, CO—CH₂Cl, CO—CH(Cl)₂, CO—C(Cl)₃, chlorofluoromethylcarbonyl, dichlorofluoromethylcarbonyl, chlorodifluoromethylcarbonyl, 2-fluoroethylcarbonyl, 2-chloroethylcarbonyl, 2-bromoethylcarbonyl, 2-iodoethylcarbonyl, 2,2-difluoroethylcarbonyl, 2,2,2-trifluoroethylcarbonyl, 2-chloro-2-fluoroethylcarbonyl, 2-chloro-2,2-difluoroethylcarbonyl, 2,2-dichloro-2-fluoroethylcarbonyl, 2,2,2-trichloroethylcarbonyl, CO—C₂F₅, 2-fluoropropylcarbonyl, 3-fluoropropylcarbonyl, 2,2-difluoropropylcarbonyl, 2,3-difluoropropylcarbonyl, 2-chloropropylcarbonyl, 3-chloropropylcarbonyl, 2,3-dichloropropylcarbonyl, 2-bromopropylcarbonyl, 3-bromopropylcarbonyl, 3,3,3-trifluoropropylcarbonyl, 3,3,3-trichloropropylcarbonyl, 2,2,3,3,3-pentafluoropropylcarbonyl, CO—CF₂—C₂F₅, 1-(CH₂F)-2-fluoroethylcarbonyl, 1-(CH₂Cl)-2-chloroethylcarbonyl, 1-(CH₂Br)-2-bromoethylcarbonyl, 4-fluorobutylcarbonyl, 4-chlorobutylcarbonyl, 4-bromobutylcarbonyl or nonafluorobutylcarbonyl, preferably CO—CF₃, CO—CH₂Cl or 2,2,2-trifluoroethylcarbonyl;

(C₁-C₄-alkyl)carbonyloxy: O—CO—CH₃, O—CO—C₂H₅, O—CO—CH₂—C₂H₅, O—CO—CH(CH₃)₂, O—CO—CH₂—CH₂—C₂H₅, O—CO—CH(CH₃)—C₂H₅, O—CO—CH₂—CH(CH₃)₂ or O—CO—C(CH₃)₃, preferably O—CO—CH₃ or O—CO—C₂H₅;

(C₁-C₄-haloalkyl)carbonyloxy: a (C₁-C₄-alkyl)carbonyl radical as mentioned above which is partially or fully substituted by fluorine, chlorine, bromine and/or iodine, i.e. for example O—CO—CH₂F, O—CO—CHF₂, O—CO—CF₃, O—CO—CH₂Cl, O—CO—CH(Cl)₂, O—CO—C(Cl)₃, chlorofluoromethylcarbonyloxy, dichlorofluoromethylcarbonyloxy, chlorodifluoromethylcarbonyloxy, 2-fluoroethylcarbonyloxy, 2-chloroethylcarbonyloxy, 2-bromoethylcarbonyloxy, 2-iodoethylcarbonyloxy, 2,2-difluoroethylcarbonyloxy, 2,2,2-trifluoroethylcarbonyloxy, 2-chloro-2-fluoroethylcarbonyloxy, 2-chloro-2,2-difluoroethylcarbonyloxy, 2,2-dichloro-2-fluoroethylcarbonyloxy, 2,2,2-trichloroethylcarbonyloxy, O—CO—C₂F₅, 2-fluoropropylcarbonyloxy, 3-fluoropropylcarbonyloxy, 2,2-difluoropropylcarbonyloxy, 2,3-difluoropropylcarbonyloxy, 2-chloropropylcarbonyloxy, 3-chloropropylcarbonyloxy, 2,3-dichloropropylcarbonyloxy, 2-bromopropylcarbonyloxy, 3-bromopropylcarbonyloxy, 3,3,3-trifluoropropylcarbonyloxy, 3,3,3-trichloropropylcarbonyloxy, 2,2,3,3,3-pentafluoropropylcarbonyloxy, heptafluoropropylcarbonyloxy, 1-(CH₂F)-2-fluoroethylcarbonyloxy, 1-(CH₂Cl)-2-chloroethylcarbonyloxy, 1-(CH₂Br)-2-bromoethylcarbonyloxy, 4-fluorobutylcarbonyloxy, 4-chlorobutylcarbonyloxy, 4-bromobutylcarbonyloxy or nonafluorobutylcarbonyloxy, preferably O—CO—CF₃, O—CO—CH₂Cl or 2,2,2-trifluoroethylcarbonyloxy;

(C₁-C₄-alkoxy)carbonyl: CO—OCH₃, CO—OC₂H₅, n-propoxycarbonyl, CO—OCH(CH₃)₂, n-butoxycarbonyl, CO—OCH(CH₃)—C₂H₅, CO—OCH₂—CH(CH₃)₂ or CO—OC(CH₃)₃, preferably CO—OCH₃ or CO—OC₂H₅;

(C₁-C₄-alkoxy)carbonyl-C₁-C₄-alkyl: C₁-C₄-alkyl which is substituted by (C₁-C₄-alkoxy)carbonyl as mentioned above, i.e. for example methoxycarbonylmethyl, ethoxycarbonylmethyl, n-propoxycarbonylmethyl, (1-methylethoxycarbonyl)methyl, n-butoxycarbonylmethyl, (1-methylpropoxycarbonyl)methyl, (2-methylpropoxycarbonyl)methyl, (1,1-dimethylethoxycarbonyl)methyl, 1-(methoxycarbonyl)ethyl, 1-(ethoxycarbonyl)ethyl, 1-(n-propoxycarbonyl)ethyl, 1-(1-methylethoxycarbonyl)ethyl, 1-(n-butoxycarbonyl)ethyl, 2-(methoxycarbonyl)ethyl, 2-(ethoxycarbonyl)ethyl, 2-(n-propoxycarbonyl)ethyl, 2-(1-methylethoxycarbonyl)ethyl, 2-(n-butoxycarbonyl)ethyl, 2-(1-methylpropoxycarbonyl)ethyl, 2-(2-methylpropoxycarbonyl)ethyl, 2-(1,1-dimethylethoxycarbonyl)ethyl, 2-(methoxycarbonyl)propyl, 2-(ethoxycarbonyl)propyl, 2-(n-propoxycarbonyl)propyl, 2-(1-methylethoxycarbonyl)propyl, 2-(n-butoxycarbonyl)propyl, 2-(1-methylpropoxycarbonyl)propyl, 2-(2-methylpropoxycarbonyl)propyl, 2-(1,1-dimethylethoxycarbonyl)propyl, 3-(methoxycarbonyl)propyl, 3-(ethoxycarbonyl)propyl, 3-(n-propoxycarbonyl)propyl, 3-(1-methylethoxycarbonyl)propyl, 3-(n-butoxycarbonyl)propyl, 3-(1-methylpropoxycarbonyl)propyl, 3-(2-methylpropoxycarbonyl)propyl, 3-(1,1-dimethylethoxycarbonyl)propyl, 2-(methoxycarbonyl)butyl, 2-(ethoxycarbonyl)butyl, 2-(n-propoxycarbonyl)butyl, 2-(1-methylethoxycarbonyl)butyl, 2-(n-butoxycarbonyl)butyl, 2-(1-methylpropoxycarbonyl)butyl, 2-(2-methylpropoxycarbonyl)butyl, 2-(1,1-dimethylethoxycarbonyl)butyl, 3-(methoxycarbonyl)butyl, 3-(ethoxycarbonyl)butyl, 3-(n-propoxycarbonyl)butyl, 3-(1-methylethoxycarbonyl)butyl, 3-(n-butoxycarbonyl)butyl, 3-(1-methylpropoxycarbonyl)butyl, 3-(2-methylpropoxycarbonyl)butyl, 3-(1,1-dimethylethoxycarbonyl)butyl, 4-(methoxycarbonyl)butyl, 4-(ethoxycarbonyl)butyl, 4-(n-propoxycarbonyl)butyl, 4-(1-methylethoxycarbonyl)butyl, 4-(n-butoxycarbonyl)butyl, 4-(1-methylpropoxycarbonyl)butyl, 4-(2-methylpropoxycarbonyl)butyl or 4-(1,1-dimethylethoxycarbonyl)butyl, preferably methoxycarbonylmethyl, ethoxycarbonylmethyl, 1-(methoxycarbonyl)ethyl or 1-(ethoxycarbonyl)ethyl;

(C₁-C₄-alkoxy)carbonyl-C₁-C₄-alkoxy: C₁-C₄-alkoxy which is substituted by (C₁-C₄-alkoxy)carbonyl as mentioned above, i.e. for example methoxycarbonylmethoxy, ethoxycarbonylmethoxy, n-propoxycarbonylmethoxy, (1-methylethoxycarbonyl)methoxy, n-butoxycarbonylmethoxy, (1-methylpropoxycarbonyl)methoxy, (2-methylpropoxycarbonyl)methoxy, (1,1-dimethylethoxycarbonyl)methoxy, 1-(methoxycarbonyl)ethoxy, 1-(ethoxycarbonyl)ethoxy, 1-(n-propoxycarbonyl)ethoxy, 1-(1-methylethoxycarbonyl)ethoxy, 1-(n-butoxycarbonyl)ethoxy, 2-(methoxycarbonyl)ethoxy, 2-(ethoxycarbonyl)ethoxy, 2-(n-propoxycarbonyl)ethoxy, 2-(1-methylethoxycarbonyl)ethoxy, 2-(n-butoxycarbonyl)ethoxy, 2-(1-methylpropoxycarbonyl)ethoxy, 2-(2-methylpropoxycarbonyl)ethoxy, 2-(1,1-dimethylethoxycarbonyl)ethoxy, 2-(methoxycarbonyl)propoxy, 2-(ethoxycarbonyl)propoxy, 2-(n-propoxycarbonyl)propoxy, 2-(1-methylethoxycarbonyl)propoxy, 2-(n-butoxycarbonyl)propoxy, 2-(1-methylpropoxycarbonyl)propoxy, 2-(2-methylpropoxycarbonyl)propoxy, 2-(1,1-dimethylethoxycarbonyl)propoxy, 3-(methoxycarbonyl)propoxy, 3-(ethoxycarbonyl)propoxy, 3-(n-propoxycarbonyl)propoxy, 3-(1-methylethoxycarbonyl)propoxy, 3-(n-butoxycarbonyl)propoxy, 3-(1-methylpropoxycarbonyl)propoxy, 3-(2-methylpropoxycarbonyl)propoxy, 3-(1,1-dimethylethoxycarbonyl)propoxy, 2-(methoxycarbonyl)butoxy, 2-(ethoxycarbonyl)butoxy, 2-(n-propoxycarbonyl)butoxy, 2-(1-methylethoxycarbonyl)butoxy, 2-(n-butoxycarbonyl)butoxy, 2-(1-methylpropoxycarbonyl)butoxy, 2-(2-methylpropoxycarbonyl)butoxy, 2-(1,1-dimethylethoxycarbonyl)butoxy, 3-(methoxycarbonyl)butoxy, 3-(ethoxycarbonyl)butoxy, 3-(n-propoxycarbonyl)butoxy, 3-(1-methylethoxycarbonyl)butoxy, 3-(n-butoxycarbonyl)butoxy, 3-(1-methylpropoxycarbonyl)butoxy, 3-(2-methylpropoxycarbonyl)butoxy, 3-(1,1-dimethylethoxycarbonyl)butoxy, 4-(methoxycarbonyl)butoxy, 4-(ethoxycarbonyl)butoxy, 4-(n-propoxycarbonyl)butoxy, 4-(1-methylethoxycarbonyl)butoxy, 4-(n-butoxycarbonyl)butoxy, 4-(1-methylpropoxycarbonyl)butoxy, 4-(2-methylpropoxycarbonyl)butyl or 4-(1,1-dimethylethoxycarbonyl)butoxy, preferably methoxycarbonylmethoxy, ethoxycarbonylmethoxy, 1-(methoxycarbonyl)ethoxy or 1-(ethoxycarbonyl)ethoxy;

(C₁-C₄-alkoxy)carbonyl-C₁-C₄-alkylthio: C₁-C₄-alkylthio which is substituted by (C₁-C₄-alkoxy)carbonyl as mentioned above, i.e. for example methoxycarbonylmethylthio, ethoxycarbonylmethylthio, n-propoxycarbonylmethylthio, (1-methylethoxycarbonyl)methylthio, n-butoxycarbonylmethylthio, (1-methylpropoxycarbonyl)methylthio, (2-methylpropoxycarbonyl)methylthio, (1,1-dimethylethoxycarbonyl)methylthio, 1-(methoxycarbonyl)ethylthio, 1-(ethoxycarbonyl)ethylthio, 1-(n-propoxycarbonyl)ethylthio, 1-(1-methylethoxycarbonyl)ethylthio, 1-(n-butoxycarbonyl)ethylthio, 2-(methoxycarbonyl)ethylthio, 2-(ethoxycarbonyl)ethylthio, 2-(n-propoxycarbonyl)ethylthio, 2-(1-methylethoxycarbonyl)ethylthio, 2-(n-butoxycarbonyl)ethylthio, 2-(1-methylpropoxycarbonyl)ethylthio, 2-(2-methylpropoxycarbonyl)ethylthio, 2-(1,1-dimethylethoxycarbonyl)ethylthio, 2-(methoxycarbonyl)propylthio, 2-(ethoxycarbonyl)propylthio, 2-(n-propoxycarbonyl)propylthio, 2-(1-methylethoxycarbonyl)propylthio, 2-(n-butoxycarbonyl)propylthio, 2-(1-methylpropoxycarbonyl)propylthio, 2-(2-methylpropoxycarbonyl)propylthio, 2-(1,1-dimethylethoxycarbonyl)propylthio, 3-(methoxycarbonyl)propylthio, 3-(ethoxycarbonyl)propylthio, 3-(n-propoxycarbonyl)propylthio, 3-(1-methylethoxycarbonyl)propylthio, 3-(n-butoxycarbonyl)propylthio, 3-(1-methylpropoxycarbonyl)propylthio, 3-(2-methylpropoxycarbonyl)propylthio, 3-(1,1-dimethylethoxycarbonyl)propylthio, 2-(methoxycarbonyl)butylthio, 2-(ethoxycarbonyl)butylthio, 2-(n-propoxycarbonyl)butylthio, 2-(1-methylethoxycarbonyl)butylthio, 2-(n-butoxycarbonyl)butylthio, 2-(1-methylpropoxycarbonyl)butylthio, 2-(2-methylpropoxycarbonyl)butylthio, 2-(1,1-dimethylethoxycarbonyl)butylthio, 3-(methoxycarbonyl)butylthio, 3-(ethoxycarbonyl)butylthio, 3-(n-propoxycarbonyl)butylthio, 3-(1-methylethoxycarbonyl)butylthio, 3-(n-butoxycarbonyl)butylthio, 3-(1-methylpropoxycarbonyl)butylthio, 3-(2-methylpropoxycarbonyl)butylthio, 3-(1,1-dimethylethoxycarbonyl)butylthio, 4-(methoxycarbonyl)butylthio, 4-(ethoxycarbonyl)butylthio, 4-(n-propoxycarbonyl)butylthio, 4-(1-methylethoxycarbonyl)butylthio, 4-(n-butoxycarbonyl)butylthio, 4-(1-methylpropoxycarbonyl)butylthio, 4-(2-methylpropoxycarbonyl)butyl or 4-(1,1-dimethylethoxycarbonyl)butylthio, preferably methoxycarbonylmethylthio, ethoxycarbonylmethylthio, 1-(methoxycarbonyl)ethylthio or 1-(ethoxycarbonyl)ethylthio;

C₁-C₄-alkylsulfinyl: SO—CH₃, SO—C₂H₅, SO—CH₂—C₂H₅, SO—CH(CH₃)₂, n-butylsulfinyl, SO—CH(CH₃)—C₂H₅, SO—CH₂—CH(CH₃)₂ or SO—C(CH₃)₃, preferably SO—CH₃ or SO—C₂H₅;

C₁-C₄-haloalkylsulfinyl: a C₁-C₄-alkylsulfinyl radical as mentioned above which is partially or fully substituted by fluorine, chlorine, bromine and/or iodine, i.e. for example SO—CH₂F, SO—CHF₂, SO—CF₃, SO—CH₂Cl, SO—CH(Cl)₂, SO—C(Cl)₃, chlorofluoromethylsulfinyl, dichlorofluoromethylsulfinyl, chlorodifluoromethylsulfinyl, 2-fluoroethylsulfinyl, 2-chloroethylsulfinyl, 2-bromoethylsulfinyl, 2-iodoethylsulfinyl, 2,2-difluoroethylsulfinyl, 2,2,2-trifluoroethylsulfinyl, 2-chloro-2-fluoroethylsulfinyl, 2-chloro-2,2-difluoroethylsulfinyl, 2,2-dichloro-2-fluoroethylsulfinyl, 2,2,2-trichloroethylsulfinyl, SO—C₂F₅, 2-fluoropropylsulfinyl, 3-fluoropropylsulfinyl, 2,2-difluoropropylsulfinyl, 2,3-difluoropropylsulfinyl, 2-chloropropylsulfinyl, 3-chloropropylsulfinyl, 2,3-dichloropropylsulfinyl, 2-bromopropylsulfinyl, 3-bromopropylsulfinyl, 3,3,3-trifluoropropylsulfinyl, 3,3,3-trichloropropylsulfinyl, SO—CH₂—C₂F₅, SO—CF₂—C₂F₅, 1-(fluoromethyl)-2-fluoroethylsulfinyl, 1-(chloromethyl)-2-chloroethylsulfinyl, 1-(bromomethyl)-2-bromoethylsulfinyl, 4-fluorobutylsulfinyl, 4-chlorobutylsulfinyl, 4-bromobutylsulfinyl or nonafluorobutylsulfinyl, preferably SO—CF₃, SO—CH₂Cl or 2,2,2-trifluoroethylsulfinyl;

C₁-C₄-alkylsulfonyl: SO₂—CH₃, SO₂—C₂H₅, SO₂—CH₂—C₂H₅, SO₂—CH(CH₃)₂, n-butylsulfonyl, SO₂—CH(CH₃)—C₂H₅, SO₂—CH₂—CH(CH₃)₂ or SO₂—C(CH₃)₃, preferably SO₂—CH₃ or SO₂—C₂H₅;

C₁-C₄-haloalkylsulfonyl: a C₁-C₄-alkylsulfonyl radical as mentioned above which is partially or fully substituted by fluorine, chlorine, bromine and/or iodine, i.e. for example SO₂—CH₂F, SO₂—CHF₂, SO₂—CF₃, SO₂—CH₂Cl, SO₂—CH(Cl)₂, SO₂—C(Cl)₃, chlorofluoromethylsulfonyl, dichlorofluoromethylsulfonyl, chlorodifluoromethylsulfonyl, 2-fluoroethylsulfonyl, 2-chloroethylsulfonyl, 2-bromoethylsulfonyl, 2-iodoethylsulfonyl, 2,2-difluoroethylsulfonyl, 2,2,2-trifluoroethylsulfonyl, 2-chloro-2-fluoroethylsulfonyl, 2-chloro-2,2-difluoroethylsulfonyl, 2,2-dichloro-2-fluoroethylsulfonyl, 2,2,2-trichloroethylsulfonyl, SO₂—C₂F₅, 2-fluoropropylsulfonyl, 3-fluoropropylsulfonyl, 2,2-difluoropropylsulfonyl, 2,3-difluoropropylsulfonyl, 2-chloropropylsulfonyl, 3-chloropropylsulfonyl, 2,3-dichloropropylsulfonyl, 2-bromopropylsulfonyl, 3-bromopropylsulfonyl, 3,3,3-trifluoropropylsulfonyl, 3,3,3-trichloropropylsulfonyl, SO₂—CH₂—C₂F₅, SO₂—CF₂—C₂F₅, 1-(fluoromethyl)-2-fluoroethylsulfonyl, 1-(chloromethyl)-2-chloroethylsulfonyl, 1-(bromomethyl)-2-bromoethylsulfonyl, 4-fluorobutylsulfonyl, 4-chlorobutylsulfonyl, 4-bromobutylsulfonyl or nonafluorobutylsulfonyl, preferably SO₂—CF₃, SO₂—CH₂Cl or 2,2,2-trifluoroethylsulfonyl;

di(C₁-C₄-alkyl)amino: N(CH₃)₂, N(C₂H₅)₂, N,N-dipropylamino, N[CH(CH₃)₂]₂, N,N-dibutylamino, N,N-di(1-methylpropyl)amino, N,N-di(2-methylpropyl)amino, N[C(CH₃)₃]2, N-ethyl-N-methylamino, N-methyl-N-propylamino, N-methyl-N-(1-methylethyl)amino, N-butyl-N-methylamino, N-methyl-N-(1-methylpropyl)amino, N-methyl-N-(2-methylpropyl)amino, N-(1,1-dimethylethyl)-N-methylamino, N-ethyl-N-propylamino, N-ethyl-N-(1-methylethyl)amino, N-butyl-N-ethylamino, N-ethyl-N-(1-methylpropyl)amino, N-ethyl-N-(2-methylpropyl)amino, N-ethyl-N-(1,1-dimethylethyl)amino, N-(1-methylethyl)-N-propylamino, N-butyl-N-propylamino, N-(1-methylpropyl)-N-propylamino, N-(2-methylpropyl)-N-propylamino, N-(1,1-dimethylethyl)-N-propylamino, N-butyl-N-(1-methylethyl)amino, N-(1-methylethyl)-N-(1-methylpropyl)amino, N-(1-methylethyl)-N-(2-methylpropyl)amino, N-(1,1-dimethylethyl)-N-(1-methylethyl)amino, N-butyl-N-(1-methylpropyl)amino, N-butyl-N-(2-methylpropyl)amino, N-butyl-N-(1,1-dimethylethyl)amino, N-(1-methylpropyl)-N-(2-methylpropyl)amino, N-(1,1-dimethylethyl)-N-(1-methylpropyl)amino or N-(1,1-dimethylethyl)-N-(2-methylpropyl)amino, preferably N(CH₃)₂ or N(C₂H₅);

di(C₁-C₄-alkyl)aminocarbonyl: for example N,N-dimethylaminocarbonyl, N,N-diethylaminocarbonyl, N,N-di(1-methylethyl)aminocarbonyl, N,N-dipropylaminocarbonyl, N,N-dibutylaminocarbonyl, N,N-di(1-methylpropyl)aminocarbonyl, N,N-di(2-methylpropyl)aminocarbonyl, N,N-di(1,1-dimethylethyl)aminocarbonyl, N-ethyl-N-methylaminocarbonyl, N-methyl-N-propylaminocarbonyl, N-methyl-N-(1-methylethyl)aminocarbonyl, N-butyl-N-methylaminocarbonyl, N-methyl-N-(1-methylpropyl)aminocarbonyl, N-methyl-N-(2-methylpropyl)aminocarbonyl, N-(1,1-dimethylethyl)-N-methylaminocarbonyl, N-ethyl-N-propylaminocarbonyl, N-ethyl-N-(1-methylethyl)aminocarbonyl, N-butyl-N-ethylaminocarbonyl, N-ethyl-N-(1-methylpropyl)aminocarbonyl, N-ethyl-N-(2-methylpropyl)aminocarbonyl, N-ethyl-N-(1,1-dimethylethyl)aminocarbonyl, N-(1-methylethyl)-N-propylaminocarbonyl, N-butyl-N-propylaminocarbonyl, N-(1-methylpropyl)-N-propylaminocarbonyl, N-(2-methylpropyl)-N-propylaminocarbonyl, N-(1,1-dimethylethyl)-N-propylaminocarbonyl, N-butyl-N-(1-methylethyl)aminocarbonyl, N-(1-methylethyl)-N-(1-methylpropyl)aminocarbonyl, N-(1-methylethyl)-N-(2-methylpropyl)aminocarbonyl, N-(1,1-dimethylethyl)-N-(1-methylethyl)aminocarbonyl, N-butyl-N-(1-methylpropyl)aminocarbonyl, N-butyl-N-(2-methylpropyl)aminocarbonyl, N-butyl-N-(1,1-dimethylethyl)aminocarbonyl, N-(1-methylpropyl)-N-(2-methylpropyl)aminocarbonyl, N-(1,1-dimethylethyl)-N-(1-methylpropyl)aminocarbonyl or N-(1,1-dimethylethyl)-N-(2-methylpropyl)aminocarbonyl;

di(C₁-C₄-alkyl)aminocarbonyl-C₁-C₄-alkyl: C₁-C₄-alkyl which is monosubstituted by di(C₁-C₄-alkyl)aminocarbonyl, for example di(C₁-C₄-alkyl)aminocarbonylmethyl, 1- or 2-di(C₁-C₄-alkyl)aminocarbonylethyl, 1-, 2- or 3-di(C₁-C₄-alkyl)aminocarbonylpropyl;

di(C₁-C₄-alkyl)aminocarbonyl-C₁-C₄-alkoxy: C₁-C₄-alkoxy which is monosubstituted by di(C₁-C₄-alkyl)aminocarbonyl, for example di(C₁-C₄-alkyl)aminocarbonylmethoxy, 1- or 2-di(C₁-C₄-alkyl)aminocarbonylethoxy, 1-, 2- or 3-di(C₁-C₄-alkyl)aminocarbonylpropoxy;

di(C₁-C₄-alkyl)aminocarbonyl-C₁-C₄-alkyl: C₁-C₄-alkylthio which is monosubstituted by di(C₁-C₄-alkyl)aminocarbonyl, for example di(C₁-C₄-alkyl)aminocarbonylmethylthio, 1- or 2-di(C₁-C₄-alkyl)aminocarbonylethylthio, 1-, 2- or 3-di(C₁-C₄-alkyl)aminocarbonylpropylthio;

C₂-C₆-alkenyl: vinyl, prop-1-en-1-yl, allyl, 1-methylethenyl, 1-buten-1-yl, 1-buten-2-yl, 1-buten-3-yl, 2-buten-1-yl, 1-methylprop-1-en-1-yl, 2-methylprop-1-en-1-yl, 1-methylprop-2-en-1-yl, 2-methylprop-2-en-1-yl, n-penten-1-yl, n-penten-2-yl, n-penten-3-yl, n-penten-4-yl, 1-methylbut-1-en-1-yl, 2-methylbut-1-en-1-yl, 3-methylbut-1-en-1-yl, 1-methylbut-2-en-1-yl, 2-methylbut-2-en-1-yl, 3-methylbut-2-en-1-yl, 1-methylbut-3-en-1-yl, 2-methylbut-3-en-1-yl, 3-methylbut-3-en-1-yl, 1,1-dimethylprop-2-en-1-yl, 1,2-dimethylprop-1-en-1-yl, 1,2-dimethylprop-2-en-1-yl, 1-ethylprop-1-en-2-yl, 1-ethylprop-2-en-1-yl, n-hex-1-en-1-yl, n-hex-2-en-1-yl, n-hex-3-en-1-yl, n-hex-4-en-1-yl, n-hex-5-en-1-yl, 1-methylpent-1-en-1-yl, 2-methylpent-1-en-1-yl, 3-methylpent-1-en-1-yl, 4-methylpent-1-en-1-yl, 1-methylpent-2-en-1-yl, 2-methylpent-2-en-1-yl, 3-methylpent-2-en-1-yl, 4-methylpent-2-en-1-yl, 1-methylpent-3-en-1-yl, 2-methylpent-3-en-1-yl, 3-methylpent-3-en-1-yl, 4-methylpent-3-en-1-yl, 1-methylpent-4-en-1-yl, 2-methylpent-4-en-1-yl, 3-methylpent-4-en-1-yl, 4-methylpent-4-en-1-yl, 1,1-dimethylbut-2-en-1-yl, 1,1-dimethylbut-3-en-1-yl, 1,2-dimethylbut-1-en-1-yl, 1,2-dimethylbut-2-en-1-yl, 1,2-dimethylbut-3-en-1-yl, 1,3-dimethylbut-1-en-1-yl, 1,3-dimethylbut-2-en-1-yl, 1,3-dimethylbut-3-en-1-yl, 2,2-dimethylbut-3-en-1-yl, 2,3-dimethylbut-1-en-1-yl, 2,3-dimethylbut-2-en-1-yl, 2,3-dimethylbut-3-en-1-yl, 3,3-dimethylbut-1-en-1-yl, 3,3-dimethylbut-2-en-1-yl, 1-ethylbut-1-en-1-yl, 1-ethylbut-2-en-1-yl, 1-ethylbut-3-en-1-yl, 2-ethylbut-1-en-1-yl, 2-ethylbut-2-en-1-yl, 2-ethylbut-3-en-1-yl, 1,1,2-trimethylprop-2-en-1-yl, 1-ethyl-1-methylprop-2-en-1-yl, 1-ethyl-2-methylprop-1-en-1-yl or 1-ethyl-2-methylprop-2-en-1-yl;

C₂-C₆-haloalkenyl: C₂-C₆-alkenyl as mentioned above which is partially or fully substituted by fluorine, chlorine and/or bromine, i.e. for example 2-chlorovinyl, 2-chloroallyl, 3-chloroallyl, 2,3-dichloroallyl, 3,3-dichloroallyl, 2,3,3-trichloroallyl, 2,3-dichlorobut-2-enyl, 2-bromoallyl, 3-bromoallyl, 2,3-dibromoallyl, 3,3-dibromoallyl, 2,3,3-tribromoallyl and 2,3-dibromobut-2-enyl, preferably C₃- or C₄-haloalkenyl;

C₂-C₆-alkynyl: ethynyl and C₃-C₆-alkynyl, such as prop-1-yn-1-yl, prop-2-yn-1-yl, n-but-1-yn-1-yl, n-but-1-yn-3-yl, n-but-1-yn-4-yl, n-but-2-yn-1-yl, n-pent-1-yn-1-yl, n-pent-1-yn-3-yl, n-pent-1-yn-4-yl, n-pent-1-yn-5-yl, n-pent-2-yn-1-yl, n-pent-2-yn-4-yl, n-pent-2-yn-5-yl, 3-methylbut-1-yn-3-yl, 3-methylbut-1-yn-4-yl, n-hex-1-yn-1-yl, n-hex-1-yn-3-yl, n-hex-1-yn-4-yl, n-hex-1-yn-5-yl, n-hex-1-yn-6-yl, n-hex-2-yn-1-yl, n-hex-2-yn-4-yl, n-hex-2-yn-5-yl, n-hex-2-yn-6-yl, n-hex-3-yn-1-yl, n-hex-3-yn-2-yl, 3-methylpent-1-yn-1-yl, 3-methylpent-1-yn-3-yl, 3-methylpent-1-yn-4-yl, 3-methylpent-1-yn-5-yl, 4-methylpent-1-yn-1-yl, 4-methylpent-2-yn-4-yl or 4-methylpent-2-yn-5-yl, preferably prop-2-yn-1-yl;

C₂-C₆-haloalkynyl: C₂-C₆-alkynyl as mentioned above which is partially or fully substituted by fluorine, chlorine and/or bromine, i.e. for example 1,1-difluoroprop-2-yn-1-yl, 1,1-difluorobut-2-yn-1-yl, 4-fluorobut-2-yn-1-yl, 4-chlorobut-2-yn-1-yl, 5-fluoropent-3-yn-1-yl or 6-fluorohex-4-yn-1-yl, preferably C₃- or C₄-haloalkynyl;

C₃-C₈-cycloalkyl: cyclopropyl, cyclobutyl, cyclopentyl, cyclohexyl, cycloheptyl or cyclooctyl;

C₃-C₈-cycloalkyl containing a carbonyl or thiocarbonyl ring member: for example cyclobutanon-2-yl, cyclobutanon-3-yl, cyclopentanon-2-yl, cyclopentanon-3-yl, cyclohexanon-2-yl, cyclohexanon-4-yl, cycloheptanon-2-yl, cyclooctanon-2-yl, cyclobutanethion-2-yl, cyclobutanethion-3-yl, cyclopentanethion-2-yl, cyclopentanethion-3-yl, cyclohexanethion-2-yl, cyclohexanethion-4-yl, cycloheptanethion-2-yl or cyclooctanethion-2-yl, preferably cyclopentanon-2-yl or cyclohexanon-2-yl;

C₃-C₈-cycloalkyl-C₁-C₄-alkyl: cyclopropylmethyl, 1-cyclopropylethyl, 2-cyclopropylethyl, 1-cyclopropylprop-1-yl, 2-cyclopropylprop-1-yl, 3-cyclopropylprop-1-yl, 1-cyclopropylbut-1-yl, 2-cyclopropylbut-1-yl, 3-cyclopropylbut-1-yl, 4-cyclopropylbut-1-yl, 1-cyclopropylbut-2-yl, 2-cyclopropylbut-2-yl, 3-cyclopropylbut-2-yl, 3-cyclopropylbut-2-yl, 4-cyclopropylbut-2-yl, 1-(cyclopropylmethyl)eth-1-yl, 1-(cyclopropylmethyl)-1-(methyl)eth-1-yl, 1-(cyclopropylmethyl)prop-1-yl, cyclobutylmethyl, 1-cyclobutylethyl, 2-cyclobutylethyl, 1-cyclobutylprop-1-yl, 2-cyclobutylprop-1-yl, 3-cyclobutylprop-1-yl, 1-cyclobutylbut-1-yl, 2-cyclobutylbut-1-yl, 3-cyclobutylbut-1-yl, 4-cyclobutylbut-1-yl, 1-cyclobutylbut-2-yl, 2-cyclobutylbut-2-yl, 3-cyclobutylbut-2-yl, 3-cyclobutylbut-2-yl, 4-cyclobutylbut-2-yl, 1-(cyclobutylmethyl)eth-1-yl, 1-(cyclobutylmethyl)-1-(methyl)eth-1-yl, 1-(cyclobutylmethyl)prop-1-yl, cyclopentylmethyl, 1-cyclopentylethyl, 2-cyclopentylethyl, 1-cyclopentylprop-1-yl, 2-cyclopentylprop-1-yl, 3-cyclopentylprop-1-yl, 1-cyclopentylbut-1-yl, 2-cyclopentylbut-1-yl, 3-cyclopentylbut-1-yl, 4-cyclopentylbut-1-yl, 1-cyclopentylbut-2-yl, 2-cyclopentylbut-2-yl, 3-cyclopentylbut-2-yl, 3-cyclopentylbut-2-yl, 4-cyclopentylbut-2-yl, 1-(cyclopentylmethyl)eth-1-yl, 1-(cyclopentylmethyl)-1-(methyl)eth-1-yl, 1-(cyclopentylmethyl)prop-1-yl, cyclohexylmethyl, 1-cyclohexylethyl, 2-cyclohexylethyl, 1-cyclohexylprop-1-yl, 2-cyclohexylprop-1-yl, 3-cyclohexylprop-1-yl, 1-cyclohexylbut-1-yl, 2-cyclohexylbut-1-yl, 3-cyclohexylbut-1-yl, 4-cyclohexylbut-1-yl, 1-cyclohexylbut-2-yl, 2-cyclohexylbut-2-yl, 3-cyclohexylbut-2-yl, 3-cyclohexylbut-2-yl, 4-cyclohexylbut-2-yl, 1-(cyclohexylmethyl)eth-1-yl, 1-(cyclohexylmethyl)-1-(methyl)eth-1-yl, 1-(cyclohexylmethyl)prop-1-yl, cycloheptylmethyl, 1-cycloheptylethyl, 2-cycloheptylethyl, 1-cycloheptylprop-1-yl, 2-cycloheptylprop-1-yl, 3-cycloheptylprop-1-yl, 1-cycloheptylbut-1-yl, 2-cycloheptylbut-1-yl, 3-cycloheptylbut-1-yl, 4-cycloheptylbut-1-yl, 1-cycloheptylbut-2-yl, 2-cycloheptylbut-2-yl, 3-cycloheptylbut-2-yl, 3-cycloheptylbut-2-yl, 4-cycloheptylbut-2-yl, 1-(cycloheptylmethyl)eth-1-yl, 1-(cycloheptylmethyl)-1-(methyl)eth-1-yl, 1-(cycloheptylmethyl)prop-1-yl, cyclooctylmethyl, 1-cyclooctylethyl, 2-cyclooctylethyl, 1-cyclooctylprop-1-yl, 2-cyclooctylprop-1-yl, 3-cyclooctylprop-1-yl, 1-cyclooctylbut-1-yl, 2-cyclooctylbut-1-yl, 3-cyclooctylbut-1-yl, 4-cyclooctylbut-1-yl, 1-cyclooctylbut-2-yl, 2-cyclooctylbut-2-yl, 3-cyclooctylbut-2-yl, 3-cyclooctylbut-2-yl, 4-cyclooctylbut-2-yl, 1-(cyclooctylmethyl)eth-1-yl, 1-(cyclooctylmethyl)-1-(methyl)eth-1-yl or 1-(cyclooctylmethyl)prop-1-yl, preferably cyclopropylmethyl, cyclobutylmethyl, cyclopentylmethyl or cyclohexylmethyl;

C₃-C₈-cycloalkyl-C₁-C₄-alkyl containing a carbonyl or thiocarbonyl ring member: for example cyclobutanon-2-ylmethyl, cyclobutanon-3-ylmethyl, cyclopentanon-2-ylmethyl, cyclopentanon-3-ylmethyl, cyclohexanon-2-ylmethyl, cyclohexanon-4-ylmethyl, cycloheptanon-2-ylmethyl, cyclooctanon-2-ylmethyl, cyclobutanethion-2-ylmethyl, cyclobutanethion-3-ylmethyl, cyclopentanethion-2-ylmethyl, cyclopentanethion-3-ylmethyl, cyclohexanethion-2-ylmethyl, cyclohexanethion-4-ylmethyl, cycloheptanethion-2-ylmethyl, cyclooctanethion-2-ylmethyl, 1-(cyclobutanon-2-yl)ethyl, 1-(cyclobutanon-3-yl)ethyl, 1-(cyclopentanon-2-yl)ethyl, 1-(cyclopentanon-3-yl)ethyl, 1-(cyclohexanon-2-yl)ethyl, 1-(cyclohexanon-4-yl)ethyl, 1-(cycloheptanon-2-yl)ethyl, 1-(cyclooctanon-2-yl)ethyl, 1-(cyclobutanethion-2-yl)ethyl, 1-(cyclobutanethion-3-yl)ethyl, 1-(cyclopentanethion-2-yl)ethyl, 1-(cyclopentanethion-3-yl)ethyl, 1-(cyclohexanethion-2-yl)ethyl, 1-(cyclohexanethion-4-yl)ethyl, 1-(cycloheptanethion-2-yl)ethyl, 1-(cyclooctanethion-2-yl)ethyl, 2-(cyclobutanon-2-yl)ethyl, 2-(cyclobutanon-3-yl)ethyl, 2-(cyclopentanon-2-yl)ethyl, 2-(cyclopentanon-3-yl)ethyl, 2-(cyclohexanon-2-yl)ethyl, 2-(cyclohexanon-4-yl)ethyl, 2-(cycloheptanon-2-yl)ethyl, 2-(cyclooctanon-2-yl)ethyl, 2-(cyclobutanethion-2-yl)ethyl, 2-(cyclobutanethion-3-yl)ethyl, 2-(cyclopentanethion-2-yl)ethyl, 2-(cyclopentanethion-3-yl)ethyl, 2-(cyclohexanethion-2-yl)ethyl, 2-(cyclohexanethion-4-yl)ethyl, 2-(cycloheptanethion-2-yl)ethyl, 2-(cyclooctanethion-2-yl)ethyl, 3-(cyclobutanon-2-yl)propyl, 3-(cyclobutanon-3-yl)propyl, 3-(cyclopentanon-2-yl)propyl, 3-(cyclopentanon-3-yl)propyl, 3-(cyclohexanon-2-yl)propyl, 3-(cyclohexanon-4-yl)propyl, 3-(cycloheptanon-2-yl)propyl, 3-(cyclooctanon-2-yl)propyl, 3-(cyclobutanethion-2-yl)propyl, 3-(cyclobutanethion-3-yl)propyl, 3-(cyclopentanethion-2-yl)propyl, 3-(cyclopentanethion-3-yl)propyl, 3-(cyclohexanethion-2-yl)propyl, 3-(cyclohexanethion-4-yl)propyl, 3-(cycloheptanethion-2-yl)propyl, 3-(cyclooctanethion-2-yl)propyl, 4-(cyclobutanon-2-yl)butyl, 4-(cyclobutanon-3-yl)butyl, 4-(cyclopentanon2-yl)butyl, 4-(cyclopentanon-3-yl)butyl, 4-(cyclohexanon-2-yl)butyl, 4-(cyclohexanon-4-yl)butyl, 4-(cycloheptanon-2-yl)butyl, 4-(cyclooctanon-2-yl)butyl, 4-(cyclobutanethion-2-yl)butyl, 4-(cyclobutanethion-3-yl)butyl, 4-(cyclopentanethion-2-yl)butyl, 4-(cyclopentanethion-3-yl)butyl, 4-(cyclohexanethion-2-yl)butyl, 4-(cyclohexanethion-4-yl)butyl, 4-(cycloheptanethion-2-yl)butyl or 4-(cyclooctanethion-2-yl)butyl, preferably cyclopentanon-2-ylmethyl, cyclohexanon-2-ylmethyl, 2-(cyclopentanon-2-yl)ethyl or 2-(cyclohexanon-2-yl)ethyl.

3- to 7-membered heterocyclyl is to be understood as meaning both saturated, partially or fully unsaturated and aromatic heterocycles having one, two or three heteroatoms selected from the group consisting of nitrogen atoms, oxygen atoms and sulfur atoms. Saturated 3- to 7-membered heterocyclyl may also contain a carbonyl or thiocarbonyl ring member.

Examples of saturated heterocycles which may contain a carbonyl or thiocarbonyl ring member are: oxiranyl, thiiranyl, aziridin-1-yl, aziridin-2-yl, diaziridin-1-yl, diaziridin-3-yl, oxetan-2-yl, oxetan-3-yl, thietan-2-yl, thietan-3-yl, azetidin-1-yl, azetidin-2-yl, azetidin-3-yl, tetrahydrofuran-2-yl, tetrahydrofuran-3-yl, tetrahydrothiophen-2-yl, tetrahydrothiophen-3-yl, pyrrolidin-1-yl, pyrrolidin-2-yl, pyrrolidin-3-yl, 1,3-dioxolan-2-yl, 1,3-dioxolan-4-yl, 1,3-oxathiolan-2-yl, 1,3-oxathiolan-4-yl, 1,3-oxathiolan-5-yl, 1,3-oxazolidin-2-yl, 1,3-oxazolidin-3-yl, 1,3-oxazolidin-4-yl, 1,3-oxazolidin-5-yl, 1,2-oxazolidin-2-yl, 1,2-oxazolidin-3-yl, 1,2-oxazolidin-4-yl, 1,2-oxazolidin-5-yl, 1,3-dithiolan-2-yl, 1,3-dithiolan-4-yl, pyrrolidin-1-yl, pyrrolidin-2-yl, pyrrolidin-5-yl, tetrahydropyrazol-1-yl, tetrahydropyrazol-3-yl, tetrahydropyrazol-4-yl, tetrahydropyran-2-yl, tetrahydropyran-3-yl, tetrahydropyran-4-yl, tetrahydrothiopyran-2-yl, tetrahydrothiopyran-3-yl, tetrahydropyran-4-yl, piperidin-1-yl, piperidin-2-yl, piperidin-3-yl, piperidin-4-yl, 1,3-dioxan-2-yl, 1,3-dioxan-4-yl, 1,3-dioxan-5-yl, 1,4-dioxan-2-yl, 1,3-oxathian-2-yl, 1,3-oxathian-4-yl, 1,3-oxathian-5-yl, 1,3-oxathian-6-yl, 1,4-oxathian-2-yl, 1,4-oxathian-3-yl, morpholin-2-yl, morpholin-3-yl, morpholin-4-yl, hexahydropyridazin-1-yl, hexahydropyridazin-3-yl, hexahydropyridazin-4-yl, hexahydropyrimidin-1-yl, hexahydropyrimidin-2-yl, hexahydropyrimidin-4-yl, hexahydropyrimidin-5-yl, piperazin-1-yl, piperazin-2-yl, piperazin-3-yl, hexahydro-1,3,5-triazin-1-yl, hexahydro-1,3,5-triazin-2-yl, oxepan-2-yl, oxepan-3-yl, oxepan-4-yl, thiepan-2-yl, thiepan-3-yl, thiepan-4-yl, 1,3-dioxepan-2-yl, 1,3-dioxepan-4-yl, 1,3-dioxepan-5-yl, 1,3-dioxepan-6-yl, 1,3-dithiepan-2-yl, 1,4-dioxepan-2-yl, 1,4-dioxepan-7-yl, hexahydroazepin-1-yl, hexahydroazepin-2-yl, hexahydroazepin-3-yl, hexahydroazepin-4-yl, hexahydro-1,3-diazepin-1-yl, hexahydro-1,3-diazepin-2-yl, hexahydro-1,3-diazepin-4-yl, hexahydro-1,4-diazepin-1-yl and hexahydro-1,4-diazepin-2-yl.

Examples of unsaturated heterocycles which may contain a carbonyl or thiocarbonyl ring member are:

dihydrofuran-2-yl, 1,2-oxazolin-3-yl, 1,2-oxazolin-5-yl, 1,3-oxazolin-2-yl.

Examples of aromatic heterocyclyl are the 5- and 6-membered aromatic heterocyclic radicals, for example furyl, such as 2-furyl and 3-furyl, thienyl, such as 2-thienyl and 3-thienyl, pyrrolyl, such as 2-pyrrolyl and 3-pyrrolyl, isoxazolyl, such as 3-isoxazolyl, 4-isoxazolyl and 5-isoxazolyl, isothiazolyl, such as 3-isothiazolyl, 4-isothiazolyl and 5-isothiazolyl, pyrazolyl, such as 3-pyrazolyl, 4-pyrazolyl and 5-pyrazolyl, oxazolyl, such as 2-oxazolyl, 4-oxazolyl and 5-oxazolyl, thiazolyl, such as 2-thiazolyl, 4-thiazolyl and 5-thiazolyl, imidazolyl, such as 2-imidazolyl and 4-imidazolyl, oxadiazolyl, such as 1,2,4-oxadiazol-3-yl, 1,2,4-oxadiazol-5-yl and 1,3,4-oxadiazol-2-yl, thiadiazolyl, such as 1,2,4-thiadiazol-3-yl, 1,2,4-thiadiazol-5-yl and 1,3,4-thiadiazol-2-yl, triazolyl, such as 1,2,4-triazol-1-yl, 1,2,4-triazol-3-yl and 1,2,4-triazol-4-yl, pyridinyl, such as 2-pyridinyl, 3-pyridinyl and 4-pyridinyl, pyridazinyl, such as 3-pyridazinyl and 4-pyridazinyl, pyrimidinyl, such as 2-pyrimidinyl, 4-pyrimidinyl and 5-pyrimidinyl, and furthermore 2-pyrazinyl, 1,3,5-triazin-2-yl and 1,2,4-triazin-3-yl, in particular pyridyl, pyrimidyl, furanyl and thienyl.

Examples of fused-on rings are, in addition to phenyl, the abovementioned heteroaromatic groups, in particular pyridine, pyrazine, pyridazine, pyrimidine, furan, dihydrofuran, thiophene, dihydrothiophene, pyrrole, dihydropyrrole, 1,3-dioxolane, 1,3-dioxolan-2-one, isoxazole, oxazole, oxazolinone, isothiazole, thiazole, pyrazole, pyrazoline, imidazole, imidazolinone, dihydroimidazole, 1,2,3-triazole, 1,1-dioxodihydroisothiazole, dihydro-1,4-dioxine, pyridone, dihydro-1,4-oxazine, dihydro-1,4-oxazin-2-one, dihydro-1,4-oxazin-3-one, dihydro-1,3-oxazine, dihydro-1,3-thiazin-2-one, dihydro-1,4-thiazine, dihydro-1,4-thiazin-2-one, dihydro-1,4-thiazin-3-one, dihydro-1,3-thiazine and dihydro-1,3-thiazin-2-one which for their part may have one, two or three substituents. Examples of suitable substituents on the fused-on ring are the meanings given below for R¹⁵, R¹⁶, R¹⁷ and R¹⁸.

With a view to the use of the 3-arylisothiazoles I as herbicides or desiccants/defoliants, preference is given to those compounds I in which R²≠hydrogen or R⁴≠hydrogen and in which preferably R² and R⁴≠hydrogen. Preference is furthermore given to compounds I where the variables are as defined below, in each case on their own or in combination:

R¹ is C₁-C₄-haloalkyl, in particular trifluoromethyl, C₁-C₄-haloalkoxy, in particular difluoromethoxy, C₁-C₄-alkylsulfonyl, in particular methylsulfonyl, or C₁-C₄-alkylsulfonyloxy, in particular methylsulfonyloxy;

R² is halogen, preferably chlorine, cyano, C₁-C₄-alkyl, preferably methyl, and especially chlorine;

R³ is hydrogen, fluorine or chlorine;

R⁴ is halogen, in particular chlorine, or cyano;

X is a chemical bond, methylene, ethane-1,2-diyl, ethene-1,2-diyl, which may be unsubstituted or may have a substituent selected from the group consisting of C₁-C₄-alkyl, especially methyl, or halogen, especially chlorine, for example 1- or 2-chloroethane-1,2-diyl, 1- or 2-chloroethene-1,2-diyl, 1- or 2-bromoethane-1,2-diyl, 1- or 2-bromoethene-1,2-diyl, 1- or 2-methylethane-1,2-diyl, 1- or 2-methylethene-1,2-diyl, in particular a chemical bond, 1- or 2-chloroethane-1,2-diyl, 1- or 2-chloroethene-1,2-diyl, 1- or 2-bromoethene-1,2-diyl, 1- or 2-methylethene-1,2-diyl. If X is substituted ethane-1,2-diyl, ethene-1,2-diyl, the substituent is preferably located at the carbon atom adjacent to group R⁵;

R⁵ is hydrogen, fluorine, nitro, chlorosulfonyl, —O—Y—R⁷, —O—CO—Y—R⁷, —N(Y—R⁷)(Z—R⁸), —N(Y—R⁷)—SO₂—Z—R⁸, —N(SO₂—Y—R⁷)(SO₂—Z—R⁸), —S—Y—R⁷, —SO₂—N(Y—R⁷)(Z—R⁸), —C(═NOR⁹)—Y—R⁷, —C(═NOR⁹)—O—Y—R⁷, —CO—O—Y—R⁷, PO(O—Y—R⁷) or —CO—N(Y—R⁷)(Z—R⁸), in particular —O—Y—R⁷, —S—Y—R⁷, —N(Y—R⁷)—SO₂—Z—R⁸ or —CO—O—Y—R⁷, and particularly preferably —O—Y—R⁷.

The variables R⁷, R⁸, R⁹, Y and Z mentioned in the definition of the variable R⁵ are preferably as defined below:

Y, Z independently of one another are a chemical bond or methylene;

R⁷, R⁸ independently of one another are hydrogen, C₁-C₄-haloalkyl, C₂-C₆-alkenyl, C₂-C₆-alkynyl, —CH(R¹⁰)(R¹¹), C₁-C₄-alkoxy-C₁-C₄-alkyl, —C(R¹⁰)(R¹¹)—N(R¹²)R¹³, —C(R¹⁰)(R¹¹)—CO—OR¹², —C(R¹⁰)(R¹¹)—CO—N(R¹²)R^{13, C}₃-C₈-cycloalkyl or phenyl, where the cycloalkyl and the phenyl ring may be unsubstituted or may carry one or two substituents, in each case selected from the group consisting of cyano, nitro, halogen, C₁-C₄-alkyl, C₁-C₄-alkoxy, C₁-C₄-alkylsulfonyl, (C₁-C₄-alkyl)carbonyl, (C₁-C₄-alkyl)carbonyloxy and (C₁-C₄-alkoxy)carbonyl;

in particular hydrogen, C₁-C₆-haloalkyl, C₁-C₄-alkoxy-C₁-C₄-alkyl, C₂-C₆-alkenyl, C₂-C₆-alkynyl, —CH(R¹⁰)(R¹¹), —C(R¹⁰)(R¹¹)—CO—OR¹², —C(R¹⁰)(R¹¹)—CO—N(R¹²)R¹³, phenyl or C₃-C₈-cycloalkyl, particularly preferably hydrogen, C₁-C₆-alkyl, C₁-C₄-alkoxy-C₁-C₄-alkyl, C₂-C₆-alkenyl, C₂-C₆-alkynyl, —C(R¹¹)(R¹²)—CO—OR¹³ or C₃-C₈-cycloalkyl.

Here, the variables R¹⁰, R¹¹, R¹², and R¹³ independently of one another are preferably as defined below:

R¹⁰ is hydrogen or C₁-C₄-alkyl, especially methyl;

R¹¹is hydrogen or methyl;

R¹², R¹³ independently of one another are hydrogen, C₁-C₆-alkyl, C₂-C₆-alkenyl, C₂-C₆-alkynyl, C₃-C₈-cycloalkyl, C₃-C₈-cycloalkyl-C₁-C₄-alkyl, or C₁-C₄-alkoxy-C₁-C₄-alkyl, in particular hydrogen or C₁-C₆-alkyl;

R⁹ is C₁-C₆-alkyl, C₁-C₄-alkoxycarbonyl-C₁-C₄-alkyl, C₂-C₆-alkenyl, in particular methyl or ethyl.

Compounds I in which Q=C—H and the variables X, R³, R⁴ and R⁵ are as defined above are hereinbelow referred to as compounds IA. Compounds of the formula IA are particularly preferred according to the invention. Compounds where Q=N are hereinbelow referred to as compounds IB.

In formula I, R⁴ and XR⁵ or XR⁵ and R⁶ can also form a 3- or 4-membered chain which, in addition to carbon, may have 1, 2 or 3 heteroatoms selected from the group consisting of nitrogen, oxygen and sulfur atoms, which may be unsubstituted or may for its part carry one, two or three substituents and whose members may also include one or two not adjacent carbonyl, thiocarbonyl or sulfonyl groups. Such compounds are hereinbelow referred to as compounds IC and compounds ID, respectively.

Among these, preference is given to compounds I in which R⁴ together with X—R⁵ in formula I is a chain of the formula: —O—C(R¹⁵,R¹⁶)—CO—N(R¹⁷)—, —S—C(R¹⁵,R¹⁶)—CO—N(R¹⁷)—, —N═C(R¹⁸)—O— or —N═C(R¹⁸)—S— (compounds IC) in which the variables R¹⁵ to R¹⁸ are as defined below:

R¹⁵, R¹⁶ independently of one another are hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, phenyl or phenyl-C₁-C₄-alkyl;

R¹⁷ is hydrogen, hydroxyl, C₁-C₆-alkyl, C₁-C₆-haloalkyl, C₂-C₆-alkenyl, C₂-C₆-haloalkenyl, C₂-C₆-alkynyl, C₁-C₄-alkoxy, C₁-C₄-haloalkoxy, C₃-C₆-alkenyloxy, C₃-C₆-alkynyloxy, C₁-C₄-alkylsulfonyl, C₁-C₄-haloalkylsulfonyl, C₁-C₄-alkylcarbonyl, C₁-C₄-haloalkylcarbonyl, C₁-C₄-alkoxycarbonyl, C₁-C₄-alkoxy-C₁-C₄-alkyl, C₁-C₄-alkoxycarbonyl-C₁-C₄-alkyl, C₁-C₄-alkoxycarbonyl-C₁-C₄-alkoxy, di(C₁-C₄-alkyl)aminocarbonyl, di(C₁-C₄-alkyl)aminocarbonyl-C₁-C₄-alkyl, di(C₁-C₄-alkyl)aminocarbonyl-C₁-C₄-alkoxy, phenyl, phenyl-C₁-C₄-alkyl, C₃-C₈-cycloalkyl, C₃-C₈-cycloalkyl-C₁-C₄-alkyl, 3-, 4-, 5-, 6- or 7-membered, preferably 5- or 6-membered, preferably saturated heterocyclyl which has one or two, preferably one, ring heteroatom selected from the group consisting of oxygen, nitrogen and sulfur;

R¹⁸ is hydrogen, halogen, cyano, amino, C₁-C₆-alkyl, C₁-C₆-haloalkyl, C₂-C₆-alkenyl, C₂-C₆-haloalkenyl, C₂-C₆-alkynyl, C₁-C₄-alkoxy, C₁-C₄-haloalkoxy, C₃-C₆-alkenyloxy, C₃-C₆-alkynyloxy, C₁-C₄-alkylamino, di(C₁-C₄-alkyl)amino, C₁-C₄-haloalkoxy, C₁-C₄-alkylthio, C₁-C₄-haloalkylthio, C₁-C₄-alkylsulfinyl, C₁-C₄-haloalkylsulfinyl, C₁-C₄-alkylsulfonyl, C₁-C₄-haloalkylsulfonyl, C₁-C₄-alkylcarbonyl, C₁-C₄-haloalkylcarbonyl, C₁-C₄-alkoxy-C₁-C₄-alkyl, C₁-C₄-alkoxycarbonyl, C₁-C₄-alkoxycarbonyl-C₁-C₄-alkyl, C₁-C₄-alkoxycarbonyl-C₁-C₄-alkoxy, C₁-C₄-alkoxycarbonyl-C₁-C₄-alkylthio, di(C₁-C₄-alkyl)aminocarbonyl, di(C₁-C₄-alkyl)aminocarbonyl-C₁-C₄-alkyl, di(C₁-C₄-alkyl)aminocarbonyl-C₁-C₄-alkoxy, di(C₁-C₄-alkyl)aminocarbonyl-C₁-C₄-alkylthio, C₃-C₈-cycloalkyl, phenyl, phenyl-C₁-C₄-alkyl, C₃-C₈-cycloalkyl-C₁-C₄-alkyl, 3-, 4-, 5-, 6- or 7-membered, preferably 5- or 6-membered, preferably saturated heterocyclyl which contains one or two, preferably one, ring heteroatom selected from the group consisting of oxygen, nitrogen and sulfur.

The variables R¹⁵ to R¹⁸ are preferably as defined below:

R¹⁵, R¹⁶ independently of one another are hydrogen or methyl;

R¹⁷ is hydrogen, hydroxyl, C₁-C₄-alkyl, C₁-C₄-haloalkyl, C₂-C₆-alkenyl, C₂-C₆-alkynyl, C₁-C₄-alkoxy, C₁-C₄-haloalkoxy, C₃-C₆-alkenyloxy, C₃-C₆-alkynyloxy, C₁-C₄-alkoxycarbonyl-C₁-C₄-alkyl, C₁-C₄-alkoxycarbonyl-C₁-C₄-alkoxy, C₃-C₈-cycloalkyl, C₃-C₈-cycloalkyl-C₁-C₄-alkyl or phenyl-C₁-C₄-alkyl or 3-, 4-, 5- or 6-membered, preferably 5- or 6-membered, preferably saturated heterocyclyl which has one ring heteroatom selected from the group consisting of oxygen, nitrogen and sulfur;

R¹⁸ is hydrogen, halogen, amino, C₁-C₆-alkyl, C₁-C₆-haloalkyl, C₂-C₆-alkenyl, C₂-C₆-haloalkenyl, C₂-C₆-alkynyl, C₁-C₄-alkoxy, C₃-C₆-alkenyloxy, C₃-C₆-alkynyloxy, C₁-C₄-alkylamino, di(C₁-C₄-alkyl)amino, C₁-C₄-alkylthio, C₁-C₄-alkoxycarbonyl-C₁-C₄-alkyl, C₁-C₄-alkoxycarbonyl-C₁-C₄-alkoxy, C₁-C₄-alkoxycarbonyl-C₁-C₄-alkylthio, C₃-C₈-cycloalkyl, phenyl, phenyl-C₁-C₄-alkyl, C₃-C₈-cycloalkyl-C₁-C₄-alkyl, 3-, 4-, 5- or 6-membered, preferably 5- or 6-membered, preferably saturated heterocyclyl which has one ring heteroatom selected from the group consisting of oxygen, nitrogen and sulfur.

In these compounds, Q and R³ have the meanings mentioned above, where Q is in particular CH and R³ has in particular the meanings given as being preferred.

Among the compounds IC, particular preference is given to those compounds in which R⁴ together with X—R⁵ is a chain of the formula —O—CH(R¹⁵)—CO—N(R¹⁷)— or —S—CH(R¹⁵)—CO—N(R¹⁷)—. R¹⁵ and R¹⁷ have in particular the meanings given as being preferred. Among these, very particular preference is given to the compounds IC in which the nitrogen atom of the chain —O—CH(R¹⁵)—CO—N(R¹⁷)— or —S—CH(R¹⁵)—CO—N(R¹⁷)— is attached to the carbon atom of the phenyl ring in the formula I which is adjacent to the group Q (meta-position with respect to the isothiazolyl group).

Preference is furthermore given to compounds I in which Q is a group C—R⁶ and R⁶ together with X—R⁵ is a chain of the formula: —O—C(R¹⁵,R¹⁶)—CO—N(R¹⁷)—, —S—C(R¹⁵,R¹⁶)—CO—N(R¹⁷)—, —N═C(R¹⁸)—O— or —N═C(R¹⁸)—S— (compounds ID) in which the variables R¹⁵ to R¹⁸ are as defined above and have in particular the meanings given as being preferred. Among these, preference is given to those compounds in which R⁶ together with X—R⁵ is a chain of the formula —N═C(R¹⁸)—O— or —N═C(R¹⁸)—S—. In these compounds, R³ and R⁴ have the meanings mentioned above, in particular those given as being preferred.

Particular preference is given to the compounds of the formula IAa (compounds IA where Q=CH, R¹=CF₃ and R²=Cl) in which the variables R³, R⁴ and X—R⁵ together have the meanings given in each case in one row of Table 1 (compounds IAa.1-IAa.776).

TABLE 1

| | |
|---|---|
| | (IAa) |
| 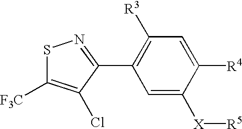 | |

| No. | R³ | R⁴ | X-R⁵ | |
|---|---|---|---|---|
| 1 | F | Cl | H | |
| 2 | F | Cl | F | |
| 3 | F | Cl | CH₃ | |
| 4 | F | Cl | NO₂ | |
| 5 | F | Cl | NH₂ | |
| 6 | F | Cl | OH | |
| 7 | F | Cl | OCH₃ | |
| 8 | F | Cl | OCH(CH₃)₂ | |
| 9 | F | Cl | O—CH₂CH═CH₂ | |
| 10 | F | Cl | O—CH₂C≡CH | |
| 11 | F | Cl | O—CH(CH₃)C≡CH | |
| 12 | F | Cl | O-cyclopentyl | |
| 13 | F | Cl | OCH₂COOH | |
| 14 | F | Cl | OCH₂COO—CH₃ | |
| 15 | F | Cl | OCH₂COO—CH₂CH₃ | |
| 16 | F | Cl | OCH₂COO—CH₂CH═CH₂ | |
| 17 | F | Cl | OCH₂COO—CH₂C≡CH | |
| 18 | F | Cl | OCH₂COO—CH₂CH₂OCH₃ | |
| 19 | F | Cl | OCH₂CONH—CH₃ | |
| 20 | F | Cl | OCH₂CON(CH₃)₂ | |
| 21 | F | Cl | OCH(CH₃)COOH | |
| 22 | F | Cl | OCH(CH₃)COO—CH₃ | |
| 23 | F | Cl | OCH(CH₃)COO—CH₂CH₃ | |
| 24 | F | Cl | OCH(CH₃)COO—CH₂CH═CH₂ | |
| 25 | F | Cl | OCH(CH₃)COO—CH₂C≡CH | |
| 26 | F | Cl | OCH(CH₃)COO—CH₂CH₂OCH₃ | |
| 27 | F | Cl | OCH(CH₃)CONH—CH₃ | |
| 28 | F | Cl | OCH(CH₃)CON(CH₃)₂ | |
| 29 | F | Cl | OC(CH₃)₂COO—CH₃ | |
| 30 | F | Cl | OC(CH₃)₂COO—CH₂CH═CH₂ | |
| 31 | F | Cl | SH | |
| 32 | F | Cl | SCH₃ | |
| 33 | F | Cl | SCH(CH₃)₂ | |
| 34 | F | Cl | S—CH₂CH═CH₂ | |
| 35 | F | Cl | S—CH₂C≡CH | |
| 36 | F | Cl | S—CH(CH₃)C≡CH | |
| 37 | F | Cl | S-cyclopentyl | |
| 38 | F | Cl | SCH₂COOH | |
| 39 | F | Cl | SCH₂COO—CH₃ | |
| 40 | F | Cl | SCH₂COO—CH₂CH₃ | |
| 41 | F | Cl | SCH₂COO—CH₂CH═CH₂ | |
| 42 | F | Cl | SCH₂COO—CH₂C≡CH | |
| 43 | F | Cl | SCH₂COO—CH₂CH₂OCH₃ | |
| 44 | F | Cl | SCH₂CONH—CH₃ | |
| 45 | F | Cl | SCH₂CON(CH₃)₂ | |
| 46 | F | Cl | SCH(CH₃)COOH | |
| 47 | F | Cl | SCH(CH₃)COO—CH₃ | |
| 48 | F | Cl | SCH(CH₃)COO—CH₂CH₃ | |
| 49 | F | Cl | SCH(CH₃)COO—CH₂CH═CH₂ | |
| 50 | F | Cl | SCH(CH₃)COO—CH₂C≡CH | |
| 51 | F | Cl | SCH(CH₃)COO—CH₂CH₂OCH₃ | |
| 52 | F | Cl | SCH(CH₃)CONH—CH₃ | |
| 53 | F | Cl | SCH(CH₃)CON(CH₃)₂ | |
| 54 | F | Cl | SC(CH₃)₂COO—CH₃ | |
| 55 | F | Cl | SC(CH₃)₂COO—CH₂CH═CH₂ | |
| 56 | F | Cl | COOH | |
| 57 | F | Cl | COOCH₃ | |
| 58 | F | Cl | COOCH₂CH₃ | |
| 59 | F | Cl | COOCH(CH₃)₂ | |
| 60 | F | Cl | COO—CH₂CH═CH₂ | |
| 61 | F | Cl | COO—CH₂C≡CH | |
| 62 | F | Cl | COO-cyclopentyl | |
| 63 | F | Cl | COO—CH₂COO—CH₃ | |
| 64 | F | Cl | COO—CH₂COO—CH₂CH₃ | |
| 65 | F | Cl | COO—CH₂COO—CH₂CH═CH₂ | |
| 66 | F | Cl | COO—CH₂COO—CH₂C≡CH | |
| 67 | F | Cl | COO—CH₂COO—CH₂CH₂OCH₃ | |
| 68 | F | Cl | COO—CH(CH₃)COO—CH₃ | |
| 69 | F | Cl | COO—CH(CH₃)COO—CH₂CH₃ | |
| 70 | F | Cl | COO—CH(CH₃)COO—CH₂CH═CH₂ | |
| 71 | F | Cl | COO—CH(CH₃)COO—CH₂C≡CH | |
| 72 | F | Cl | COO—CH(CH₃)COO—CH₂CH₂OCH₃ | |
| 73 | F | Cl | COO—C(CH₃)₂COO—CH₃ | |
| 74 | F | Cl | COO—C(CH₃)₂COO—CH₂CH₃ | |
| 75 | F | Cl | COO—C(CH₃)₂COO—CH₂CH═CH₂ | |
| 76 | F | Cl | COO—C(CH₃)₂COO—CH₂C≡CH | |
| 77 | F | Cl | COO—C(CH₃)₂COO—CH₂CH₂OCH₃ | |
| 78 | F | Cl | CONH₂ | |
| 79 | F | Cl | CONHCH₃ | |
| 80 | F | Cl | CON(CH₃)₂ | |
| 81 | F | Cl | CONH—CH₂COO—CH₃ | |
| 82 | F | Cl | CONH—CH₂COO—CH₂CH═CH₂ | |
| 83 | F | Cl | CONH—CH₂COO—CH₂CH₂OCH₃ | |
| 84 | F | Cl | CONH—CH(CH₃)COO—CH₃ | |
| 85 | F | Cl | CONH—CH(CH₃)COO—CH₂CH═CH₂ | |
| 86 | F | Cl | CONH—CH(CH₃)COO—CH₂CH₂OCH₃ | |
| 87 | F | Cl | CON(CH₃)—CH₂COO—CH₃ | |
| 88 | F | Cl | CON(CH₃)—CH₂COO—CH₂CH═CH₂ | |
| 89 | F | Cl | CON(CH₃)—CH₂COO—CH₂CH₂OCH₃ | |
| 90 | F | Cl | C(═N—OCH₃)O—CH₃ | |
| 91 | F | Cl | C(═N—OCH₃)O—CH₂—COOCH₃ | |
| 92 | F | Cl | C(═N—OCH₃)O—CH₂—COO-phenyl | |
| 93 | F | Cl | C(═N—OCH₃)O—CH(CH₃)—COOCH₃ | |
| 94 | F | Cl | CH═C(Cl)COO—CH₃ | |
| 95 | F | Cl | CH═C(Cl)COO—CH₂CH₃ | |
| 96 | F | Cl | CH═C(Cl)COO—CH₂CH═CH₂ | |
| 97 | F | Cl | CH═C(Cl)COO—CH₂COOCH₃ | |
| 98 | F | Cl | CH═C(Cl)COO—CH(CH₃)COOCH₃ | |
| 99 | F | Cl | CH═C(Cl)CON(CH₃)₂ | |
| 100 | F | Cl | CH═C(Cl)CON(CH₃)—CH₂COOCH₃ | |
| 101 | F | Cl | CH═C(Cl)CONH—CH(CH₃)COOCH₃ | |
| 102 | F | Cl | CH═C(Br)COO—CH₃ | |
| 103 | F | Cl | CH═C(Br)COO—CH₂CH₃ | |
| 104 | F | Cl | CH═C(CH₃)COO—CH₃ | |
| 105 | F | Cl | CH═C(CH₃)COO—CH₂CH₃ | |
| 106 | F | Cl | CH₂—CH(Cl)—COO—CH₃ | |
| 107 | F | Cl | CH₂—CH(Cl)—COO—CH₂CH₃ | |
| 108 | F | Cl | CHO | |
| 109 | F | Cl | CH═N—OCH₃ | |
| 110 | F | Cl | CH═N—OCH₂CH₃ | |
| 111 | F | Cl | CH═N—OCH(CH₃)COOCH₃ | |
| 112 | F | Cl | SO₂Cl | |
| 113 | F | Cl | SO₂NH₂ | |
| 114 | F | Cl | SO₂NHCH₃ | |
| 115 | F | Cl | SO₂N(CH₃)₂ | |
| 116 | F | Cl | NH—CH₂C≡CH | |
| 117 | F | Cl | NHCH(CH₃)COOCH₃ | |
| 118 | F | Cl | N(CH₃)—CH₂C≡CH | |
| 119 | F | Cl | NH(SO₂CH₃) | |
| 120 | F | Cl | N(CH₃)(SO₂CH₃) | |
| 121 | F | Cl | N(SO₂CH₃)₂ | |
| 122 | F | CN | H | |
| 123 | F | CN | F | |
| 124 | F | CN | CH₃ | |
| 125 | F | CN | NO₂ | |
| 126 | F | CN | NH₂ | |
| 127 | F | CN | OH | |
| 128 | F | CN | OCH₃ | |
| 129 | F | CN | OCH(CH₃)₂ | |
| 130 | F | CN | O—CH₂CH═CH₂ | |
| 131 | F | CN | O—CH₂C≡CH | |
| 132 | F | CN | O—CH(CH₃)C≡CH | |
| 133 | F | CN | O-cyclopentyl | |
| 134 | F | CN | OCH₂COOH | |
| 135 | F | CN | OCH₂COO—CH₃ | |
| 136 | F | CN | OCH₂COO—CH₂CH₃ | |
| 137 | F | CN | OCH₂COO—CH₂CH═CH₂ | |
| 138 | F | CN | OCH₂COO—CH₂C≡CH | |
| 139 | F | CN | OCH₂COO—CH₂CH₂OCH₃ | |
| 140 | F | CN | OCH₂CONH—CH₃ | |
| 141 | F | CN | OCH₂CON(CH₃)₂ | |
| 142 | F | CN | OCH(CH₃)COOH | |
| 143 | F | CN | OCH(CH₃)COO—CH₃ | |
| 144 | F | CN | OCH(CH₃)COO—CH₂CH₃ | |
| 145 | F | CN | OCH(CH₃)COO—CH₂CH═CH₂ | |
| 146 | F | CN | OCH(CH₃)COO—CH₂C≡CH | |
| 147 | F | CN | OCH(CH₃)COO—CH₂CH₂OCH₃ | |
| 148 | F | CN | OCH(CH₃)CONH—CH₃ | |
| 149 | F | CN | OCH(CH₃)CON(CH₃)₂ | |
| 150 | F | CN | OC(CH₃)₂COO—CH₃ | |
| 151 | F | CN | OC(CH₃)₂COO—CH₂CH═CH₂ | |
| 152 | F | CN | SH | |
| 153 | F | CN | SCH₃ | |
| 154 | F | CN | SCH(CH₃)₂ | |
| 155 | F | CN | S—CH₂CH═CH₂ | |
| 156 | F | CN | S—CH₂C≡CH | |
| 157 | F | CN | S—CH(CH₃)C≡CH | |
| 158 | F | CN | S-cyclopentyl | |
| 159 | F | CN | SCH₂COOH | |
| 160 | F | CN | SCH₂COO—CH₃ | |
| 161 | F | CN | SCH₂COO—CH₂CH₃ | |
| 162 | F | CN | SCH₂COO—CH₂CH═CH₂ | |
| 163 | F | CN | SCH₂COO—CH₂C≡CH | |
| 164 | F | CN | SCH₂COO—CH₂CH₂OCH₃ | |
| 165 | F | CN | SCH₂CONH—CH₃ | |
| 166 | F | CN | SCH₂CON(CH₃)₂ | |
| 167 | F | CN | SCH(CH₃)COOH | |
| 168 | F | CN | SCH(CH₃)COO—CH₃ | |
| 169 | F | CN | SCH(CH₃)COO—CH₂CH₃ | |
| 170 | F | CN | SCH(CH₃)COO—CH₂CH═CH₂ | |
| 171 | F | CN | SCH(CH₃)COO—CH₂C≡CH | |
| 172 | F | CN | SCH(CH₃)COO—CH₂CH₂OCH₃ | |
| 173 | F | CN | SCH(CH₃)CONH—CH₃ | |
| 174 | F | CN | SCH(CH₃)CON(CH₃)₂ | |
| 175 | F | CN | SC(CH₃)₂COO—CH₃ | |
| 176 | F | CN | SC(CH₃)₂COO—CH₂CH═CH₂ | |
| 177 | F | CN | COOH | |
| 178 | F | CN | COOCH₃ | |
| 179 | F | CN | COOCH₂CH₃ | |
| 180 | F | CN | COOCH(CH₃)₂ | |
| 181 | F | CN | COO—CH₂CH═CH₂ | |
| 182 | F | CN | COO—CH₂C≡CH | |
| 183 | F | CN | COO-cyclopentyl | |
| 184 | F | CN | COO—CH₂COO—CH₃ | |
| 185 | F | CN | COO—CH₂COO—CH₂CH₃ | |
| 186 | F | CN | COO—CH₂COO—CH₂CH═CH₂ | |
| 187 | F | CN | COO—CH₂COO—CH₂C≡CH | |
| 188 | F | CN | COO—CH₂COO—CH₂CH₂OCH₃ | |
| 189 | F | CN | COO—CH(CH₃)COO—CH₃ | |
| 190 | F | CN | COO—CH(CH₃)COO—CH₂CH₃ | |
| 191 | F | CN | COO—CH(CH₃)COO—CH₂CH═CH₂ | |
| 192 | F | CN | COO—CH(CH₃)COO—CH₂C≡CH | |
| 193 | F | CN | COO—CH(CH₃)COO—CH₂CH₂OCH₃ | |
| 194 | F | CN | COO—C(CH₃)₂COO—CH₃ | |
| 195 | F | CN | COO—C(CH₃)₂COO—CH₂CH₃ | |
| 196 | F | CN | COO—C(CH₃)₂COO—CH₂CH═CH₂ | |
| 197 | F | CN | COO—C(CH₃)₂COO—CH₂C≡CH | |
| 198 | F | CN | COO—C(CH₃)₂COO—CH₂CH₂OCH₃ | |
| 199 | F | CN | CONH₂ | |
| 200 | F | CN | CONHCH₃ | |
| 201 | F | CN | CON(CH₃)₂ | |
| 202 | F | CN | CONH—CH₂COO—CH₃ | |
| 203 | F | CN | CONH—CH₂COO—CH₂CH═CH₂ | |
| 204 | F | CN | CONH—CH₂COO—CH₂CH₂OCH₃ | |
| 205 | F | CN | CONH—CH(CH₃)COO—CH₃ | |
| 206 | F | CN | CONH—CH(CH₃)COO—CH₂CH═CH₂ | |
| 207 | F | CN | CONH—CH(CH₃)COO—CH₂CH₂OCH₃ | |
| 208 | F | CN | CON(CH₃)—CH₂COO—CH₃ | |
| 209 | F | CN | CON(CH₃)—CH₂COO—CH₂CH═CH₂ | |
| 210 | F | CN | CON(CH₃)—CH₂COO—CH₂CH₂OCH₃ | |
| 211 | F | CN | C(═N—OCH₃)O—CH₃ | |
| 212 | F | CN | C(═N—OCH₃)O—CH₂—COOCH₃ | |
| 213 | F | CN | C(═N—OCH₃)O—CH₂—COO-phenyl | |
| 214 | F | CN | C(═N—OCH₃)O—CH(CH₃)—COOCH₃ | |
| 215 | F | CN | CH═C(Cl)COO—CH₃ | |
| 216 | F | CN | CH═C(Cl)COO—CH₂CH₃ | |
| 217 | F | CN | CH═C(Cl)COO—CH₂CH═CH₂ | |
| 218 | F | CN | CH═C(Cl)COO—CH₂COOCH₃ | |
| 219 | F | CN | CH═C(Cl)COO—CH(CH₃)COOCH₃ | |
| 220 | F | CN | CH═C(Cl)CON(CH₃)₂ | |
| 221 | F | CN | CH═C(Cl)CON(CH₃)—CH₂COOCH₃ | |
| 222 | F | CN | CH═C(Cl)CONH—CH(CH₃)COOCH₃ | |
| 223 | F | CN | CH═C(Br)COO—CH₃ | |
| 224 | F | CN | CH═C(Br)COO—CH₂CH₃ | |
| 225 | F | CN | CH═C(CH₃)COO—CH₃ | |
| 226 | F | CN | CH═C(CH₃)COO—CH₂CH₃ | |
| 227 | F | CN | CH₂—CH(Cl)—COO—CH₃ | |
| 228 | F | CN | CH₂—CH(Cl)—COO—CH₂CH₃ | |
| 229 | F | CN | CHO | |
| 230 | F | CN | CH═N—OCH₃ | |
| 231 | F | CN | CH═N—OCH₂CH₃ | |
| 232 | F | CN | CH═N—OCH(CH₃)COOCH₃ | |
| 233 | F | CN | SO₂Cl | |
| 234 | F | CN | SO₂NH₂ | |
| 235 | F | CN | SO₂NHCH₃ | |
| 236 | F | CN | SO₂N(CH₃)₂ | |
| 237 | F | CN | NH—CH₂C≡CH | |
| 238 | F | CN | NHCH(CH₃)COOCH₃ | |
| 239 | F | CN | N(CH₃)—CH₂C≡CH | |
| 240 | F | CN | NH(SO₂CH₃) | |
| 241 | F | CN | N(CH₃)(SO₂CH₃) | |
| 242 | F | CN | N(SO₂CH₃)₂ | |
| 243 | Cl | Cl | H | |
| 244 | Cl | Cl | F | |
| 245 | Cl | Cl | CH₃ | |
| 246 | Cl | Cl | NO₂ | |
| 247 | Cl | Cl | NH₂ | |
| 248 | Cl | Cl | OH | |
| 249 | Cl | Cl | OCH₃ | |
| 250 | Cl | Cl | OCH(CH₃)₂ | |
| 251 | Cl | Cl | O—CH₂CH═CH₂ | |
| 252 | Cl | Cl | O—CH₂C≡CH | |
| 253 | Cl | Cl | O—CH(CH₃)C≡CH | |
| 254 | Cl | Cl | O-cyclopentyl | |
| 255 | Cl | Cl | OCH₂COOH | |
| 256 | Cl | Cl | OCH₂COO—CH₃ | |
| 257 | Cl | Cl | OCH₂COO—CH₂CH₃ | |
| 258 | Cl | Cl | OCH₂COO—CH₂CH═CH₂ | |
| 259 | Cl | Cl | OCH₂COO—CH₂C≡CH | |
| 260 | Cl | Cl | OCH₂COO—CH₂CH₂OCH₃ | |
| 261 | Cl | Cl | OCH₂CONH—CH₃ | |
| 262 | Cl | Cl | OCH₂CON(CH₃)₂ | |
| 263 | Cl | Cl | OCH(CH₃)COOH | |
| 264 | Cl | Cl | OCH(CH₃)COO—CH₃ | |
| 265 | Cl | Cl | OCH(CH₃)COO—CH₂CH₃ | |
| 266 | Cl | Cl | OCH(CH₃)COO—CH₂CH═CH₂ | |
| 267 | Cl | Cl | OCH(CH₃)COO—CH₂C≡CH | |
| 268 | Cl | Cl | OCH(CH₃)COO—CH₂CH₂OCH₃ | |
| 269 | Cl | Cl | OCH(CH₃)CONH—CH₃ | |
| 270 | Cl | Cl | OCH(CH₃)CON(CH₃)₂ | |
| 271 | Cl | Cl | OC(CH₃)₂COO—CH₃ | |
| 272 | Cl | Cl | OC(CH₃)₂COO—CH₂CH═CH₂ | |
| 273 | Cl | Cl | SH | |
| 274 | Cl | Cl | SCH₃ | |
| 275 | Cl | Cl | SCH(CH₃)₂ | |
| 276 | Cl | Cl | S—CH₂CH═CH₂ | |
| 277 | Cl | Cl | S—CH₂C≡CH | |
| 278 | Cl | Cl | S—CH(CH₃)C≡CH | |
| 279 | Cl | Cl | S-cyclopentyl | |
| 280 | Cl | Cl | SCH₂COOH | |
| 281 | Cl | Cl | SCH₂COO—CH₃ | |
| 282 | Cl | Cl | SCH₂COO—CH₂CH₃ | |
| 283 | Cl | Cl | SCH₂COO—CH₂CH═CH₂ | |
| 284 | Cl | Cl | SCH₂COO—CH₂C≡CH | |
| 285 | Cl | Cl | SCH₂COO—CH₂CH₂OCH₃ | |
| 286 | Cl | Cl | SCH₂CONH—CH₃ | |
| 287 | Cl | Cl | SCH₂CON(CH₃)₂ | |
| 288 | Cl | Cl | SCH(CH₃)COOH | |
| 289 | Cl | Cl | SCH(CH₃)COO—CH₃ | |
| 290 | Cl | Cl | SCH(CH₃)COO—CH₂CH₃ | |
| 291 | Cl | Cl | SCH(CH₃)COO—CH₂CH═CH₂ | |
| 292 | Cl | Cl | SCH(CH₃)COO—CH₂C≡CH | |
| 293 | Cl | Cl | SCH(CH₃)COO—CH₂CH₂OCH₃ | |
| 294 | Cl | Cl | SCH(CH₃)CONH—CH₃ | |
| 295 | Cl | Cl | SCH(CH₃)CON(CH₃)₂ | |
| 296 | Cl | Cl | SC(CH₃)₂COO—CH₃ | |
| 297 | Cl | Cl | SC(CH₃)₂COO—CH₂CH═CH₂ | |
| 298 | Cl | Cl | COOH | |
| 299 | Cl | Cl | COOCH₃ | |
| 300 | Cl | Cl | COOCH₂CH₃ | |
| 301 | Cl | Cl | COOCH(CH₃)₂ | |
| 302 | Cl | Cl | COO—CH₂CH═CH₂ | |
| 303 | Cl | Cl | COO—CH₂C≡CH | |
| 304 | Cl | Cl | COO-cyclopentyl | |
| 305 | Cl | Cl | COO—CH₂COO—CH₃ | |
| 306 | Cl | Cl | COO—CH₂COO—CH₂CH₃ | |
| 307 | Cl | Cl | COO—CH₂COO—CH₂CH═CH₂ | |
| 308 | Cl | Cl | COO—CH₂COO—CH₂C≡CH | |
| 309 | Cl | Cl | COO—CH₂COO—CH₂CH₂OCH₃ | |
| 310 | Cl | Cl | COO—CH(CH₃)COO—CH₃ | |
| 311 | Cl | Cl | COO—CH(CH₃)COO—CH₂CH₃ | |
| 312 | Cl | Cl | COO—CH(CH₃)COO—CH₂CH═CH₂ | |
| 313 | Cl | Cl | COO—CH(CH₃)COO—CH₂C≡CH | |
| 314 | Cl | Cl | COO—CH(CH₃)COO—CH₂CH₂OCH₃ | |
| 315 | Cl | Cl | COO—C(CH₃)₂COO—CH₃ | |
| 316 | Cl | Cl | COO—C(CH₃)₂COO—CH₂CH₃ | |
| 317 | Cl | Cl | COO—C(CH₃)₂COO—CH₂CH═CH₂ | |
| 318 | Cl | Cl | COO—C(CH₃)₂COO—CH₂C≡CH | |
| 319 | Cl | Cl | COO—C(CH₃)₂COO—CH₂CH₂OCH₃ | |
| 320 | Cl | Cl | CONH₂ | |
| 321 | Cl | Cl | CONHCH₃ | |
| 322 | Cl | Cl | CON(CH₃)₂ | |
| 323 | Cl | Cl | CONH—CH₂COO—CH₃ | |
| 324 | Cl | Cl | CONH—CH₂COO—CH₂CH═CH₂ | |
| 325 | Cl | Cl | CONH—CH₂COO—CH₂CH₂OCH₃ | |
| 326 | Cl | Cl | CONH—CH(CH₃)COO—CH₃ | |
| 327 | Cl | Cl | CONH—CH(CH₃)COO—CH₂CH═CH₂ | |
| 328 | Cl | Cl | CONH—CH(CH₃)COO—CH₂CH₂OCH₃ | |
| 329 | Cl | Cl | CON(CH₃)—CH₂COO—CH₃ | |
| 330 | Cl | Cl | CON(CH₃)—CH₂COO—CH₂CH═CH₂ | |
| 331 | Cl | Cl | CON(CH₃)—CH₂COO—CH₂CH₂OCH₃ | |
| 332 | Cl | Cl | C(═N—OCH₃)O—CH₃ | |
| 333 | Cl | Cl | C(═N—OCH₃)O—CH₂—COOCH₃ | |
| 334 | Cl | Cl | C(═N—OCH₃)O—CH₂—COO-phenyl | |
| 335 | Cl | Cl | C(═N—OCH₃)O—CH(CH₃)—COOCH₃ | |
| 336 | Cl | Cl | CH═C(Cl)COO—CH₃ | |
| 337 | Cl | Cl | CH═C(Cl)COO—CH₂CH₃ | |
| 338 | Cl | Cl | CH═C(Cl)COO—CH₂CH═CH₂ | |
| 339 | Cl | Cl | CH═C(Cl)COO—CH₂COOCH₃ | |
| 340 | Cl | Cl | CH═C(Cl)COO—CH(CH₃)COOCH₃ | |
| 341 | Cl | Cl | CH═C(Cl)CON(CH₃)₂ | |
| 342 | Cl | Cl | CH═C(Cl)CON(CH₃)—CH₂COOCH₃ | |
| 343 | Cl | Cl | CH═C(Cl)CONH—CH(CH₃)COOCH₃ | |
| 344 | Cl | Cl | CH═C(Br)COO—CH₃ | |
| 345 | Cl | Cl | CH═C(Br)COO—CH₂CH₃ | |
| 346 | Cl | Cl | CH═C(CH₃)COO—CH₃ | |
| 347 | Cl | Cl | CH═C(CH₃)COO—CH₂CH₃ | |
| 348 | Cl | Cl | CH₂—CH(Cl)—COO—CH₃ | |
| 349 | Cl | Cl | CH₂—CH(Cl)—COO—CH₂CH₃ | |
| 350 | Cl | Cl | CHO | |
| 351 | Cl | Cl | CH═N—OCH₃ | |
| 352 | Cl | Cl | CH═N—OCH₂CH₃ | |
| 353 | Cl | Cl | CH═N—OCH(CH₃)COOCH₃ | |
| 354 | Cl | Cl | SO₂Cl | |
| 355 | Cl | Cl | SO₂NH₂ | |
| 356 | Cl | Cl | SO₂NHCH₃ | |
| 357 | Cl | Cl | SO₂N(CH₃)₂ | |
| 358 | Cl | Cl | NH—CH₂C≡CH | |
| 359 | Cl | Cl | NHCH(CH₃)COOCH₃ | |
| 360 | Cl | Cl | N(CH₃)—CH₂C≡CH | |
| 361 | Cl | Cl | NH(SO₂CH₃) | |
| 362 | Cl | Cl | N(CH₃)(SO₂CH₃) | |
| 363 | Cl | Cl | N(SO₂CH₃)₂ | |
| 364 | Cl | CN | H | |
| 365 | Cl | CN | F | |
| 366 | Cl | CN | CH₃ | |
| 367 | Cl | CN | NO₂ | |
| 368 | Cl | CN | NH₂ | |
| 369 | Cl | CN | OH | |
| 370 | Cl | CN | OCH₃ | |
| 371 | Cl | CN | OCH(CH₃)₂ | |
| 372 | Cl | CN | O—CH₂CH═CH₂ | |
| 373 | Cl | CN | O—CH₂C≡CH | |
| 374 | Cl | CN | O—CH(CH₃)C≡CH | |
| 375 | Cl | CN | O-cyclopentyl | |
| 376 | Cl | CN | OCH₂COOH | |
| 377 | Cl | CN | OCH₂COO—CH₃ | |
| 378 | Cl | CN | OCH₂COO—CH₂CH₃ | |
| 379 | Cl | CN | OCH₂COO—CH₂CH═CH₂ | |
| 380 | Cl | CN | OCH₂COO—CH₂C≡CH | |
| 381 | Cl | CN | OCH₂COO—CH₂CH₂OCH₃ | |
| 382 | Cl | CN | OCH₂CONH—CH₃ | |
| 383 | Cl | CN | OCH₂CON(CH₃)₂ | |
| 384 | Cl | CN | OCH(CH₃)COOH | |
| 385 | Cl | CN | OCH(CH₃)COO—CH₃ | |
| 386 | Cl | CN | OCH(CH₃)COO—CH₂CH₃ | |
| 387 | Cl | CN | OCH(CH₃)COO—CH₂CH═CH₂ | |
| 388 | Cl | CN | OCH(CH₃)COO—CH₂C≡CH | |
| 389 | Cl | CN | OCH(CH₃)COO—CH₂CH₂OCH₃ | |
| 390 | Cl | CN | OCH(CH₃)CONH—CH₃ | |
| 391 | Cl | CN | OCH(CH₃)CON(CH₃)₂ | |
| 392 | Cl | CN | OC(CH₃)₂COO—CH₃ | |
| 393 | Cl | CN | OC(CH₃)₂COO—CH₂CH═CH₂ | |
| 394 | Cl | CN | SH | |
| 395 | Cl | CN | SCH₃ | |
| 396 | Cl | CN | SCH(CH₃)₂ | |
| 397 | Cl | CN | S—CH₂CH═CH₂ | |
| 398 | Cl | CN | S—CH₂C≡CH | |
| 399 | Cl | CN | S—CH(CH₃)C≡CH | |
| 400 | Cl | CN | S-cyclopentyl | |
| 401 | Cl | CN | SCH₂COOH | |
| 402 | Cl | CN | SCH₂COO—CH₃ | |
| 403 | Cl | CN | SCH₂COO—CH₂CH₃ | |
| 404 | Cl | CN | SCH₂COO—CH₂CH═CH₂ | |
| 405 | Cl | CN | SCH₂COO—CH₂C≡CH | |
| 406 | Cl | CN | SCH₂COO—CH₂CH₂OCH₃ | |
| 407 | Cl | CN | SCH₂CONH—CH₃ | |
| 408 | Cl | CN | SCH₂CON(CH₃)₂ | |
| 409 | Cl | CN | SCH(CH₃)COOH | |
| 410 | Cl | CN | SCH(CH₃)COO—CH₃ | |
| 411 | Cl | CN | SCH(CH₃)COO—CH₂CH₃ | |
| 412 | Cl | CN | SCH(CH₃)COO—CH₂CH═CH₂ | |
| 413 | Cl | CN | SCH(CH₃)COO—CH₂C≡CH | |
| 414 | Cl | CN | SCH(CH₃)COO—CH₂CH₂OCH₃ | |
| 415 | Cl | CN | SCH(CH₃)CONH—CH₃ | |
| 416 | Cl | CN | SCH(CH₃)CON(CH₃)₂ | |
| 417 | Cl | CN | SC(CH₃)₂COO—CH₃ | |
| 418 | Cl | CN | SC(CH₃)₂COO—CH₂CH═CH₂ | |
| 419 | Cl | CN | COOH | |
| 420 | Cl | CN | COOCH₃ | |
| 421 | Cl | CN | COOCH₂CH₃ | |
| 422 | Cl | CN | COOCH(CH₃)₂ | |
| 423 | Cl | CN | COO—CH₂CH═CH₂ | |
| 424 | Cl | CN | COO—CH₂C≡CH | |
| 425 | Cl | CN | COO-cyclopentyl | |
| 426 | Cl | CN | COO—CH₂COO—CH₃ | |
| 427 | Cl | CN | COO—CH₂COO—CH₂CH₃ | |
| 428 | Cl | CN | COO—CH₂COO—CH₂CH═CH₂ | |
| 429 | Cl | CN | COO—CH₂COO—CH₂C≡CH | |
| 430 | Cl | CN | COO—CH₂COO—CH₂CH₂OCH₃ | |
| 431 | Cl | CN | COO—CH(CH₃)COO—CH₃ | |
| 432 | Cl | CN | COO—CH(CH₃)COO—CH₂CH₃ | |
| 433 | Cl | CN | COO—CH(CH₃)COO—CH₂CH═CH₂ | |
| 434 | Cl | CN | COO—CH(CH₃)COO—CH₂C≡CH | |
| 435 | Cl | CN | COO—CH(CH₃)COO—CH₂CH₂OCH₃ | |
| 436 | Cl | CN | COO—C(CH₃)₂COO—CH₃ | |
| 437 | Cl | CN | COO—C(CH₃)₂COO—CH₂CH₃ | |
| 438 | Cl | CN | COO—C(CH₃)₂COO—CH₂CH═CH₂ | |
| 439 | Cl | CN | COO—C(CH₃)₂COO—CH₂C≡CH | |
| 440 | Cl | CN | COO—C(CH₃)₂COO—CH₂CH₂OCH₃ | |
| 441 | Cl | CN | CONH₂ | |
| 442 | Cl | CN | CONHCH₃ | |
| 443 | Cl | CN | CON(CH₃)₂ | |
| 444 | Cl | CN | CONH—CH₂COO—CH₃ | |
| 445 | Cl | CN | CONH—CH₂COO—CH₂CH═CH₂ | |
| 446 | Cl | CN | CONH—CH₂COO—CH₂CH₂OCH₃ | |
| 447 | Cl | CN | CONH—CH(CH₃)COO—CH₃ | |
| 448 | Cl | CN | CONH—CH(CH₃)COO—CH₂CH═CH₂ | |
| 449 | Cl | CN | CONH—CH(CH₃)COO—CH₂CH₂OCH₃ | |
| 450 | Cl | CN | CON(CH₃)—CH₂COO—CH₃ | |
| 451 | Cl | CN | CON(CH₃)—CH₂COO—CH₂CH═CH₂ | |
| 452 | Cl | CN | CON(CH₃)—CH₂COO—CH₂CH₂OCH₃ | |
| 453 | Cl | CN | C(═N—OCH₃)O—CH₃ | |
| 454 | Cl | CN | C(═N—OCH₃)O—CH₂—COOCH₃ | |
| 455 | Cl | CN | C(═N—OCH₃)O—CH₂—COO-phenyl | |
| 456 | Cl | CN | C(═N—OCH₃)O—CH(CH₃)—COOCH₃ | |
| 457 | Cl | CN | CH═C(Cl)COO—CH₃ | |
| 458 | Cl | CN | CH═C(Cl)COO—CH₂CH₃ | |
| 459 | Cl | CN | CH═C(Cl)COO—CH₂CH═CH₂ | |
| 460 | Cl | CN | CH═C(Cl)COO—CH₂COOCH₃ | |
| 461 | Cl | CN | CH═C(Cl)COO—CH(CH₃)COOCH₃ | |
| 462 | Cl | CN | CH═C(Cl)CON(CH₃)₂ | |
| 463 | Cl | CN | CH═C(Cl)CON(CH₃)—CH₂COOCH₃ | |
| 464 | Cl | CN | CH═C(Cl)CONH—CH(CH₃)COOCH₃ | |
| 465 | Cl | CN | CH═C(Br)COO—CH₃ | |
| 466 | Cl | CN | CH═C(Br)COO—CH₂CH₃ | |
| 467 | Cl | CN | CH═C(CH₃)COO—CH₃ | |
| 468 | Cl | CN | CH═C(CH₃)COO—CH₂CH₃ | |
| 469 | Cl | CN | CH₂—CH(Cl)—COO—CH₃ | |
| 470 | Cl | CN | CH₂—CH(Cl)—COO—CH₂CH₃ | |
| 471 | Cl | CN | CHO | |
| 472 | Cl | CN | CH═N—OCH₃ | |
| 473 | Cl | CN | CH═N—OCH₂CH₃ | |
| 474 | Cl | CN | CH═N—OCH(CH₃)COOCH₃ | |
| 475 | Cl | CN | SO₂Cl | |
| 476 | Cl | CN | SO₂NH₂ | |
| 477 | Cl | CN | SO₂NHCH₃ | |
| 478 | Cl | CN | SO₂N(CH₃)₂ | |
| 479 | Cl | CN | NH—CH₂C≡CH | |
| 480 | Cl | CN | NHCH(CH₃)COOCH₃ | |
| 481 | Cl | CN | N(CH₃)—CH₂C≡CH | |
| 482 | Cl | CN | NH(SO₂CH₃) | |
| 483 | Cl | CN | N(CH₃)(SO₂CH₃) | |
| 484 | Cl | CN | N(SO₂CH₃)₂ | |
| 485 | H | Cl | H | |
| 486 | H | Cl | F | |
| 487 | H | Cl | CH₃ | |
| 488 | H | Cl | NO₂ | |
| 489 | H | Cl | NH₂ | |
| 490 | H | Cl | OH | |
| 491 | H | Cl | OCH₃ | |
| 492 | H | Cl | OCH(CH₃)₂ | |
| 493 | H | Cl | O—CH₂CH═CH₂ | |
| 494 | H | Cl | O—CH₂C≡CH | |
| 495 | H | Cl | O—CH(CH₃)C≡CH | |
| 496 | H | Cl | O-cyclopentyl | |
| 497 | H | Cl | OCH₂COOH | |
| 498 | H | Cl | OCH₂COO—CH₃ | |
| 499 | H | Cl | OCH₂COO—CH₂CH₃ | |
| 500 | H | Cl | OCH₂COO—CH₂CH═CH₂ | |
| 501 | H | Cl | OCH₂COO—CH₂C≡CH | |
| 502 | H | Cl | OCH₂COO—CH₂CH₂OCH₃ | |
| 503 | H | Cl | OCH₂CONH—CH₃ | |
| 504 | H | Cl | OCH₂CON(CH₃)₂ | |
| 505 | H | Cl | OCH(CH₃)COOH | |
| 506 | H | Cl | OCH(CH₃)COO—CH₃ | |
| 507 | H | Cl | OCH(CH₃)COO—CH₂CH₃ | |
| 508 | H | Cl | OCH(CH₃)COO—CH₂CH═CH₂ | |
| 509 | H | Cl | OCH(CH₃)COO—CH₂C≡CH | |
| 510 | H | Cl | OCH(CH₃)COO—CH₂CH₂OCH₃ | |
| 511 | H | Cl | OCH(CH₃)CONH—CH₃ | |
| 512 | H | Cl | OCH(CH₃)CON(CH₃)₂ | |
| 513 | H | Cl | OC(CH₃)₂COO—CH₃ | |
| 514 | H | Cl | OC(CH₃)₂COO—CH₂CH═CH₂ | |
| 515 | H | Cl | SH | |
| 516 | H | Cl | SCH₃ | |
| 517 | H | Cl | SCH(CH₃)₂ | |
| 518 | H | Cl | S—CH₂CH═CH₂ | |
| 519 | H | Cl | S—CH₂C≡CH | |
| 520 | H | Cl | S—CH(CH₃)C≡CH | |
| 521 | H | Cl | S-cyclopentyl | |
| 522 | H | Cl | SCH₂COOH | |
| 523 | H | Cl | SCH₂COO—CH₃ | |
| 524 | H | Cl | SCH₂COO—CH₂CH₃ | |
| 525 | H | Cl | SCH₂COO—CH₂CH═CH₂ | |
| 526 | H | Cl | SCH₂COO—CH₂C≡CH | |
| 527 | H | Cl | SCH₂COO—CH₂CH₂OCH₃ | |
| 528 | H | Cl | SCH₂CONH—CH₃ | |
| 529 | H | Cl | SCH₂CON(CH₃)₂ | |
| 530 | H | Cl | SCH(CH₃)COOH | |
| 531 | H | Cl | SCH(CH₃)COO—CH₃ | |
| 532 | H | Cl | SCH(CH₃)COO—CH₂CH₃ | |
| 533 | H | Cl | SCH(CH₃)COO—CH₂CH═CH₂ | |
| 534 | H | Cl | SCH(CH₃)COO—CH₂C≡CH | |
| 535 | H | Cl | SCH(CH₃)COO—CH₂CH₂OCH₃ | |
| 536 | H | Cl | SCH(CH₃)CONH—CH₃ | |
| 537 | H | Cl | SCH(CH₃)CON(CH₃)₂ | |
| 538 | H | Cl | SC(CH₃)₂COO—CH₃ | |
| 539 | H | Cl | SC(CH₃)₂COO—CH₂CH═CH₂ | |
| 540 | H | Cl | COOH | |
| 541 | H | Cl | COOCH₃ | |
| 542 | H | Cl | COOCH₂CH₃ | |
| 543 | H | Cl | COOCH(CH₃)₂ | |
| 544 | H | Cl | COO—CH₂CH═CH₂ | |
| 545 | H | Cl | COO—CH₂C≡CH | |
| 546 | H | Cl | COO-cyclopentyl | |
| 547 | H | Cl | COO—CH₂COO—CH₃ | |
| 548 | H | Cl | COO—CH₂COO—CH₂CH₃ | |
| 549 | H | Cl | COO—CH₂COO—CH₂CH═CH₂ | |
| 550 | H | Cl | COO—CH₂COO—CH₂C≡CH | |
| 551 | H | Cl | COO—CH₂COO—CH₂CH₂OCH₃ | |
| 552 | H | Cl | COO—CH(CH₃)COO—CH₃ | |
| 553 | H | Cl | COO—CH(CH₃)COO—CH₂CH₃ | |
| 554 | H | Cl | COO—CH(CH₃)COO—CH₂CH═CH₂ | |
| 555 | H | Cl | COO—CH(CH₃)COO—CH₂C≡CH | |
| 556 | H | Cl | COO—CH(CH₃)COO—CH₂CH₂OCH₃ | |
| 557 | H | Cl | COO—C(CH₃)₂COO—CH₃ | |
| 558 | H | Cl | COO—C(CH₃)₂COO—CH₂CH₃ | |
| 559 | H | Cl | COO—C(CH₃)₂COO—CH₂CH═CH₂ | |
| 560 | H | Cl | COO—C(CH₃)₂COO—CH₂C≡CH | |
| 561 | H | Cl | COO—C(CH₃)₂COO—CH₂CH₂OCH₃ | |
| 562 | H | Cl | CONH₂ | |
| 563 | H | Cl | CONHCH₃ | |
| 564 | H | Cl | CON(CH₃)₂ | |
| 565 | H | Cl | CONH—CH₂COO—CH₃ | |
| 566 | H | Cl | CONH—CH₂COO—CH₂CH═CH₂ | |
| 567 | H | Cl | CONH—CH₂COO—CH₂CH₂OCH₃ | |
| 568 | H | Cl | CONH—CH(CH₃)COO—CH₃ | |
| 569 | H | Cl | CONH—CH(CH₃)COO—CH₂CH═CH₂ | |
| 570 | H | Cl | CONH—CH(CH₃)COO—CH₂CH₂OCH₃ | |
| 571 | H | Cl | CON(CH₃)—CH₂COO—CH₃ | |
| 572 | H | Cl | CON(CH₃)—CH₂COO—CH₂CH═CH₂ | |
| 573 | H | Cl | CON(CH₃)—CH₂COO—CH₂CH₂OCH₃ | |
| 574 | H | Cl | C(═N—OCH₃)O—CH₃ | |
| 575 | H | Cl | C(═N—OCH₃)O—CH₂—COOCH₃ | |
| 576 | H | Cl | C(═N—OCH₃)O—CH₂—COO-phenyl | |
| 577 | H | Cl | C(═N—OCH₃)O—CH(CH₃)—COOCH₃ | |
| 578 | H | Cl | CH═C(Cl)COO—CH₃ | |
| 579 | H | Cl | CH═C(Cl)COO—CH₂CH₃ | |
| 580 | H | Cl | CH═C(Cl)COO—CH₂CH═CH₂ | |
| 581 | H | Cl | CH═C(Cl)COO—CH₂COOCH₃ | |
| 582 | H | Cl | CH═C(Cl)COO—CH(CH₃)COOCH₃ | |
| 583 | H | Cl | CH═C(Cl)CON(CH₃)₂ | |
| 584 | H | Cl | CH═C(Cl)CON(CH₃)—CH₂COOCH₃ | |
| 585 | H | Cl | CH═C(Cl)CONH—CH(CH₃)COOCH₃ | |
| 586 | H | Cl | CH═C(Br)COO—CH₃ | |
| 587 | H | Cl | CH═C(Br)COO—CH₂CH₃ | |
| 588 | H | Cl | CH═C(CH₃)COO—CH₃ | |
| 589 | H | Cl | CH═C(CH₃)COO—CH₂CH₃ | |
| 590 | H | Cl | CH₂—CH(Cl)—COO—CH₃ | |
| 591 | H | Cl | CH₂—CH(Cl)—COO—CH₂CH₃ | |
| 592 | H | Cl | CHO | |
| 593 | H | Cl | CH═N—OCH₃ | |
| 594 | H | Cl | CH═N—OCH₂CH₃ | |
| 595 | H | Cl | CH═N—OCH(CH₃)COOCH₃ | |
| 596 | H | Cl | SO₂Cl | |
| 597 | H | Cl | SO₂NH₂ | |
| 598 | H | Cl | SO₂NHCH₃ | |
| 599 | H | Cl | SO₂N(CH₃)₂ | |
| 600 | H | Cl | NH—CH₂C≡CH | |
| 601 | H | Cl | NHCH(CH₃)COOCH₃ | |
| 602 | H | Cl | N(CH₃)—CH₂C≡CH | |
| 603 | H | Cl | NH(SO₂CH₃) | |
| 604 | H | Cl | N(CH₃)(SO₂CH₃) | |
| 605 | H | Cl | N(SO₂CH₃)₂ | |
| 606 | H | CN | N | |
| 607 | H | CN | F | |
| 608 | H | CN | CH₃ | |
| 609 | H | CN | NO₂ | |
| 610 | H | CN | NH₂ | |
| 611 | H | CN | OH | |
| 612 | H | CN | OCH₃ | |
| 613 | H | CN | OCH(CH₃)₂ | |
| 614 | H | CN | O—CH₂CH═CH₂ | |
| 615 | H | CN | O—CH₂C≡CH | |
| 616 | H | CN | O—CH(CH₃)C≡CH | |
| 617 | H | CN | O-cyclopentyl | |
| 618 | H | CN | OCH₂COOH | |
| 619 | H | CN | OCH₂COO—CH₃ | |
| 620 | H | CN | OCH₂COO—CH₂CH₃ | |
| 621 | H | CN | OCH₂COO—CH₂CH═CH₂ | |
| 622 | H | CN | OCH₂COO—CH₂C≡CH | |
| 623 | H | CN | OCH₂COO—CH₂CH₂OCH₃ | |
| 624 | H | CN | OCH₂CONH—CH₃ | |
| 625 | H | CN | OCH₂CON(CH₃)₂ | |
| 626 | H | CN | OCH(CH₃)COOH | |
| 627 | H | CN | OCH(CH₃)COO—CH₃ | |
| 628 | H | CN | OCH(CH₃)COO—CH₂CH₃ | |
| 629 | H | CN | OCH(CH₃)COO—CH₂CH═CH₂ | |
| 630 | H | CN | OCH(CH₃)COO—CH₂C≡CH | |
| 631 | H | CN | OCH(CH₃)COO—CH₂CH₂OCH₃ | |
| 632 | H | CN | OCH(CH₃)CONH—CH₃ | |
| 633 | H | CN | OCH(CH₃)CON(CH₃)₂ | |
| 634 | H | CN | OC(CH₃)₂COO—CH₃ | |
| 635 | H | CN | OC(CH₃)₂COO—CH₂CH═CH₂ | |
| 636 | H | CN | SH | |
| 637 | H | CN | SCH₃ | |
| 638 | H | CN | SCH(CH₃)₂ | |
| 639 | H | CN | S—CH₂CH═CH₂ | |
| 640 | H | CN | S—CH₂C≡CH | |
| 641 | H | CN | S—CH(CH₃)C≡CH | |
| 642 | H | CN | S-cyclopentyl | |
| 643 | H | CN | SCH₂COOH | |
| 644 | H | CN | SCH₂COO—CH₃ | |
| 645 | H | CN | SCH₂COO—CH₂CH₃ | |
| 646 | H | CN | SCH₂COO—CH₂CH═CH₂ | |
| 647 | H | CN | SCH₂COO—CH₂C≡CH | |
| 648 | H | CN | SCH₂COO—CH₂CH₂OCH₃ | |
| 649 | H | CN | SCH₂CONH—CH₃ | |
| 650 | H | CN | SCH₂CON(CH₃)₂ | |
| 651 | H | CN | SCH(CH₃)COOH | |
| 652 | H | CN | SCH(CH₃)COO—CH₃ | |
| 653 | H | CN | SCH(CH₃)COO—CH₂CH₃ | |
| 654 | H | CN | SCH(CH₃)COO—CH₂CH═CH₂ | |
| 655 | H | CN | SCH(CH₃)COO—CH₂C≡CH | |
| 656 | H | CN | SCH(CH₃)COO—CH₂CH₂OCH₃ | |
| 657 | H | CN | SCH(CH₃)CONH—CH₃ | |
| 658 | H | CN | SCH(CH₃)CON(CH₃)₂ | |
| 659 | H | CN | SC(CH₃)₂COO—CH₃ | |
| 660 | H | CN | SC(CH₃)₂COO—CH₂CH═CH₂ | |
| 661 | H | CN | COOH | |
| 662 | H | CN | COOCH₃ | |
| 663 | H | CN | COOCH₂CH₃ | |
| 664 | H | CN | COOCH(CH₃)₂ | |
| 665 | H | CN | COO—CH₂CH═CH₂ | |
| 666 | H | CN | COO—CH₂C≡CH | |
| 667 | H | CN | COO-cyclopentyl | |
| 668 | H | CN | COO—CH₂COO—CH₃ | |
| 669 | H | CN | COO—CH₂COO—CH₂CH₃ | |
| 670 | H | CN | COO—CH₂COO—CH₂CH═CH₂ | |
| 671 | H | CN | COO—CH₂COO—CH₂C≡CH | |
| 672 | H | CN | COO—CH₂COO—CH₂CH₂OCH₃ | |
| 673 | H | CN | COO—CH(CH₃)COO—CH₃ | |
| 674 | H | CN | COO—CH(CH₃)COO—CH₂CH₃ | |
| 675 | H | CN | COO—CH(CH₃)COO—CH₂CH═CH₂ | |
| 676 | H | CN | COO—CH(CH₃)COO—CH₂C≡CH | |
| 677 | H | CN | COO—CH(CH₃)COO—CH₂CH₂OCH₃ | |
| 678 | H | CN | COO—C(CH₃)₂COO—CH₃ | |
| 679 | H | CN | COO—C(CH₃)₂COO—CH₂CH₃ | |
| 680 | H | CN | COO—C(CH₃)₂COO—CH₂CH═CH₂ | |
| 681 | H | CN | COO—C(CH₃)₂COO—CH₂C≡CH | |
| 682 | H | CN | COO—C(CH₃)₂COO—CH₂CH₂OCH₃ | |
| 683 | H | CN | CONH₂ | |
| 684 | H | CN | CONHCH₃ | |
| 685 | H | CN | CON(CH₃)₂ | |
| 686 | H | CN | CONH—CH₂COO—CH₃ | |
| 687 | H | CN | CONH—CH₂COO—CH₂CH═CH₂ | |
| 688 | H | CN | CONH—CH₂COO—CH₂CH₂OCH₃ | |
| 689 | H | CN | CONH—CH(CH₃)COO—CH₃ | |
| 690 | H | CN | CONH—CH(CH₃)COO—CH₂CH═CH₂ | |
| 691 | H | CN | CONH—CH(CH₃)COO—CH₂CH₂OCH₃ | |
| 692 | H | CN | CON(CH₃)—CH₂COO—CH₃ | |
| 693 | H | CN | CON(CH₃)—CH₂COO—CH₂CH═CH₂ | |
| 694 | H | CN | CON(CH₃)—CH₂COO—CH₂CH₂OCH₃ | |
| 695 | H | CN | C(═N—OCH₃O)O—CH₃ | |
| 696 | H | CN | C(═N—OCH₃)O—CH₂—COOCH₃ | |
| 697 | H | CN | C(═N—OCH₃)O—CH₂—COO-phenyl | |
| 698 | H | CN | C(═N—OCH₃)O—CH(CH₃)—COOCH₃ | |
| 699 | H | CN | CH═C(Cl)COO—CH₃ | |
| 700 | H | CN | CH═C(Cl)COO—CH₂CH₃ | |
| 701 | H | CN | CH═C(Cl)COO—CH₂CH═CH₂ | |
| 702 | H | CN | CH═C(Cl)COO—CH₂COOCH₃ | |
| 703 | H | CN | CH═C(Cl)COO—CH(CH₃)COOCH₃ | |
| 704 | H | CN | CH═C(Cl)CON(CH₃)₂ | |
| 705 | H | CN | CH═C(Cl)CON(CH₃)—CH₂COOCH₃ | |
| 706 | H | CN | CH═C(Cl)CONH—CH(CH₃)COOCH₃ | |
| 707 | H | CN | CH═C(Br)COO—CH₃ | |
| 708 | H | CN | CH═C(Br)COO—CH₂CH₃ | |
| 709 | H | CN | CH═C(CH₃)COO—CH₃ | |
| 710 | H | CN | CH═C(CH₃)COO—CH₂CH₃ | |
| 711 | H | CN | CH₂—CH(Cl)—COO—CH₃ | |
| 712 | H | CN | CH₂—CH(Cl)—COO—CH₂CH₃ | |
| 713 | H | CN | CHO | |
| 714 | H | CN | CH═N—OCH₃ | |
| 715 | H | CN | CH═N—OCH₂CH₃ | |
| 716 | H | CN | CH═N—OCH(CH₃)COOCH₃ | |
| 717 | H | CN | SO₂Cl | |
| 718 | H | CN | SO₂NH₂ | |
| 719 | H | CN | SO₂NHCH₃ | |
| 720 | H | CN | SO₂N(CH₃)₂ | |
| 721 | H | CN | NH—CH₂C≡CH | |
| 722 | H | CN | NHCH(CH₃)COOCH₃ | |
| 723 | H | CN | N(CH₃)—CH₂C≡CH | |
| 724 | H | CN | NH(SO₂CH₃) | |
| 725 | H | CN | N(CH₃)(SO₂CH₃) | |
| 726 | H | CN | N(SO₂CH₃)₂ | |
| 727 | F | Cl | OCH(CH₃)COO—CH₃ | (R enantiomer) |
| 728 | F | Cl | OCH(CH₃)COO—CH₂CH₃ | (R enantiomer) |
| 729 | F | Cl | OCH(CH₃)COO—CH₂CH═CH₂ | (R enantiomer) |
| 730 | F | Cl | OCH(CH₃)COO—CH₂C≡CH | (R enantiomer) |
| 731 | F | Cl | OCH(CH₃)COO—CH₂CH₂OCH₃ | (R enantiomer) |
| 732 | F | Cl | OCH(CH₃)CONH—CH₃ | (R enantiomer) |
| 733 | F | Cl | OCH(CH₃)CON(CH₃)₂ | (R enantiomer) |
| 734 | F | CN | OCH(CH₃)COO—CH₃ | (R enantiomer) |
| 735 | F | CN | OCH(CH₃)COO—CH₂CH₃ | (R enantiomer) |
| 736 | F | CN | OCH(CH₃)COO—CH₂CH═CH₂ | (R enantiomer) |
| 737 | F | CN | OCH(CH₃)COO—CH₂C≡CH | (R enantiomer) |
| 738 | F | CN | OCH(CH₃)COO—CH₂CH₂OCH₃ | (R enantiomer) |
| 739 | F | CN | OCH(CH₃)CONH—CH₃ | (R enantiomer) |
| 740 | F | CN | OCH(CH₃)CON(CH₃)₂ | (R enantiomer) |
| 741 | H | Cl | OCH(CH₃)COO—CH₃ | (R enantiomer) |
| 742 | H | Cl | OCH(CH₃)COO—CH₂CH₃ | (R enantiomer) |
| 743 | H | Cl | OCH(CH₃)COO—CH₂CH═CH₂ | (R enantiomer) |
| 744 | H | Cl | OCH(CH₃)COO—CH₂C≡CH | (R enantiomer) |
| 745 | H | Cl | OCH(CH₃)COO—CH₂CH₂OCH₃ | (R enantiomer) |
| 746 | H | Cl | OCH(CH₃)CONH—CH₃ | (R enantiomer) |
| 747 | H | Cl | OCH(CH₃)CON(CH₃)₂ | (R enantiomer) |
| 748 | H | CN | OCH(CH₃)COO—CH₃ | (R enantiomer) |
| 749 | H | CN | OCH(CH₃)COO—CH₂CH₃ | (R enantiomer) |
| 750 | H | CN | OCH(CH₃)COO—CH₂CH═CH₂ | (R enantiomer) |
| 751 | H | CN | OCH(CH₃)COO—CH₂C≡CH | (R enantiomer) |
| 752 | H | CN | OCH(CH₃)COO—CH₂CH₂OCH₃ | (R enantiomer) |
| 753 | H | CN | OCH(CH₃)CONH—CH₃ | (R enantiomer) |
| 754 | H | CN | OCH(CH₃)CON(CH₃)₂ | (R enantiomer) |
| 755 | Cl | Cl | OCH(CH₃)COO—CH₃ | (R enantiomer) |
| 756 | Cl | Cl | OCH(CH₃)COO—CH₂CH₃ | (R enantiomer) |
| 757 | Cl | Cl | OCH(CH₃)COO—CH₂CH═CH | (R enantiomer) |
| 758 | Cl | Cl | OCH(CH₃)COO—CH₂C≡CH | (R enantiomer) |
| 759 | ci | Cl | OCH(CH₃)COO—CH₂CH₂OCH₃ | (R enantiomer) |
| 760 | Cl | Cl | OCH(CH₃)CONH—CH₃ | (R enantiomer) |
| 761 | Cl | Cl | OCH(CH₃)CON(CH₃)₂ | (R enantiomer) |
| 762 | Cl | CN | OCH(CH₃)COO—CH₃ | (R enantiomer) |
| 763 | Cl | CN | OCH(CH₃)COO—CH₂CH₃ | (R enantiomer) |
| 764 | Cl | CN | OCH(CH₃)COO—CH₂CH═CH₂ | (R enantiomer) |
| 765 | Cl | CN | OCH(CH₃)COO—CH₂C≡CH | (R enantiomer) |
| 766 | Cl | CN | OCH(CH₃)COO—CH₂CH₂OCH₃ | (R enantiomer) |
| 767 | Cl | CN | OCH(CH₃)CONH—CH₃ | (R enantiomer) |
| 768 | Cl | CN | OCH(CH₃)CON(CH₃)₂ | (R enantiomer) |
| 769 | Cl | Cl | N(SO₂C₂H₅)₂ | |
| 770 | Cl | Cl | NH(SO₂C₂H₅) | |
| 771 | Cl | CN | N(SO₂C₂H₅)₂ | |
| 772 | Cl | CN | NH(SO₂C₂H₅) | |
| 773 | H | Cl | N(SO₂C₂H₅)₂ | |
| 774 | H | Cl | NH(SO₂C₂H₅) | |
| 775 | H | CN | N(SO₂C₂H₅)₂ | |
| 776 | Cl | CN | NH(SO₂C₂H₅) | |

Particular preference is also given to the compounds of the formula IAb (compounds IA where Q=CH, R¹=CF₃ and R²=Br) in which the variables R³, R⁴ and X—R⁵ together have the meanings given in each case in one row of Table I (compounds IAb.1-IAb.776).

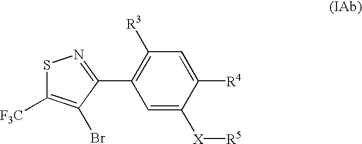

Particular preference is given to the compounds of the formula IAc (compounds IA where Q=CH, R¹=OCHF₂ and R²=Cl) in which the variables R³, R⁴ and X—R⁵ together have the meanings given in each case in one row of Table 1 (compounds IAc.1-IAc.776).

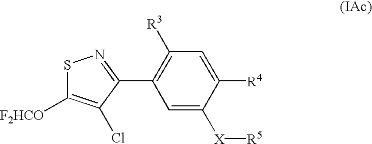

Particular preference is given to the compounds of the formula IAd (compounds IA where Q=CH, R¹=OCHF₂ and R²=Br) in which the variables R³, R⁴ and X—R⁵ together have the meanings given in each case in one row of Table 1 (compounds IAd.1-IAd.776).

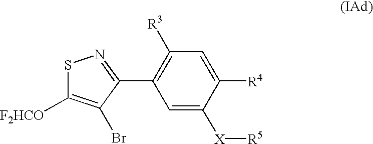

Particular preference is given to the compounds of the formula IAe (compounds IA where Q=CH, R¹=SO₂CH₃ and R²=Cl) in which the variables R³, R⁴ and X—R⁵ together have the meanings given in each case in one row of Table 1 (compounds IAe.1-IAe.776).

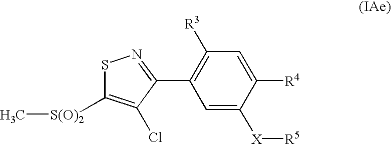

Particular preference is given to the compounds of the formula IAf (compounds IA where Q=CH, R¹=OSO₂CH₃ and R²=Cl) in which the variables R³, R⁴ and X—R⁵ together have the meanings given in each case in one row of Table 1 (compounds IAf.1-IAf.776).

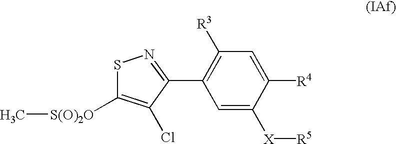

Preference is furthermore given to the compounds of the formula IBa (compounds IB where Q=N, R¹=CF₃ and R²=Cl) in which the variables R³, R⁴ and X—R⁵ together have the meanings given in each case in one row of Table 1 (compounds IBa.1-IBa.776).

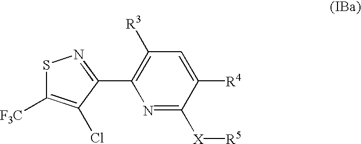

Preference is furthermore also given to the compounds of the formula IBb (compounds IB where Q=N, R¹=CF₃ and R²=Br) in which the variables R³, R⁴ and X—R⁵ together have the meanings given in each case in one row of Table 1 (compounds IBb.1-IBb.776).

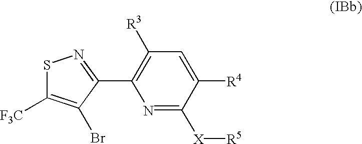

Preference is also given to the compounds of the formula IBc (compounds IB where Q=N, R¹=OCHF₂ and R²=Cl) in which the variables R³, R⁴ and X—R⁵ together have the meanings given in each case in one row of Table 1 (compounds IBc.1-IBc.776).

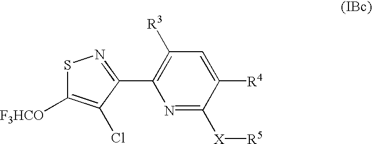

Preference is also given to the compounds of the formula IBd (compounds IB where Q=N, R¹=OCHF₂ and R²=Br) in which the variables R³, R⁴ and X—R⁵ together have the meanings given in each case in one row of Table 1 (compounds IBd.1-IBd.776).

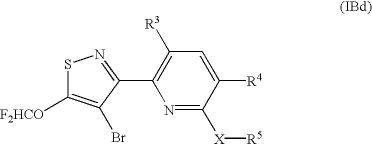

Preference is also given to the compounds of the formula IBe (compounds IB where Q=N, R¹=SO₂CH₃ and R²=Cl) in which the variables R³, R⁴ and X—R⁵ together have the meanings given in each case in one row of Table 1 (compounds IBe.1-IBe.776).

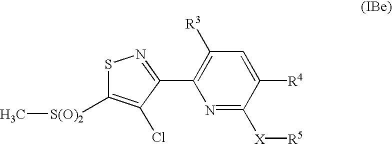

Preference is also given to the compounds of the formula IBf (compounds IB where Q=N, R¹=OSO₂CH₃ and R²=Cl) in which the variables R³, R⁴ and X—R⁵ together have the meanings given in each case in one row of Table 1 (compounds IBf.1-IBf.776).

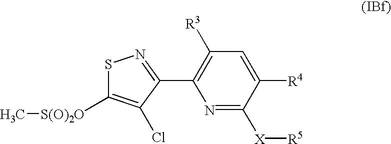

Examples of preferred compounds IC are the compounds of the formula ICa (compounds IC where Q=CH, R¹=CF₃, R²=Cl in which R⁴ and X—R⁵ form a chain —OCH(R¹⁵)—C(O)—NR¹⁷—) in which the variables R³, R¹⁵ and R¹⁷ together have the meanings given in each case in one row of Table 2 (compounds ICa.1-ICa.204).

TABLE 2

| | |
|---|---|
| | (ICa) |
| 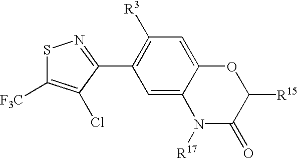 | |

| No. | R³ | R¹⁵ | R¹⁷ |
|---|---|---|---|
| 1 | F | H | H |
| 2 | F | H | CH₃ |
| 3 | F | H | C₂H₅ |
| 4 | F | H | n-C₃H₇ |
| 5 | F | H | CH(CH₃)₂ |
| 6 | F | H | n-C₄H₉ |
| 7 | F | H | CH(CH₃)—C₂H₅ |
| 8 | F | H | CH₂—CH(CH₃)₂ |
| 9 | F | H | CH₂—CF₃ |
| 10 | F | H | CH₂—CH═CH₂ |
| 11 | F | H | CH₂—C≡CH |
| 12 | F | H | CH(CH₃)—C≡CH |
| 13 | F | H | CH₂—COOCH₃ |
| 14 | F | H | CH₂—COOC₂H₅ |
| 15 | F | H | CH(CH₃)—COOCH₃ |
| 16 | F | H | CH(CH₃)—COOC₂H₅ |
| 17 | F | H | OH |
| 18 | F | H | OCH₃ |
| 19 | F | H | OC₂H₅ |
| 20 | F | H | O-n-C₃H₇ |
| 21 | F | H | OCH(CH₃)₂ |
| 22 | F | H | O-n-C₄H₉ |
| 23 | F | H | OCH(CH₃)—C₂H₅ |
| 24 | F | H | OCH₂—CH(CH₃)₂ |
| 25 | F | H | OCH₂—CH═CH₂ |
| 26 | F | H | OCH₂—C≡CH |
| 27 | F | H | OCH(CH₃)—C≡CH |
| 28 | F | H | OCH₂—COOCH₃ |
| 29 | F | H | OCH₂—COOC₂H₅ |
| 30 | F | H | OCH(CH₃)—COOCH₃ |
| 31 | F | H | OCH(CH₃)—COOC₂H₅ |
| 32 | F | H | OCH₂—CF₃ |
| 33 | F | H | Ocyclopropyl |
| 34 | F | H | OCH₂-cyclopropyl |
| 35 | F | CH₃ | H |
| 36 | F | CH₃ | CH₃ |
| 37 | F | CH₃ | C₂H₅ |
| 38 | F | CH₃ | n-C₃H₇ |
| 39 | F | CH₃ | CH(CH₃)₂ |
| 40 | F | CH₃ | n-C₄H₉ |
| 41 | F | CH₃ | CH(CH₃)—C₂H₅ |
| 42 | F | CH₃ | CH₂—CH(CH₃)₂ |
| 43 | F | CH₃ | CH₂—CF₃ |
| 44 | F | CH₃ | CH₂—CH═CH₂ |
| 45 | F | CH₃ | CH₂—C≡CH |
| 46 | F | CH₃ | CH(CH₃)—C≡CH |
| 47 | F | CH₃ | CH₂—COOCH₃ |
| 48 | F | CH₃ | CH₂—COOC₂H₅ |
| 49 | F | CH₃ | CH(CH₃)—COOCH₃ |
| 50 | F | CH₃ | CH(CH₃)—COOC₂H₅ |
| 51 | F | CH₃ | OH |
| 52 | F | CH₃ | OCH₃ |
| 53 | F | CH₃ | OC₂H₅ |
| 54 | F | CH₃ | O-n-C₃H₇ |
| 55 | F | CH₃ | OCH(CH₃)₂ |
| 56 | F | CH₃ | O-n-C₄H₉ |
| 57 | F | CH₃ | OCH(CH₃)—C₂H₅ |
| 58 | F | CH₃ | OCH₂—CH(CH₃)₂ |
| 59 | F | CH₃ | OCH₂—CH═CH₂ |
| 60 | F | CH₃ | OCH₂—C≡CH |
| 61 | F | CH₃ | OCH(CH₃)—C≡CH |
| 62 | F | CH₃ | OCH₂—COOCH₃ |
| 63 | F | CH₃ | OCH₂—COOC₂H₅ |
| 64 | F | CH₃ | OCH(CH₃)—COOCH₃ |
| 65 | F | CH₃ | OCH(CH₃)—COOC₂H₅ |
| 66 | F | CH₃ | OCH₂—CF₃ |
| 67 | F | CH₃ | Ocyclopropyl |
| 68 | F | CH₃ | OCH₂-cyclopropyl |
| 69 | Cl | H | H |
| 70 | Cl | H | CH₃ |
| 71 | Cl | H | C₂H₅ |
| 72 | Cl | H | n-C₃H₇ |
| 73 | Cl | H | CH(CH₃)₂ |
| 74 | Cl | H | n-C₄H₉ |
| 75 | Cl | H | CH(CH₃)—C₂H₅ |
| 76 | Cl | H | CH₂—CH(CH₃)₂ |
| 77 | Cl | H | CH₂—CF₃ |
| 78 | Cl | H | CH₂—CH═CH₂ |
| 79 | Cl | H | CH₂—C≡CH |
| 80 | Cl | H | CH(CH₃)—C≡CH |
| 81 | Cl | H | CH₂—COOCH₃ |
| 82 | Cl | H | CH₂—COOC₂H₅ |
| 83 | Cl | H | CH(CH₃)—COOCH₃ |
| 84 | Cl | H | CH(CH₃)—COOC₂H₅ |
| 85 | Cl | H | OH |
| 86 | Cl | H | OCH₃ |
| 87 | Cl | H | OC₂H₅ |
| 88 | Cl | H | O-n-C₃H₇ |
| 89 | Cl | H | OCH(CH₃)₂ |
| 90 | Cl | H | O-n-C₄H₉ |
| 91 | Cl | H | OCH(CH₃)—C₂H₅ |
| 92 | Cl | H | OCH₂—CH(CH₃)₂ |
| 93 | Cl | H | OCH₂—CH═CH₂ |
| 94 | Cl | H | OCH₂—C≡CH |
| 95 | Cl | H | OCH(CH₃)—C≡CH |
| 96 | Cl | H | OCH₂—COOCH₃ |
| 97 | Cl | H | OCH₂—COOC₂H₅ |
| 98 | Cl | H | OCH(CH₃)—COOCH₃ |
| 99 | Cl | H | OCH(CH₃)—COOC₂H₅ |
| 100 | Cl | H | OCH₂—CF₃ |
| 101 | Cl | H | Ocyclopropyl |
| 102 | Cl | H | OCH₂-cyclopropyl |
| 103 | Cl | CH₃ | H |
| 104 | Cl | CH₃ | CH₃ |
| 105 | Cl | CH₃ | C₂H₅ |
| 106 | Cl | CH₃ | n-C₃H₇ |
| 107 | Cl | CH₃ | CH(CH₃)₂ |
| 108 | Cl | CH₃ | n-C₄H₉ |
| 109 | Cl | CH₃ | CH(CH₃)—C₂H₅ |
| 110 | Cl | CH₃ | CH₂—CH(CH₃)₂ |
| 111 | Cl | CH₃ | CH₂—CF₃ |
| 112 | Cl | CH₃ | CH₂—CH═CH₂ |
| 113 | Cl | CH₃ | CH₂—C≡CH |
| 114 | Cl | CH₃ | CH(CH₃)—C≡CH |
| 115 | Cl | CH₃ | CH₂—COOCH₃ |
| 116 | Cl | CH₃ | CH₂—COOC₂H₅ |
| 117 | Cl | CH₃ | CH(CH₃)—COOCH₃ |
| 118 | Cl | CH₃ | CH(CH₃)—COOC₂H₅ |
| 119 | Cl | CH₃ | OH |
| 120 | Cl | CH₃ | OCH₃ |
| 121 | Cl | CH₃ | OC₂H₅ |
| 122 | Cl | CH₃ | O-n-C₃H₇ |
| 123 | Cl | CH₃ | OCH(CH₃)₂ |
| 124 | Cl | CH₃ | O-n-C₄H₉ |
| 125 | Cl | CH₃ | OCH(CH₃)—C₂H₅ |
| 126 | Cl | CH₃ | OCH₂—CH(CH₃)₂ |
| 127 | Cl | CH₃ | OCH₂—CH═CH₂ |
| 128 | Cl | CH₃ | OCH₂—C≡CH |
| 129 | Cl | CH₃ | OCH(CH₃)—C≡CH |
| 130 | Cl | CH₃ | OCH₂—COOCH₃ |
| 131 | Cl | CH₃ | OCH₂—COOC₂H₅ |
| 132 | Cl | CH₃ | OCH(CH₃)—COOCH₃ |
| 133 | Cl | CH₃ | OCH(CH₃)—COOC₂H₅ |
| 134 | Cl | CH₃ | OCH₂—CF₃ |
| 135 | Cl | CH₃ | Ocyclopropyl |
| 136 | Cl | CH₃ | OCH₂-cyclopropyl |
| 137 | H | H | H |
| 138 | H | H | CH₃ |
| 139 | H | H | C₂H₅ |
| 140 | H | H | n-C₃H₇ |
| 141 | H | H | CH(CH₃)₂ |
| 142 | H | H | n-C₄H₉ |
| 143 | H | H | CH(CH₃)—C₂H₅ |
| 144 | H | H | CH₂—CH(CH₃)₂ |
| 145 | H | H | CH₂—CF₃ |
| 146 | H | H | CH₂—CH═CH₂ |
| 147 | H | H | CH₂—C≡CH |
| 148 | H | H | CH(CH₃)—C≡CH |
| 149 | H | H | CH₂—COOCH₃ |
| 150 | H | H | CH₂—COOC₂H₅ |
| 151 | H | H | CH(CH₃)—COOCH₃ |
| 152 | H | H | CH(CH₃)—COOC₂H₅ |
| 153 | H | H | OH |
| 154 | H | H | OCH₃ |
| 155 | H | H | OC₂H₅ |
| 156 | H | H | O-n-C₃H₇ |
| 157 | H | H | OCH(CH₃)₂ |
| 158 | H | H | O-n-C₄H₉ |
| 159 | H | H | OCH(CH₃)—C₂H₅ |
| 160 | H | H | OCH₂—CH(CH₃)₂ |
| 161 | H | H | OCH₂—CH═CH₂ |
| 162 | H | H | OCH₂—C≡CH |
| 163 | H | H | OCH(CH₃)—C≡CH |
| 164 | H | H | OCH₂—COOCH₃ |
| 165 | H | H | OCH₂—COOC₂H₅ |
| 166 | H | H | OCH(CH₃)—COOCH₃ |
| 167 | H | H | OCH(CH₃)—COOC₂H₅ |
| 168 | H | H | OCH₂—CF₃ |
| 169 | H | H | Ocyclopropyl |
| 170 | H | H | OCH₂-cyclopropyl |
| 171 | H | CH₃ | H |
| 172 | H | CH₃ | CH₃ |
| 173 | H | CH₃ | C₂H₅ |
| 174 | H | CH₃ | n-C₃H₇ |
| 175 | H | CH₃ | CH(CH₃)₂ |
| 176 | H | CH₃ | n-C₄H₉ |
| 177 | H | CH₃ | CH(CH₃)—C₂H₅ |
| 178 | H | CH₃ | CH₂—CH(CH₃)₂ |
| 179 | H | CH₃ | CH₂—CF₃ |
| 180 | H | CH₃ | CH₂—CH═CH₂ |
| 181 | H | CH₃ | CH₂—C≡CH |
| 182 | H | CH₃ | CH(CH₃)—C≡CH |
| 183 | H | CH₃ | CH₂—COOCH₃ |
| 184 | H | CH₃ | CH₂—COOC₂H₅ |
| 185 | H | CH₃ | CH(CH₃)—COOCH₃ |
| 186 | H | CH₃ | CH(CH₃)—COOC₂H₅ |
| 187 | H | CH₃ | OH |
| 188 | H | CH₃ | OCH₃ |
| 189 | H | CH₃ | OC₂H₅ |
| 190 | H | CH₃ | O-n-C₃H₇ |
| 191 | H | CH₃ | OCH(CH₃)₂ |
| 192 | H | CH₃ | O-n-C₄H₉ |
| 193 | H | CH₃ | OCH(CH₃)—C₂H₅ |
| 194 | H | CH₃ | OCH₂—CH(CH₃)₂ |
| 195 | H | CH₃ | OCH₂—CH═CH₂ |
| 196 | H | CH₃ | OCH₂—C≡CH |
| 197 | H | CH₃ | OCH(CH₃)—C≡CH |
| 198 | H | CH₃ | OCH₂—COOCH₃ |
| 199 | H | CH₃ | OCH₂—COOC₂H₅ |
| 200 | H | CH₃ | OCH(CH₃)—COOCH₃ |
| 201 | H | CH₃ | OCH(CH₃)—COOC₂H₅ |
| 202 | H | CH₃ | OCH₂—CF₃ |
| 203 | H | CH₃ | Ocyclopropyl |
| 204 | H | CH₃ | OCH₂-cyclopropyl |

Preference is furthermore given to the compounds of the formula ICb (compounds IC where Q=CH, R¹=CF₃, R²=Br in which R⁴ and X—R⁵ form a chain —OCH(R¹⁵)—C(O)—NR¹⁷—) in which the variables R³, R¹⁵ and R¹⁷ together have the meanings given in each case in one row of Table 2 (compounds ICb.1-ICb.204).

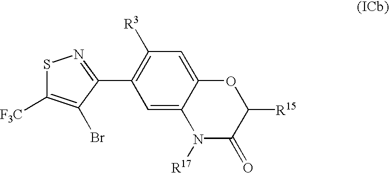

Preference is furthermore given to the compounds of the formula ICc (compounds IC where Q=CH, R¹=OCHF₂, R²=Cl in which R⁴ and X—R⁵ form a chain —OCH(R¹⁵)—C(O)—NR¹⁷—) in which the variables R³, R¹⁵ and R¹⁷ together have the meanings given in each case in one row of Table 2 (compounds ICc.1-ICc.204).

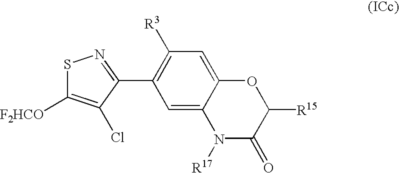

Preference is furthermore given to the compounds of the formula ICd (compounds IC where Q=CH, R¹=OCHF₂, R²=Br in which R⁴ and X—R⁵ form a chain —OCH(R¹⁵)—C(O)—NR¹⁷—) in which the variables R³, R¹⁵ and R¹⁷ together have the meanings given in each case in one row of Table 2 (compounds ICd.1-ICd.204).

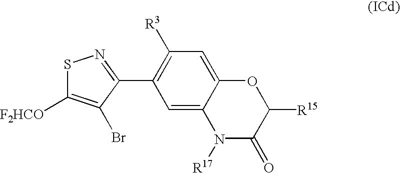

Preference is furthermore given to the compounds of the formula ICe (compounds IC where Q=CH, R¹=SO₂CH₃, R²=Cl in which R⁴ and X—R⁵ form a chain —OCH(R¹⁵)—C(O)—NR¹⁷—) in which the variables R³, R¹⁵ and R¹⁷ together have the meanings given in each case in one row of Table 2 (compounds ICe.1-ICe.204).

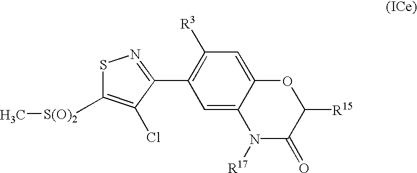

Preference is furthermore given to the compounds of the formula ICf (compounds IC where Q=CH, R¹=OSO₂CH₃, R²=Cl in which R⁴ and X—R⁵ form a chain —OCH(R¹⁵)—C(O)—NR¹⁷—) in which the variables R³, R¹⁵ and R¹⁷ together have the meanings given in each case in one row of Table 2 (compounds ICf.1-ICf.204).

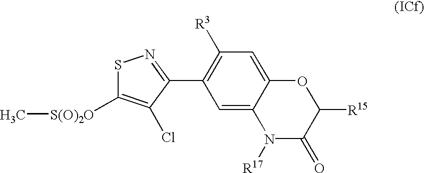

Examples of preferred compounds ID are the compounds of the formula IDa (compounds ID where Q=C—R⁶, R¹=CF₃ and R²=Cl in which R⁶ and X—R⁵ form a chain —O—C(R¹⁸)═N—) in which the variables R³, R⁴ and R¹⁸ together have the meanings given in each case in one row of Table 3 (compounds IDa.1-IDa.312).

TABLE 3

| | |
|---|---|
| | (IDa) |
| 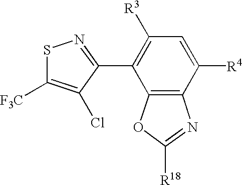 | |

| No. | R³ | R⁴ | R¹⁸ |
|---|---|---|---|
| 1 | F | Cl | H |
| 2 | F | Cl | CH₃ |
| 3 | F | Cl | C₂H₅ |
| 4 | F | Cl | n-C₃H₇ |
| 5 | F | Cl | CH(CH₃)₂ |
| 6 | F | Cl | n-C₄H₉ |
| 7 | F | Cl | CH(CH₃)—C₂H₅ |
| 8 | F | Cl | CH₂—CH(CH₃)₂ |
| 9 | F | Cl | C(CH₃)₃ |
| 10 | F | Cl | CH₂—CH═CH₂ |
| 11 | F | Cl | CH₂—C≡CH |
| 12 | F | Cl | CH₂Cl |
| 13 | F | Cl | CF₃ |
| 14 | F | Cl | CH₂-cyclopropyl |
| 15 | F | Cl | cyclopropyl |
| 16 | F | Cl | cyclopentyl |
| 17 | F | Cl | cyclohexyl |
| 18 | F | Cl | tetrahydropyran-3-yl |
| 19 | F | Cl | tetrahydropyran-4-yl |
| 20 | F | Cl | tetrahydrothiopyran-3-yl |
| 21 | F | Cl | tetrahydrothiopyran-4-yl |
| 22 | F | Cl | phenyl |
| 23 | F | Cl | CH₂—COOCH₃ |
| 24 | F | Cl | CH₂—COOC₂H₅ |
| 25 | F | Cl | CH₂—CH₂—COOCH₃ |
| 26 | F | Cl | CH₂—CH₂—COOC₂H₅ |
| 27 | F | Cl | F |
| 28 | F | Cl | Cl |
| 29 | F | Cl | Br |
| 30 | F | Cl | OCH₃ |
| 31 | F | Cl | OCH₂CH₃ |
| 32 | F | Cl | O-n-C₃H₇ |
| 33 | F | Cl | OCH(CH₃)₂ |
| 34 | F | Cl | OCH₂—CH═CH₂ |
| 35 | F | Cl | OCH₂—C≡CH |
| 36 | F | Cl | OCH₂—COOCH₃ |
| 37 | F | Cl | OCH₂—COOC₂H₅ |
| 38 | F | Cl | OCH(CH₃)—COOCH₃ |
| 39 | F | Cl | OCH(CH₃)—COOC₂H₅ |
| 40 | F | Cl | NH₂ |
| 41 | F | Cl | N(CH₃)₂ |
| 42 | F | Cl | SCH₃ |
| 43 | F | Cl | SCH₂CH₃ |
| 44 | F | Cl | S-n-C₃H₇ |
| 45 | F | Cl | SCH(CH₃)₂ |
| 46 | F | Cl | SCH₂—CH═CH₂ |
| 47 | F | Cl | SCH₂—C≡CH |
| 48 | F | Cl | SCH₂—COOCH₃ |
| 49 | F | Cl | SCH₂—COOC₂H₅ |
| 50 | F | Cl | SCH(CH₃)—COOCH₃ |
| 51 | F | Cl | COOCH₃ |
| 52 | F | Cl | COOC₂H₅ |
| 53 | Cl | Cl | H |
| 54 | Cl | Cl | CH₃ |
| 55 | Cl | Cl | C₂H₅ |
| 56 | Cl | Cl | n-C₃H₇ |
| 57 | Cl | Cl | CH(CH₃)₂ |
| 58 | Cl | Cl | n-C₄H₉ |
| 59 | Cl | Cl | CH(CH₃)—C₂H₅ |
| 60 | Cl | Cl | CH₂—CH(CH₃)₂ |
| 61 | Cl | Cl | C(CH₃)₃ |
| 62 | Cl | Cl | CH₂—CH═CH₂ |
| 63 | Cl | Cl | CH₂—C≡CH |
| 64 | Cl | Cl | CH₂Cl |
| 65 | Cl | Cl | CF₃ |
| 66 | Cl | Cl | CH₂-cyclopropyl |
| 67 | Cl | Cl | cyclopropyl |
| 68 | Cl | Cl | cyclopentyl |
| 69 | Cl | Cl | cyclohexyl |
| 70 | Cl | Cl | tetrahydropyran-3-yl |
| 71 | Cl | Cl | tetrahydropyran-4-yl |
| 72 | Cl | Cl | tetrahydrothiopyran-3-yl |
| 73 | Cl | Cl | tetrahydrothiopyran-4-yl |
| 74 | Cl | Cl | phenyl |
| 75 | Cl | Cl | CH₂—COOCH₃ |
| 76 | Cl | Cl | CH₂—COOC₂H₅ |
| 77 | Cl | Cl | CH₂—CH₂—COOCH₃ |
| 78 | Cl | Cl | CH₂—CH₂—COOC₂H₅ |
| 79 | Cl | Cl | F |
| 80 | Cl | Cl | Cl |
| 81 | Cl | Cl | Br |
| 82 | Cl | Cl | OCH₃ |
| 83 | Cl | Cl | OCH₂CH₃ |
| 84 | Cl | Cl | O-n-C₃H₇ |
| 85 | Cl | Cl | OCH(CH₃)₂ |
| 86 | Cl | Cl | OCH₂—CH═CH₂ |
| 87 | Cl | Cl | OCH₂—C≡CH |
| 88 | Cl | Cl | OCH₂—COOCH₃ |
| 89 | Cl | Cl | OCH₂—COOC₂H₅ |
| 90 | Cl | Cl | OCH(CH₃)—COOCH₃ |
| 91 | Cl | Cl | OCH(CH₃)—COOC₂H₅ |
| 92 | Cl | Cl | NH₂ |
| 93 | Cl | Cl | N(CH₃)₂ |
| 94 | Cl | Cl | SCH₃ |
| 95 | Cl | Cl | SCH₂CH₃ |
| 96 | Cl | Cl | S-n-C₃H₇ |
| 97 | Cl | Cl | SCH(CH₃)₂ |
| 98 | Cl | Cl | SCH₂—CH═CH₂ |
| 99 | Cl | Cl | SCH₂—C≡CH |
| 100 | Cl | Cl | SCH₂—COOCH₃ |
| 101 | Cl | Cl | SCH₂—COOC₂H₅ |
| 102 | Cl | Cl | SCH(CH₃)—COOCH₃ |
| 103 | Cl | Cl | COOCH₃ |
| 104 | Cl | Cl | COOC₂H₅ |
| 105 | H | Cl | H |
| 106 | H | Cl | CH₃ |
| 107 | H | Cl | C₂H₅ |
| 108 | H | Cl | n-C₃H₇ |
| 109 | H | Cl | CH(CH₃)₂ |
| 110 | H | Cl | n-C₄H₉ |
| 111 | H | Cl | CH(CH₃)—C₂H₅ |
| 112 | H | Cl | CH₂—CH(CH₃)₂ |
| 113 | H | Cl | C(CH₃)₃ |
| 114 | H | Cl | CH₂—CH═CH₂ |
| 115 | H | Cl | CH₂—C≡CH |
| 116 | H | Cl | CH₂Cl |
| 117 | H | Cl | CF₃ |
| 118 | H | Cl | CH₂-cyclopropyl |
| 119 | H | Cl | cyclopropyl |
| 120 | H | Cl | cyclopentyl |
| 121 | H | Cl | cyclohexyl |
| 122 | H | Cl | tetrahydropyran-3-yl |
| 123 | H | Cl | tetrahydropyran-4-yl |
| 124 | H | Cl | tetrahydrothiopyran-3-yl |
| 125 | H | Cl | tetrahydrothiopyran-4-yl |
| 126 | H | Cl | phenyl |
| 127 | H | Cl | CH₂—COOCH₃ |
| 128 | H | Cl | CH₂—COOC₂H₅ |
| 129 | H | Cl | CH₂—CH₂—COOCH₃ |
| 130 | H | Cl | CH₂—CH₂—COOC₂H₅ |
| 131 | H | Cl | F |
| 132 | H | Cl | Cl |
| 133 | H | Cl | Br |
| 134 | H | Cl | OCH₃ |
| 135 | H | Cl | OCH₂CH₃ |
| 136 | H | Cl | O-n-C₃H₇ |
| 137 | H | Cl | OCH(CH₃)₂ |
| 138 | H | Cl | OCH₂—CH═CH₂ |
| 139 | H | Cl | OCH₂—C≡CH |
| 140 | H | Cl | OCH₂—COOCH₃ |
| 141 | H | Cl | OCH₂—COOC₂H₅ |
| 142 | H | Cl | OCH(CH₃)—COOCH₃ |
| 143 | H | Cl | OCH(CH₃)—COOC₂H₅ |
| 144 | H | Cl | NH₂ |
| 145 | H | Cl | N(CH₃)₂ |
| 146 | H | Cl | SCH₃ |
| 147 | H | Cl | SCH₂CH₃ |
| 148 | H | Cl | S-n-C₃H₇ |
| 149 | H | Cl | SCH(CH₃)₂ |
| 150 | H | Cl | SCH₂—CH═CH₂ |
| 151 | H | Cl | SCH₂—C≡CH |
| 152 | H | Cl | SCH₂—COOCH₃ |
| 153 | H | Cl | SCH₂—COOC₂H₅ |
| 154 | H | Cl | SCH(CH₃)—COOCH₃ |
| 155 | H | Cl | COOCH₃ |
| 156 | H | Cl | COOC₂H₅ |
| 157 | F | CN | H |
| 158 | F | CN | CH₃ |
| 159 | F | CN | C₂H₅ |
| 160 | F | CN | n-C₃H₇ |
| 161 | F | CN | CH(CH₃)₂ |
| 162 | F | CN | n-C₄H₉ |
| 163 | F | CN | CH(CH₃)—C₂H₅ |
| 164 | F | CN | CH₂—CH(CH₃)₂ |
| 165 | F | CN | C(CH₃)₃ |
| 166 | F | CN | CH₂—CH═CH₂ |
| 167 | F | CN | CH₂—C≡CH |
| 168 | F | CN | CH₂Cl |
| 169 | F | CN | CF₃ |
| 170 | F | CN | CH₂-cyclopropyl |
| 171 | F | CN | cyclopropyl |
| 172 | F | CN | cyclopentyl |
| 173 | F | CN | cyclohexyl |
| 174 | F | CN | tetrahydropyran-3-yl |
| 175 | F | CN | tetrahydropyran-4-yl |
| 176 | F | CN | tetrahydrothiopyran-3-yl |
| 177 | F | CN | tetrahydrothiopyran-4-yl |
| 178 | F | CN | phenyl |
| 179 | F | CN | CH₂—COOCH₃ |
| 180 | F | CN | CH₂—COOC₂H₅ |
| 181 | F | CN | CH₂—CH₂—COOCH₃ |
| 182 | F | CN | CH₂—CH₂—COOC₂H₅ |
| 183 | F | CN | F |
| 184 | F | CN | Cl |
| 185 | F | CN | Br |
| 186 | F | CN | OCH₃ |
| 187 | F | CN | OCH₂CH₃ |
| 188 | F | CN | O-n-C₃H₇ |
| 189 | F | CN | OCH(CH₃)₂ |
| 190 | F | CN | OCH₂—CH═CH₂ |
| 191 | F | CN | OCH₂—C≡CH |
| 192 | F | CN | OCH₂—COOCH₃ |
| 193 | F | CN | OCH₂—COOC₂H₅ |
| 194 | F | CN | OCH(CH₃)—COOCH₃ |
| 195 | F | CN | OCH(CH₃)—COOC₂H₅ |
| 196 | F | CN | NH₂ |
| 197 | F | CN | N(CH₃)₂ |
| 198 | F | CN | SCH₃ |
| 199 | F | CN | SCH₂CH₃ |
| 200 | F | CN | S-n-C₃H₇ |
| 201 | F | CN | SCH(CH₃)₂ |
| 202 | F | CN | SCH₂—CH═CH₂ |
| 203 | F | CN | SCH₂—C≡CH |
| 204 | F | CN | SCH₂—COOCH₃ |
| 205 | F | CN | SCH₂—COOC₂H₅ |
| 206 | F | CN | SCH(CH₃)—COOCH₃ |
| 207 | F | CN | COOCH₃ |
| 208 | F | CN | COOC₂H₅ |
| 209 | Cl | CN | H |
| 210 | Cl | CN | CH₃ |
| 211 | Cl | CN | C₂H₅ |
| 212 | Cl | CN | n-C₃H₇ |
| 213 | Cl | CN | CH(CH₃)₂ |
| 214 | Cl | CN | n-C₄H₉ |
| 215 | Cl | CN | CH(CH₃)—C₂H₅ |
| 216 | Cl | CN | CH₂—CH(CH₃)₂ |
| 217 | Cl | CN | C(CH₃)₃ |
| 218 | Cl | CN | CH₂—CH═CH₂ |
| 219 | Cl | CN | CH₂—C≡CH |
| 220 | Cl | CN | CH₂Cl |
| 221 | Cl | CN | CF₃ |
| 222 | Cl | CN | CH₂-cyclopropyl |
| 223 | Cl | CN | cyclopropyl |
| 224 | Cl | CN | cyclopentyl |
| 225 | Cl | CN | cyclohexyl |
| 226 | Cl | CN | tetrahydropyran-3-yl |
| 227 | Cl | CN | tetrahydropyran-4-yl |
| 228 | Cl | CN | tetrahydrothiopyran-3-yl |
| 229 | Cl | CN | tetrahydrothiopyran-4-yl |
| 230 | Cl | CN | phenyl |
| 231 | Cl | CN | CH₂—COOCH₃ |
| 232 | Cl | CN | CH₂—COOC₂H₅ |
| 233 | Cl | CN | CH₂—CH₂—COOCH₃ |
| 234 | Cl | CN | CH₂—CH₂—COOC₂H₅ |
| 235 | Cl | CN | F |
| 236 | Cl | CN | Cl |
| 237 | Cl | CN | Br |
| 238 | Cl | CN | OCH₃ |
| 239 | Cl | CN | OCH₂CH₃ |
| 240 | Cl | CN | O-n-C₃H₇ |
| 241 | Cl | CN | OCH(CH₃)₂ |
| 242 | Cl | CN | OCH₂—CH═CH₂ |
| 243 | Cl | CN | OCH₂—C≡CH |
| 244 | Cl | CN | OCH₂—COOCH₃ |
| 245 | Cl | CN | OCH₂—COOC₂H₅ |
| 246 | Cl | CN | OCH(CH₃)—COOCH₃ |
| 247 | Cl | CN | OCH(CH₃)—COOC₂H₅ |
| 248 | Cl | CN | NH₂ |
| 249 | Cl | CN | N(CH₃)₂ |
| 250 | Cl | CN | SCH₃ |
| 251 | Cl | CN | SCH₂CH₃ |
| 252 | Cl | CN | S-n-C₃H₇ |
| 253 | Cl | CN | SCH(CH₃)₂ |
| 254 | Cl | CN | SCH₂—CH═CH₂ |
| 255 | Cl | CN | SCH₂—C≡CH |
| 256 | Cl | CN | SCH₂—COOCH₃ |
| 257 | Cl | CN | SCH₂—COOC₂H₅ |
| 258 | Cl | CN | SCH(CH₃)—COOCH₃ |
| 259 | Cl | CN | COOCH₃ |
| 260 | Cl | CN | COOC₂H₅ |
| 261 | H | CN | H |
| 262 | H | CN | CH₃ |
| 263 | H | CN | C₂H₅ |
| 264 | H | CN | n-C₃H₇ |
| 265 | H | CN | CH(CH₃)₂ |
| 266 | H | CN | n-C₄H₉ |
| 267 | H | CN | CH(CH₃)—C₂H₅ |
| 268 | H | CN | CH₂—CH(CH₃)₂ |
| 269 | H | CN | C(CH₃)₃ |
| 270 | H | CN | CH₂—CH═CH₂ |
| 271 | H | CN | CH₂—C≡CH |
| 272 | H | CN | CH₂Cl |
| 273 | H | CN | CF₃ |
| 274 | H | CN | CH₂-cyclopropyl |
| 275 | H | CN | cyclopropyl |
| 276 | H | CN | cyclopentyl |
| 277 | H | CN | cyclohexyl |
| 278 | H | CN | tetrahydropyran-3-yl |
| 279 | H | CN | tetrahydropyran-4-yl |
| 280 | H | CN | tetrahydrothiopyran-3-yl |
| 281 | H | CN | tetrahydrothiopyran-4-yl |
| 282 | H | CN | phenyl |
| 283 | H | CN | CH₂—COOCH₃ |
| 284 | H | CN | CH₂—COOC₂H₅ |
| 285 | H | CN | CH₂—CH₂—COOCH₃ |
| 286 | H | CN | CH₂—CH₂—COOC₂H₅ |
| 287 | H | CN | F |
| 288 | H | CN | Cl |
| 289 | H | CN | Br |
| 290 | H | CN | OCH₃ |
| 291 | H | CN | OCH₂CH₃ |
| 292 | H | CN | O-n-C₃H₇ |
| 293 | H | CN | OCH(CH₃)₂ |
| 294 | H | CN | OCH₂—CH═CH₂ |
| 295 | H | CN | OCH₂—C≡CH |
| 296 | H | CN | OCH₂—COOCH₃ |
| 297 | H | CN | OCH₂—COOC₂H₅ |
| 298 | H | CN | OCH(CH₃)—COOCH₃ |
| 299 | H | CN | OCH(CH₃)—COOC₂H₅ |
| 300 | H | CN | NH₂ |
| 301 | H | CN | N(CH₃)₂ |
| 302 | H | CN | SCH₃ |
| 303 | H | CN | SCH₂CH₃ |
| 304 | H | CN | S-n-C₃H₇ |
| 305 | H | CN | SCH(CH₃)₂ |
| 306 | H | CN | SCH₂—CH═CH₂ |
| 307 | H | CN | SCH₂—C≡CH |
| 308 | H | CN | SCH₂—COOCH₃ |
| 309 | H | CN | SCH₂—COOC₂H₅ |
| 310 | H | CN | SCH(CH₃)—COOCH₃ |
| 311 | H | CN | COOCH₃ |
| 312 | H | CN | COOC₂H₅ |

Preference is furthermore given to the compounds of the formula IDb (compounds ID where Q=C—R⁶, R¹=CF₃ and R²=Br in which R⁶ and X—R⁵ form a chain —O—C(R¹⁸)═N—) in which the variables R³, R⁴ and R¹⁸ together have the meanings given in each case in one row of Table 3 (compounds IDb.1-IDb.312).

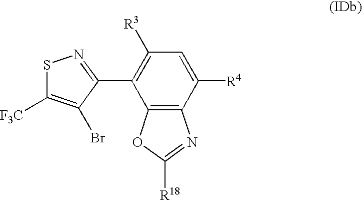

Preference is furthermore given to the compounds of the formula IDc (compounds ID where Q=C—R⁶, R¹=OCHF₂ and R²=Cl in which R⁶ and X—R⁵ form a chain —O—C(R¹⁸)═N—) in which the variables R³, R⁴ and R¹⁸ together have the meanings given in each case in one row of Table 3 (compounds IDc.1-IDc.312).

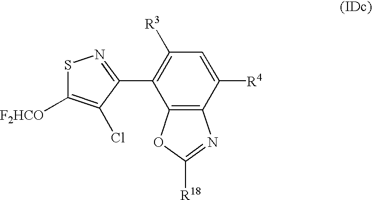

Preference is furthermore given to the compounds of the formula IDd (compounds ID where Q=C—R⁶, R¹=OCHF₂ and R²=Br in which R⁶ and X—R⁵ form a chain —O—C(R¹⁸)═N—) in which the variables R³, R⁴ and R¹⁸ together have the meanings given in each case in one row of Table 3 (compounds IDd.1-IDd.312).

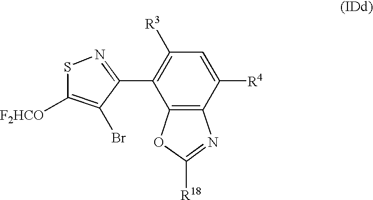

Preference is furthermore given to the compounds of the formula IDe (compounds ID where Q=C—R⁶, R¹=SO₂CH₃ and R²=Cl in which R⁶ and X—R⁵ form a chain —O—C(R¹⁸)═N—) in which the variables R³, R⁴ and R¹⁸ together have the meanings given in each case in one row of Table 3 (compounds IDe.1-IDe.312).

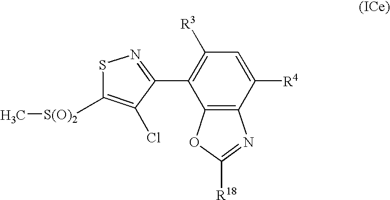

Preference is furthermore given to the compounds of the formula IDf (compounds ID where Q=C—R⁶, R¹=OSO₂CH₃ and R²=Cl in which R⁶ and X—R⁵ form a chain —O—C(R¹⁸)═N—) in which the variables R³, R⁴ and R¹⁸ together have the meanings given in each case in one row of Table 3 (compounds IDf.1-IDf.312).

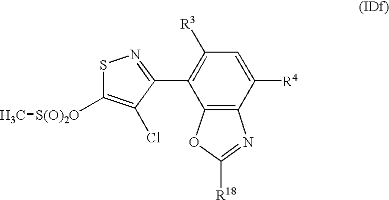

Preference is furthermore given to the compounds of the formula IDg (compounds ID where Q=C—R⁶, R¹=CF₃ and R²=Cl in which R⁶ and X—R⁵ form a chain —S—C(R¹⁸)═N—) in which the variables R³, R⁴ and R¹⁸ together have the meanings given in each case in one row of Table 3 (compounds IDg.1-IDg.312).

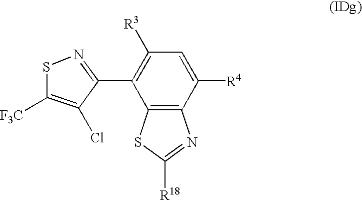

Preference is furthermore given to the compounds of the formula IDh (compounds ID where Q=C—R⁶, R¹=CF₃ and R²=Br in which R⁶ and X—R⁵ form a chain —S—C(R¹⁸)═N—) in which the variables R³, R⁴ and R¹⁸ together have the meanings given in each case in one row of Table 3 (compounds IDh.1-IDh.312).

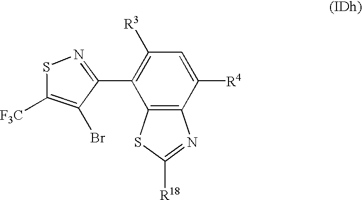

Preference is furthermore given to the compounds of the formula IDi (compounds ID where Q=C—R⁶, R¹=OCHF₂ and R²=Cl in which R⁶ and X—R⁵ form a chain —S—C(R¹⁸)═N—) in which the variables R³, R⁴ and R¹⁸ together have the meanings given in each case in one row of Table 3 (compounds IDi.1-IDi.312).

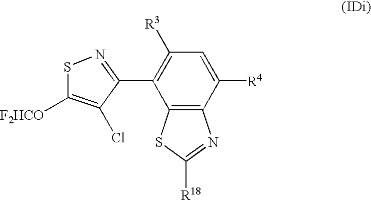

Preference is furthermore given to the compounds of the formula IDk (compounds ID where Q=C—R⁶, R¹=OCHF₂ and R²=Br in which R⁶ and X—R⁵ form a chain —S—C(R¹⁸)═N—) in which the variables R³, R⁴ and R¹⁸ together have the meanings given in each case in one row of Table 3 (compounds IDk.1-IDk.312).

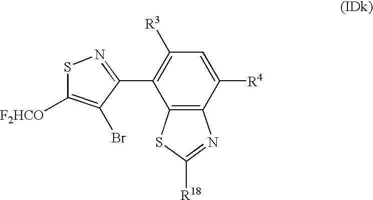

Preference is furthermore given to the compounds of the formula IDl (compounds ID where Q=C—R⁶, R¹=SO₂CH₃ and R²=Cl in which R⁶ and X—R⁵ form a chain —S—C(R¹⁸)═N—) in which the variables R³, R⁴ and R¹⁸ together have the meanings given in each case in one row of Table 3 (compounds IDl.1-IDl.312).

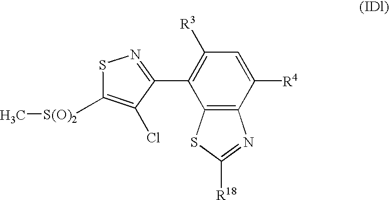

Preference is furthermore given to the compounds of the formula IDm (compounds ID where Q=C—R⁶, R¹=OSO₂CH₃ and R²=Cl in which R⁶ and X—R⁵ form a chain —S—C(R¹⁸)═N—) in which the variables R³, R⁴ and R¹⁸ together have the meanings given in each case in one row of Table 3 (compounds IDm.1-IDm.312).

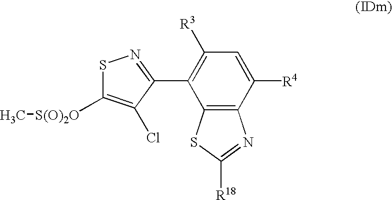

The 3-arylisothiazoles of the formula I according to the invention can be prepared similarly to known processes for the preparation of 3-arylisothiazoles and in particular by the synthesis routes described below. Hereinbelow, “aryl” denotes a radical of the formula:

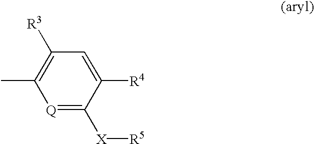

and “hetaryl” denotes a radical of the formula:

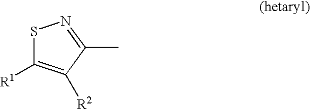

in which R¹ to R⁵, X and Q are as defined above.

A) The compounds of the formula I can be prepared, for example, by constructing the isothiazole ring from suitably substituted aryl compounds.

A1 One example is the construction of 4-amino-3-arylisothiazoles of the formula A4 from benzylnitriles of the formula A1 according to the reaction sequence below:

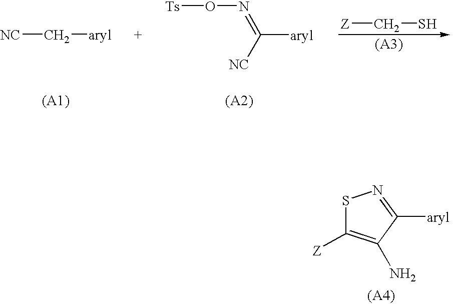

The radical Z in the 4-amino-3-arylisothiazole A4 is then converted by standard methods into the substituent R¹. Conversion of the NH₂ group into another substituent R² is also possible by standard methods. In principle, it is immaterial whether the amino group in A4 is first converted into another substituent R² giving a compound A5 or whether the group Z is converted into a substituent R¹ giving a compound A5′.

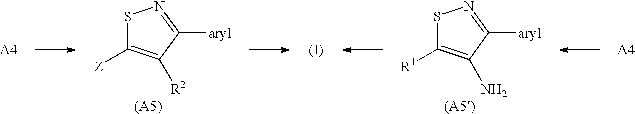

To construct the thiazole ring, a benzylnitrile of the formula A1 is initially nitrosated in the presence of a base with a nitrosating agent, for example an alkyl nitrite, such as isoamyl nitrite, and then converted into the tosyl oxime A2 using tosyl chloride. In the formulae A1, A2 and A4, aryl is as defined above. Ts in formula A2 denotes a tosyl group (═CH₃—C₆H₄—SO₂—). The tosyl oxime A2 is then in the presence of a base reacted with a mercaptan of the formula A3 in which Z is an electron-withdrawing radical, for example a carboxy-C₁-C₄-alkyl- or cyano radical, to give the 3-arylisothiazole of the formula A4. Examples of suitable compounds A3 are the C₁-C₄-alkyl thioglycolates.

Suitable bases for the nitrosation of A1 are, for example: alkali metal hydroxides, e.g. sodium hydroxide, alkali metal carbonates, such as potassium carbonate and sodium carbonate, alkali metal alkoxides, such as sodium ethoxide, alkali metal hydrides, such as sodium hydride, and tertiary amines, such as triethylamine. Suitable bases for the reaction of A2 with A3 to give A4 are, for example: nitrogen bases, such as pyridine and morpholine, or alkali metal alkoxides, such as sodium ethoxide.

The above reaction sequence has been described in the literature for the preparation of 4-amino-3-arylisothiazole-5-carboxylic esters (compounds A4 where Z=alkyloxycarbonyl), for example by J. Beck et al. in U.S. Pat. No. 4,544,752, U.S. Pat. No. 4,346,094, J. Heterocyclic Chem. 24 (1987), 243; and K. Gewald et al, Liebigs Ann. Chem. 1979, 1534-1546.

The benzylnitriles used as starting materials can be prepared by processes known per se from the literature from a corresponding benzoic acid compound A6, for example by the reaction sequence below:

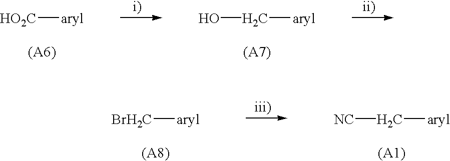

i reduction of A6 to the benzyl alcohol A7, for example by reacting A6 with a borane complex such as BH₃—S(CH₃)₂ in an inert organic solvent, for example an ether such as diethyl ether or tetrahydrofuran or in a halogenated hydrocarbon such as dichloromethane or in a mixture of the above solvents;

ii halogenation of A7 to give benzyl bromide A8, for example by reacting A7 with CBr₄/PPh₃ in one of the solvents mentioned above, and subsequent

iii reaction of the bromide A8 in the sense of a Kolbe nitrile synthesis with NaCN in an org. solvent, for example in acetone, ethanol or triethylene glycol.

The conversion of the amino function in the 4-position of the isothiazole ring of A4 or A5′ into other substituents R² can be carried out, for example, using the synthesis sequence described below:

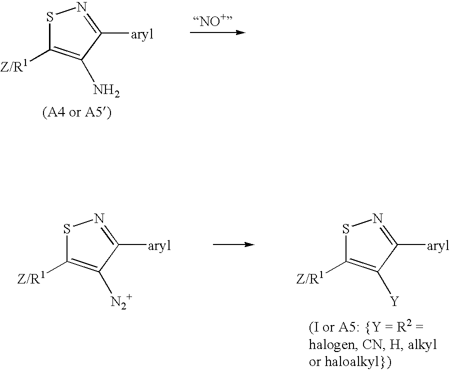

In the literature, this reaction sequence has already been described for 3-phenylisothiazole-5-carboxylic acids (see J. Beck et al. U.S. Pat. No. ,454,4752 and U.S. Pat. No. 4,346,094; J. Beck et al, J. Heterocyclic Chem. 24 (1987), 243). What has been said under C1 with respect to the conversion of XR⁵=NH₂ also applies to the above reaction sequence.

Here, the amino group in the 4-position of the isothiazole ring of A4 or A5′ is initially converted into a diazonium group using a nitrosating agent “NO⁺”. The resulting diazonium group is then converted in a customary manner, it being possible to generate the radicals R² listed below:

R²=cyano or halogen {for example by the Sandmeyer reaction: cf., for example, Houben-Weyl, Methoden der Organischen Chemie [Methods of Organic Chemistry], Georg Thieme Verlag Stuttgart, Vol. 5/4, 4th edition 1960, p. 438ff.},

R²=alkyl or haloalkyl by reaction with alkenes or haloalkenes in the sense of a Meerwein arylation; cf., for example, C. S. Rondestredt, Org. React. 11 (1960), 189 and H. P. Doyle et al., J. Org. Chem. 42 (1977), 2431}.

Suitable nitrosating agents are: nitrosonium tetrafluoroborate, nitrosyl chloride, nitrosyl sulfuric acid, alkyl nitrites, such as, for example, t-butyl nitrite, or salts of nitrous acid, such as, for example, sodium nitrite.

The halogen compounds I or A5 {R²=halogen} for their part can then be converted into other radicals R², for example into a cyano group by conversion with copper(I) cyanide, analogously to T. Naito et al. in Chem. Pharm. Bull. 16 (1968), pp. 148-159.

If Z in formula A4 or A5 is a carboxyalkyl group, the corresponding trifluoromethyl compound (compounds I where R¹=trifluoromethyl) can be obtained in a simple manner. To this end, an isothiazolecarboxylic ester of the formula A4 or A5 is hydrolyzed to give the corresponding isothiazolecarboxylic acid of the formula II

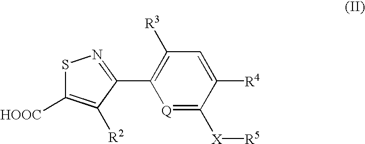

in which the variables X, Q, R², R³, R⁴, R⁵ are as defined in claim 1 (compounds of the formula A4 or A5 where Z=COOH). The carboxylic acid II is then reacted with a fluorinating agent. This conversion can be achieved, for example, by treating the carboxylic acid in an autoclave with SF₄/HF with heating, preferably at temperatures in the range from 40 to 100° C., for example according to T. Nickson, J. Fluorine Chem. 55 (2) (1991), 173-177. This process can preferably be used for preparing compounds I according to the invention where R²=halogen.

A2) A further route for constructing 3-arylisothiazoles follows the synthesis of 5-amino-3-arylisothiazoles described by Goerdelar et al. (Chem. Ber. 94 (1961), p. 2950) which is shown in the scheme below (see also T. Naito et al., Chem. Pharm. Bull. 16 (1) (1968), p. 148-159):

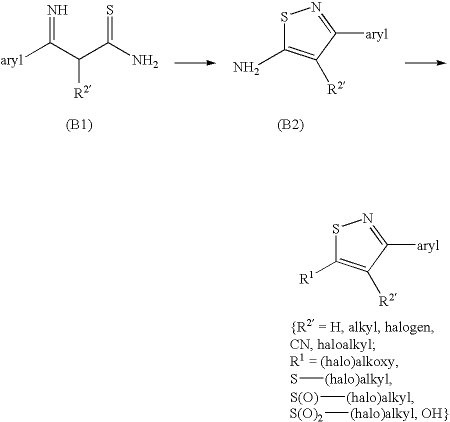

Here, initially a 5-amino-3-arylisothiazole B2 is prepared by cyclization of a β-iminothioamide of the formula B1. B2 is then used to prepare a compound of the formula B3 according to the invention, by converting the amino group in the 5-position of the isothiazole ring. In the compounds B1 and B2, R^{2′} is hydrogen, C₁-C₄-alkyl or C₁-C₄-haloalkyl, preferably hydrogen.

If R^{2′} in B2 is hydrogen, the group R^{2′} can, prior to the conversion of the 5-amino group into a group R¹, also be converted into a halogen atom (cf. T. Naito et al., Chem. Pharm. Bull. 16 (1) (1968), 148-159, and the halogenation of the 4-position of the isothiazole moiety of I described below under B).

The conversion of the amino group located in the 5-position of the isothiazole ring can be carried out similarly to the procedure described under A1 for converting the amino group located in the 4-position of the isothiazole ring of A4 or A5′ and according to the procedure for converting the amino group NH₂=X—R⁵ described under C1. The conversion is initiated by nitrosation of the amino group in the 5-position of the isothiazole ring. The resulting diazonium compound is then converted further as follows:

R¹=alkoxy or haloalkoxy: conversion of the diazonium group into hydroxyl {for example by decomposition to phenol: cf., for example, Org. Synth. Coll. Vol. 3 (1955), p. 130}. The hydroxyl compound is then, in the sense of an ether synthesis, converted by reaction with an alkyl halide into the corresponding alkoxy or haloalkoxy compound. It is also possible to convert the hydroxyl group by reaction with (halo)alkylsulfonyl chloride into the corresponding (halo)alkylsulfonyloxy group.

R¹=mercapto, C₁-C₆-alkylthio or haloalkylthio {cf., for example, Houben-Weyl, Methoden der Organischen Chemie, Georg Thieme Verlag Stuttgart, Vol. E11 1984, pp. 43 and 176}. The mercapto group is then, in the sense of a thioether synthesis, converted by reaction with an alkyl halide into an alkylthio or haloalkylthio group, for example by reaction with methyl halide into the methylthio group or by reaction with chloro- or bromodifluoromethane into the difluoromethylthio group. The alkylthio- or haloalkylthio group can then be converted by selective oxidation into the (halo)alkylsulfinyl or (halo)alkylsulfonyl group.

If group R¹ in compound B3 is S—C₁-C₄-(halo)alkyl (thioalkyl ether B3a), B3a can be converted by oxidation of the sulfur according to known processes into the corresponding sulfinylalkyl compound B3b {R¹=S(O)—C₁-C₄-(halo)alkyl} or into the corresponding sulfonyl(halo)alkyl compound B3c {R¹=S(O)₂—C₁-C₄-(halo)alkyl}. By oxidation of B3 with H₂O₂ in acetic acid or by oxidation of B3 with KMnO₄, it is possible, for example, to prepare the 5-(halo)alkylsulfonyl-4-haloisothiazoles from the 5-(halo)alkylthio-4-haloisothiazoles (cf. T. Naito, Chem. Pharm. Bull. 16 (1) (1968), 148-159).

In the sense of an ether synthesis, the hydroxyl compound B3d {B3 where R¹═OH} can be converted by reaction with alkyl halides into the compound I according to the invention where R¹=alkoxy or haloalkoxy, for example by reaction with methyl halide such as methyl iodide into the methoxy compound or by reaction with chloro- or bromodifluoromethane into the difluoromethoxy compound. The reaction is preferably carried out in the presence of a strong base.

To prepare the compounds I where R¹=difluoromethoxy, which are preferred according to the invention, the corresponding 3-aryl-5-hydroxyisothiazole B3d (R¹=hydroxyl) is, for example, reacted with chlorodifluoromethane, preferably in an organic solvent. This reaction is preferably carried out in the presence of a base. Examples of suitable bases are alkali metal hydroxides, such as sodium hydroxide or potassium hydroxide, alkali metal carbonates and bicarbonates, such as potassium carbonate or bicarbonate or sodium carbonate or bicarbonate, or an organic base, for example, an alkoxide, such as sodium methoxide or ethoxide or potassium methoxide or ethoxide, in particular tertiary amines, such as triethylamine or pyridine.

The gaseous chlorodifluoromethane is preferably introduced slowly into the reaction mixture containing the 5-hydroxyisothiazole B3d, preferably dissolved or suspended in a solvent, if appropriate a base and/or further catalysts. If the reaction is carried out under atmospheric pressure, excess chlorodifluoromethane gas is preferably trapped using a low-temperature condenser. However, the reaction can also be carried out under elevated chlorodifluoromethane pressure in a closed apparatus (autoclave) at pressures between about 0.1 and 100 bar. The reaction temperature is usually between the melting point and the boiling point of the reaction mixture, preferably at temperatures in the range from 50 to 150° C. To obtain a high yield, it may be advantageous to employ an excess of chlorodifluoromethane (based on the 5-hydroxyisothiazole B3d). The excess can, for example, be up to five times the molar amount of the 5-hydroxyisothiazole B3d used.

Suitable solvents are inert organic solvents, for example hydrocarbons, such as toluene or hexane, ethers, such as diethyl ether, dimethoxyethane, methyl-t-butyl ether, dioxane or tetrahydrofuran (THF), amides, such as dimethyl formamide (DMF), N,N-dimethylacetamide (DMA) or N-methylpyrrolidone (NMP), C₁-C₆-alkanols, such as methanol or ethanol, or else mixtures of such solvents with one another or with water. To improve conversion or to increase the reaction rate, it is frequently advantageous to add a phase-transfer catalyst, for example a tetraalkylammonium salt, such as tetrabutylammonium chloride, or a crown ether, such as 18-crown-6 or 15-crown-5, in catalytic amounts (0.01-20 mol %, based on the 5-hydroxyisothiazole).

Reaction of the hydroxy compound B3d with alkylsulfonyl halides or haloalkylsulfonyl halides such as methylsulfonyl chloride gives the corresponding (halo)alkylsulfonyloxy compound I {R¹=O—S(O)₂—C₁-C₄-(halo)alkyl}. The reaction is preferably carried out in the presence of a base such as triethylamine, pyridine or dimethylaminopyridine.

A3 The 5-haloalkylisothiazoles I {R¹=C₁-C₄-haloalkyl} can furthermore be obtained by halogenating 5-alkylisothiazoles which are not according to the invention (compounds of the formula I where R¹=C₁-C₄-alkyl, in particular methyl). The alkyl group of the 5-alkylisothiazoles can be halogenated, for example, by free-radical halogenation using, for example, chlorine, sulfuryl chloride or N-halosuccinimides, such as N-chloro- or N-bromosuccinimide. This generally gives the monohalo compound. The 5-trichloromethyl-3-arylisothiazoles can be prepared from the corresponding 5-methyl compounds by photochlorination using standard processes (for example by the methods described in Houben Weyl 5/3, Methoden der Organischen Chemie, Georg Thieme Verlag, p. 735 ff. and Organikum, 17th edition, p. 161 ff.).

The preparation of the 5-alkylisothiazoles used as starting materials is known from the literature or can be carried out similar to the methods described therein (K. Akiba et al., J. Am. Chem. Soc. 107 (1985), 2721-2730; T. Naito, Chem. Pharm. Bull. 16 (1) (1968), 148-159; M. Beringer, Helv. Chim. Acta 49 (1966), 2466-2469).

3-Aryl-5-trifluoromethylisothiazoles of the formula I can furthermore be prepared from the 5-trichloromethylisothiazoles by chlorine-fluorine exchange. The conversion is carried out, for example, by reacting the trichloromethyl compound with HF, HF/SbCl₅ or SbF₅ (see, for example, Houben-Weyl E 10a, p. 133ff; Houben-Weyl 5/3, p. 119).

A4 5-Alkylthio-4-cyanoisothiazoles can furthermore be prepared similar to a method described in the literature (see Houben-Weyl E8a, p. 686), in accordance with the scheme below.

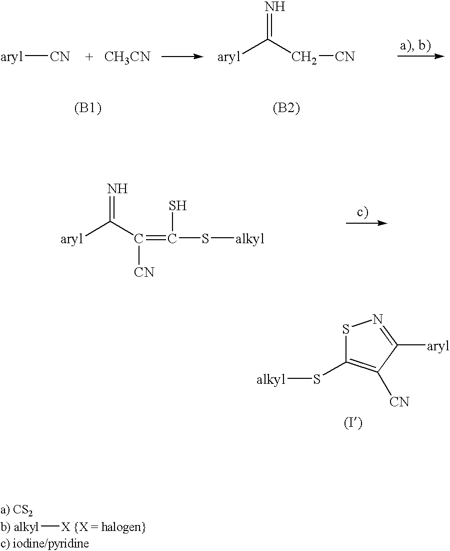

The thioalkyl group in compound I′ (compound I where R¹=S—C₁-C₄-alkyl and R²=CN) can be converted selectively into a C₁-C₄-alkylsulfinyl or an alkylsulfonyl group by oxidation, for example with KMₙO₄.

A5 3-arylisothiazoles can furthermore be prepared according to the scheme below by reacting 5-aryl-1,3,4-oxathiazoles with acetylenecarboxylic esters and subsequent conversion of the carboxylic ester group located in the 5-position of the isothiazole ring into a radical R¹. The conversion of 5-aryl-1,3,4-oxathiazoles with acetylenecarboxylic esters into 3-arylisothiazole-5-carboxylic esters has been described by R. K. Howe et al. (J. Org. Chem. 43 (1978), 3742-3745, and literature cited therein). 5-Aryl-1,3,4-oxathiazoles for their part are obtainable from arylcarboxylic acids. The arylcarboxylic acids are converted in a known manner into the carboxamide which is then reacted with chlorocarbonylsulfenyl chloride (Cl—C(O)—S—Cl) in an inert organic solvent to give the 5-aryl-1,3,4-oxathiazole.

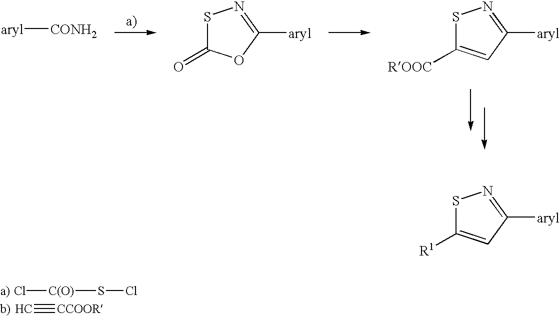

B) Moreover, 3-arylisothiazoles I can be prepared by functionalization of the 4-position of the isothiazole ring, for example by halogenation of 3-arylisothiazoles in which R² is hydrogen:

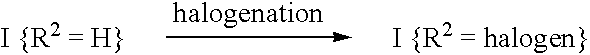

Suitable halogenating agents are, for example, fluorine, DAST (diethylaminosulfur trifluoride), chlorine, N-chlorosuccinimide, sulfuryl chloride, thionyl chloride, phosgene, phosphorus trichloride, phosphorus oxychloride, bromine, N-bromosuccinimide, phosphorus tribromide and phosphorus oxybromide. For the chlorination of isothiazoles with N-chlorosuccinimide, see also K. Ohkata et al., Heterocycles, 37 (1994), 859-868.

The reaction is usually carried out in an inert solvent/diluent, for example in a hydrocarbon, such as n-hexane and toluene, a halogenated hydrocarbon, such as dichloromethane, carbon tetrachloride and chloroform, an ether, such as methyl tert-butyl ether, an alcohol, such as methanol and ethanol, a carboxylic acid, such as acetic acid, or in a polar aprotic solvent, such as acetonitrile.

The reaction temperature is usually between the melting point and the boiling point of the reaction mixture, preferably from 0 to 100° C.

To obtain as high a yield of the product of value as possible, the halogenating agent is employed in approximately equimolar amounts or in an excess of up to five times the molar amount, based on the amount of starting material.

C) Compounds I in which Q=CH (compounds IA or IC) can be converted into other compounds IA by functionalization of the phenyl rings. Examples of this are:

C.1 Nitration of 3-arylisothiazoles IA in which XR⁵ is hydrogen, and conversion of the products of the process in the further compounds of the formula IA:

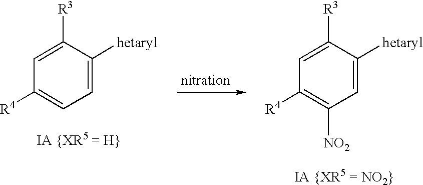

Suitable nitrating agents are, for example, nitric acid in various concentrations, including concentrated and fuming nitric acid, mixtures of sulfuric acid and nitric acid, and furthermore acetyl nitrates and alkyl nitrates.

The reaction can be carried out either without using a solvent in an excess of the nitrating agent, or in an inert solvent or diluent, suitable solvents or diluents being, for example, water, mineral acids, organic acids, halogenated hydrocarbons, such as methylene chloride, anhydrides, such as acetic anhydride, and mixtures of these.

Starting material IA {XR⁵=H} and nitrating agent are advantageously employed in approximately equimolar amounts; however, to optimize the conversion of the starting material, it may be advantageous to use an excess of nitrating agent, up to about 10 times the molar amount based on IA. When the reaction is carried out without a solvent in the nitrating agent, the latter is present in an even greater excess.

The reaction temperature is usually from −100° C. to 200° C., preferably from −30 to 50° C.

The compounds IA where XR⁵=NO₂ can then be reduced to give compounds IA where X—R⁵=NH₂ or —NHOH:

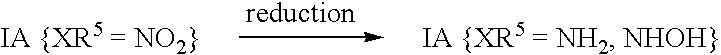

The reduction is generally carried out by reacting the nitro compound with a metal, such as iron, zinc or tin or with SnCl₂, under acidic reaction conditions, or with a complex hydride, such as lithium aluminum hydride and sodium borohydride, the reduction being carried out without dilution or in a solvent or diluent. Suitable solvents are—depending on the reducing agent chosen—for example water, alcohols, such as methanol, ethanol and isopropanol, or ethers, such as diethyl ether, methyl tert-butyl ether, dioxane, tetrahydrofuran and ethylene glycol dimethyl ether.

If the reduction is carried out using a metal, the reaction is preferably carried out without a solvent in an inorganic acid, in particular in concentrated or dilute hydrochloric acid, or in a liquid organic acid, such as acetic acid or propionic acid. However, it is also possible to dilute the acid with an inert solvent, for example one of those mentioned above. The reduction with complex hydrides is preferably carried out in a solvent, for example an ether or an alcohol.

The nitro compound IA {X—R⁵=NO₂} and the reducing agent are frequently employed in approximately equimolar amounts; to optimize the reaction it may be advantageous to use an excess of one of the two components, up to about 10 times the molar amount.

The amount of acid is not critical. To ensure as complete a reduction of the starting material as possible, it is advantageous to use at least an equivalent amount of acid.

Frequently, an excess of acid, based on IA {X—R⁵=NO₂}, is employed.

The reaction temperature is usually in the range from −30° C. to 200° C., preferably in the range from 0° C. to 80° C.

For work-up, the reaction mixture is usually diluted with water, and the product is isolated by filtration, crystallization or extraction with a substantially water-immiscible solvent, for example ethyl acetate, diethyl ether or methylene chloride. If desired, the product can then be purified in a conventional manner.

The nitro group of the compounds IA {X—R⁵=NO₂} can also be hydrogenated catalytically using hydrogen. Catalysts suitable for this purpose are, for example, Raney nickel, palladium on carbon, palladium oxide, platinum and platinum oxide, an amount of catalyst of from 0.05 to 10.0 mol %, based on the compound to be reduced, generally being sufficient.

The reaction is carried out either without a solvent or in an inert solvent or diluent, for example in acetic acid, a mixture of acetic acid and water, ethyl acetate, ethanol or in toluene.

After removal of the catalyst, the reaction solution can be worked up in a conventional manner to afford the product.

The hydrogenation can be carried out under atmospheric hydrogen pressure or under elevated hydrogen pressure.

The amino group in IA {X—R⁵=NH₂} can then be diazotized in a conventional manner. The diazonium salts then give access to the compounds I where:

X—R⁵=cyano or halogen {for example by the Sandmeyer reaction: cf., for example, Houben-Weyl, Methoden der Organischen Chemie, Georg Thieme Verlag Stuttgart, Vol. 5/4, 4th edition 1960, p. 438ff.},

X—R⁵=hydroxyl {for example by generating phenols by heating diazonium salts: cf., for example, Org. Synth. Coll. Vol. 3 (1955), p. 130},

X—R⁵=mercapto or C₁-C₆-alkylthio {cf., for example, Houben-Weyl, Methoden der Organischen Chemie, Georg Thieme Verlag Stuttgart, Vol. E11 1984, pp. 43 and 176},

X—R⁵=halosulfonyl {cf., for example, Houben-Weyl, Methoden der Organischen Chemie, Georg Thieme Verlag Stuttgart, Vol. E11 1984, p. 1069f.},

X—R⁵=for example —CH₂—CH(halogen)-CO—O—Y—R⁷, —CH=C(halogen)-CO—O—Y—R⁷, —CH₂—CH(halogen)-PO—(O—Y—R⁷)₂, —CH=C(halogen)-CO—(O—Y—R⁷)₂ {these are generally products of a Meerwein arylation; cf., for example, C. S. Rondestredt, Org. React. 11 (1960), 189 and H. P. Doyle et al., J. Org. Chem. 42 (1977), 2431}.

The diazonium salt in question of IA {X—R⁵=N₂⁺} is generally prepared in a manner known per se by reacting IA {X—R⁵=NH₂} in an aqueous solution of acid, for example in hydrochloric acid, hydrobromic acid or sulfuric acid, with a nitrosating agent, for example a nitrite, such as sodium nitrite and potassium nitrite.

For preparing the diazonium salt IA {X—R⁵=N₂⁺}, the amino compound IA {X—R⁵=NH₂} can be reacted in the absence of water, for example in glacial acetic acid containing hydrogen chloride, in absolute alcohol, in dioxane or tetrahydrofuran, in acetonitrile or in acetone, with a nitrite, such as tert-butyl nitrite and isopentyl nitrite.

The conversion of the resulting diazonium salt into the corresponding compound IA where X—R⁵=cyano, chlorine, bromine or iodine is particularly preferably carried out by treatment with a solution or suspension of a copper(I) salt, such as copper(I) cyanide, chloride, bromide or iodide, or with a solution of an alkali metal salt (cf. A1).

The conversion of the resulting diazonium salt into the corresponding hydroxyl compound IA {X—R⁵=hydroxyl} is advantageously carried out by treatment of the diazonium salt IA with an aqueous acid, preferably sulfuric acid. The addition of a copper(II) salt, such as copper(II) sulfate, can have an advantageous effect on the course of the reaction. In general, this reaction is carried out at from 0 to 100° C., preferably at the boiling point of the reaction mixture.

Compounds IA where X—R⁵=mercapto, C₁-C₆-alkylthio or halosulfonyl are obtained, for example, by reacting the diazonium salt in question of IA with hydrogen sulfide, an alkali metal sulfide, a dialkyl disulfide, such as dimethyl disulfide, or with sulfur dioxide.

The Meerwein arylation usually entails reacting the diazonium salts with alkenes or alkines. The alkene or alkine is preferably employed in an excess of up to about 3000 mol %, based on the amount of the diazonium salt.

The reactions described above of the diazonium salt IA {X—R⁵=N₂⁺} can be carried out, for example, in water, in aqueous hydrochloric acid or hydrobromic acid, in a ketone, such as acetone, diethyl ketone and methyl ethyl ketone, in a nitrile, such as acetonitrile, in an ether, such as dioxane and tetrahydrofuran, or in an alcohol, such as methanol and ethanol.

Unless stated otherwise for the individual reactions, the reaction temperatures are usually from −30° C. to 50° C.

All reaction partners are preferably employed in approximately stoichiometric amounts; however, an excess of one or the other component of up to about 3000 mol % may also be advantageous.

The mercapto compounds IA {X—R⁵=SH} can also be obtained by reducing the compounds IA described below where X—R⁵=halosulfonyl. Suitable reducing agents are, for example, transition metals, such as iron, zinc and tin (cf., for example, “The Chemistry of the Thiol Group”, John Wiley, 1974, p. 216).

C.2 Halosulfonation of 3-arylisothiazoles IA in which XR⁵ is hydrogen:

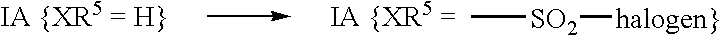

The halosulfonation can be carried out in the absence of a solvent in an excess of sulfonating agent, or in an inert solvent/diluent, for example in a halogenated hydrocarbon, an ether, an alkylnitrile or a mineral acid.

Chlorosulfonic acid is the preferred agent as well as the preferred solvent.

The amount of sulfonating agent used is usually slightly less than (up to about 95 mol %) or an excess of 1 to 5 times the molar amount of the starting material IA (where X—R⁵=H). In the absence of an inert solvent, it may also be advantageous to employ an even larger excess.

The reaction temperature is usually from 0° C. to the boiling point of the reaction mixture.

For work-up, the reaction mixture is mixed, for example, with water, whereupon the product can be isolated as usual.

C.3 Side-chain halogenation of 3-arylisothiazoles IA in which X—R⁵ is methyl, and conversion of the products into further compounds of the formula IA:

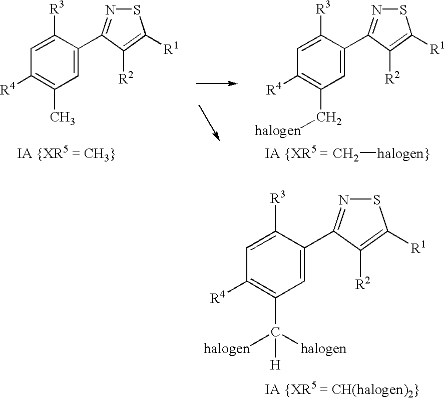

Examples of suitable solvents include organic acids, inorganic acids, aliphatic or aromatic hydrocarbons, which may be halogenated, and also ethers, sulfides, sulfoxides and sulfones.

Suitable halogenating agents are, for example, chlorine, bromine, N-bromosuccinimide, N-chlorosuccinimide or sulfuryl chloride. Depending on the starting material and the halogenating agent used, the addition of a free-radical initiator, for example an organic peroxide, such as dibenzoyl peroxide, or an azo compound, such as azobisisobutyronitrile, or irradiation with light, may have an advantageous effect on the course of the reaction.

The amount of halogenating agent is not critical. Both substoichiometric amounts and large excesses of halogenating agent, based on the compound IA to be halogenated (where X—R⁵=methyl), are possible.

When using a free-radical initiator, a catalytic amount is usually sufficient.

The reaction temperature is usually from −100° C. to 200° C., mainly from 10 to 100° C. or the boiling point of the reaction mixture.

By a nucleophilic substitution, those halogenation products IA where X—R⁵=CH₂-halogen can be converted according to the scheme below into their corresponding ethers, thioethers, esters, amines or hydroxylamines:

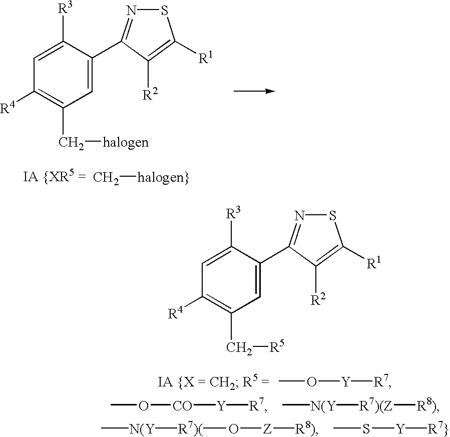

The nucleophile used is either a suitable alcohol, thiol, carboxylic acid or amine, the reaction in this case being preferably carried out in the presence of a base (for example an alkali metal hydroxide or an alkaline earth metal hydroxide or an alkali metal carbonate or an alkaline earth metal carbonate), or the alkali metal salts of these compounds obtained by reaction of the alcohol, thiol, carboxylic acid or amine with a base (for example an alkali metal hydride).

Particularly suitable solvents are aprotic organic solvents, for example tetrahydrofuran, dimethyl formamide, dimethyl sulfoxide, or hydrocarbons, such as toluene and n-hexane.

The reaction is carried out at a temperature from the melting point to the boiling point of the reaction mixture, preferably at from 0 to 100° C.

Those halogenation products IA where X—R⁵=CH(halogen)₂ can be hydrolyzed give to the corresponding aldehydes (IA where X—R⁵=CHO). The latter can in turn be oxidized analogously to known processes to give the carboxylic acids IA {X—R⁵=COOH}:

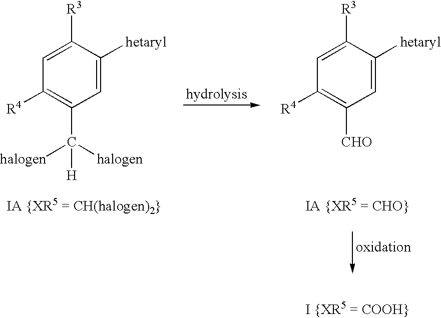

The hydrolysis of the compounds IA where X—R⁵=dihalomethyl is preferably carried out under acidic conditions, in particular without a solvent in hydrochloric acid, acetic acid, formic acid or sulfuric acid, or in an aqueous solution of one of the acids mentioned, for example in a mixture of acetic acid and water (for example 3:1).

The reaction temperature is usually at from 0 to 120° C.

The oxidation of the hydrolysis products IA where XR⁵=formyl to give the corresponding carboxylic acids can be carried out in a manner known per se, for example according to Kornblum (see in particular pages 179 to 181 of the volume “Methods for the Oxidation of Organic Compounds” by A. H. Haines, Academic Press 1988, in the series “Best Synthetic Methods”). A suitable solvent is, for example, dimethyl sulfoxide.

The aldehydes IA {X—R⁵=CHO} can also be converted in a manner known per se into olefinic compounds IA where X=unsubstituted or substituted ethene-1,2-diyl:

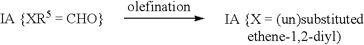

The olefination is preferably carried out by the method of Wittig or one of its modifications, suitable reaction partners being phosphorus ylides, phosphonium salts and phosphonates, or by aldol condensation.

If a phosphonium salt or a phosphonate is used, it is advantageous to carry out the reaction in the presence of a base, particularly suitable bases being alkali metal alkyls, such as n-butyllithium, alkali metal hydrides and alkoxides, such as sodium hydride, sodium ethoxide and potassium tert-butoxide, and alkali metal hydroxides and alkaline earth metal hydroxides, such as calcium hydroxide.

For a complete conversion, all reaction partners are employed in a ratio which is about stoichiometric; however, preference is given to using an excess of phosphorus compound and/or base of up to about 10 mol %, based on the starting material (IA where X—R⁵=CHO).

The reaction temperature is generally from −40 to 150° C.

The 3-arylisothiazoles IA where X—R⁵=formyl can be converted in a manner known per se into the compounds IA where X—R⁵=—CO—Y—R⁷, for example by reaction with a suitable organometal compound Me—Y—R⁷—where Me is a base metal, preferably lithium or magnesium—and subsequent oxidation of the alcohols obtained in this reaction (cf., for example, J. March, Advanced Organic Chemistry, 3rd ed., John Wiley, New York 1985, pp. 816ff. and 1057ff.).

The compounds IA where X—R⁵=—CO—Y—R⁷ can in turn be reacted further in a Wittig reaction. The phosphonium salts, phosphonates or phosphorus ylides required as reaction partners are already known or can be prepared in a manner known per se (cf., for example, Houben-Weyl, Methoden der organischen Chemie, Vol. E1 , p. 636ff. and Vol. E2, p. 345ff., Georg Thieme Verlag Stuttgart 1982; Chem. Ber. 95 (1962), 3993}.

Further possible ways of preparing other 3-arylisothiazoles IA from compounds IA where X—R⁵=formyl include the aldol condensation known per se, and condensation reactions according to Knoevenagel or Perkin. Suitable conditions for these processes are described, for example, in Nielson, Org. React. 16, (1968), 1ff. {aldol condensation} Org. React. 15, (1967), 204ff. {Knoevenagel condensation} and Johnson, Org. React. 1, (1942), 210ff. {Perkin condensation}.

The compounds IA where X—R⁵=—CO—Y—R⁷ can also be converted in a manner known per se into their corresponding oximes {cf., for example, Houben-Weyl, Methoden der Organischen Chemie, Georg Thieme Verlag Stuttgart, Vol. 10/4, 4th edition 1968, p. 55ff. and p. 73ff.}:

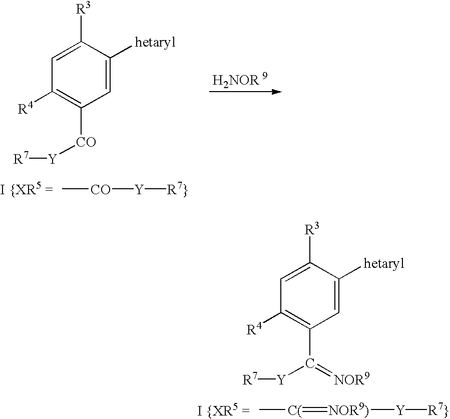

C.4 Synthesis of ethers, thioethers, amines, esters, amides, sulfonamides, thioesters, hydroximic esters, hydroxylamines, sulfonic acid derivatives, oximes or carboxylic acid derivatives:

3-Arylisothiazoles IA where R⁵ is hydroxyl, amino, —NH—Y—R⁷, hydroxylamino, —N(Y—R⁷)—OH, —NH—O—Y—R⁷, mercapto, halosulfonyl, —C(═NOH)—Y—R⁷, carboxyl or —CO—NH—O—Z—R⁸ can be converted in a manner known per se by alkylation, acylation, sulfonation, esterification or amidation into the corresponding ethers {IA where R⁵=—O—Y—R⁷}, esters {I where R⁵=—O—CO—Y—R⁷}, amines {I where R⁵=—N(Y—R⁷)(Z—R⁸)}, amides {IA where R⁵=—N(Y—R⁷)—CO—Z—R⁸}, sulfonamides {IA where R⁵=—N(Y—R⁷)—SO₂—Z—R⁸ or —N(SO₂—Y—R⁷)(SO₂—Z—R⁸)}, hydroxylamines {IA where R⁵=—N(Y—R⁷)(O—Z—R⁸)}, thioethers {IA where R⁵=—S—Y—R⁷}, sulfonic acid derivatives {IA where R⁵=—SO₂—Y—R⁷, —SO₂—O—Y—R⁷ or —SO₂—N(Y—R⁷)(Z—R⁸)}, oximes (IA where R⁵=—C(═NOR⁹)—Y—R⁷}, carboxylic acid derivatives {IA where R⁵=—CO—O—Y—R⁷, —CO—S—Y—R⁷, —CO—N(Y—R⁷)(Z—R⁸), —CO—N(Y—R⁷)(O—Z—R⁸)} or hydroximic esters {I where R⁵=—C(═NOR⁹)—O—Y—R⁷}.

Such conversions are described, for example, in Houben-Weyl, Methoden der Organischen Chemie, Georg Thieme Verlag Stuttgart (Vol. E16d, p. 1241ff.; Vol. 6/1a, 4th edition 1980, p. 262ff.; Vol. 8, 4th edition 1952, p. 471ff., 516ff., 655ff. and p. 686ff.; Vol. 6/3, 4th edition 1965, p. 10ff.; Vol. 9, 4th edition 1955, p. 103ff., 227ff., 343ff., 530ff., 659ff., 745ff. and p. 753ff.; Vol. E5, p. 934ff., 941ff. and p. 1148ff.).

Ethers (compounds I where X—R⁵=O—Y—R⁷) can be prepared in good yields, for example, by reacting the corresponding hydroxyl compound (compound I where X—R⁵=OH) with an aliphatic halide Hal-Y—R⁷ (Hal=chlorine, bromine or iodine). The reaction is carried out in the manner described for the alkylation of phenols (for the ether synthesis, see, for example, J. March “Advanced Organic Chemistry” 3rd ed. p. 342 f. and literature cited therein), preferably in the presence of a base such as NaOH or an alkali metal carbonate or sodium hydride. Preferred reaction media are aprotic polar solvents, such as dimethylformamide, N-methylpyrrolidone or dimethylacetonitrile.

D) Preparation of compounds of the formula I in which Q is nitrogen (compounds IB).

In addition to the processes already mentioned in sections A, B and C above, the processes D.1 and D.2 below are particularly suitable:

D.1 Halogenation of the pyridine ring of compounds IB where X—R⁵=H: to this end, a 3-pyridylisothiazole of the formula IB (X—R⁵=H) is preferably initially converted into the corresponding pyridine N-oxide of the formula IX. In formula IX, R¹, R², R³ and R⁴ are as defined above.

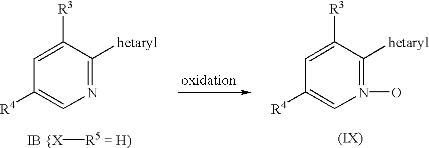

Suitable oxidizing agents for this reaction are, for example, hydrogen peroxide or organic peracids, for example performic acid, peracetic acid, trifluoroperacetic acid or m-chloroperbenzoic acid.

Suitable solvents are organic solvents which are inert to oxidation, such as, for example, hydrocarbons, such as toluene or hexane, ethers, such as diethyl ether, dimethoxyethane, methyl t-butyl ether, dioxane or tetrahydrofuran, alcohols, such as methanol or ethanol, or else mixtures of such solvents with one another or with water. If the oxidation is carried out using an organic peracid, the preferred solvent is the parent organic acid, i.e., for example, formic, acetic or trifluoroacetic acid, if appropriate in a mixture with one or more of the abovementioned solvents.

The reaction temperature is usually from the melting point to the boiling point of the reaction mixture, preferably at 0-150° C.

To obtain a high yield, it is frequently advantageous to employ the oxidizing agent in a molar excess of up to about five times, based on the IB (where X—R⁵=H) used.

The pyridine N-oxide IX is then converted into IB (X—R⁵=halogen) by reaction with a halogenating agent.

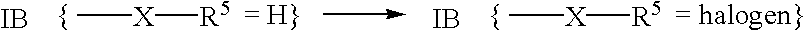

Suitable halogenating agents are phosphoryl halides, such as POCl₃ or POBr₃, phosphorus halides, such as PCl₅, PBr₅, PCl₃ or PBr₃, phosgene or organic or inorganic acid halides, such as, for example, trifluoromethanesulfonyl chloride, acetyl chloride, bromoacetyl bromide, acetyl bromide, benzoyl chloride, benzoyl bromide, phthaloyl dichloride, toluenesulfonyl chloride, thionyl chloride or sulfuryl chloride. If appropriate, it may be advantageous to carry out the reaction in the presence of a base, such as, for example, trimethylamine or triethylamine or hexamethyldisilazane.

Suitable solvents are inert organic solvents, such as, for example, hydrocarbons, such as toluene or hexane, ethers, such as diethyl ether, dimethoxyethane, methyl t-butyl ether, dioxane or tetrahydrofuran, amides, such as DMF, DMA or NMP, or mixtures thereof. If the reaction is carried out using a liquid halogenating agent, this can preferably also be used as solvent, if appropriate in a mixture with one of the abovementioned solvents.

The reaction temperature is usually from the melting point to the boiling point of the reaction mixture, preferably at 50-150° C.

To obtain a high yield, it may be advantageous to employ a molar excess of halogenating agent or base of up to about five times, based on the IX used.

D.2 Nucleophilic substitution on halopyridines of the formula IB (X—R⁵=halogen). In the scheme below, examples of the classes of compounds obtainable by this route are shown.

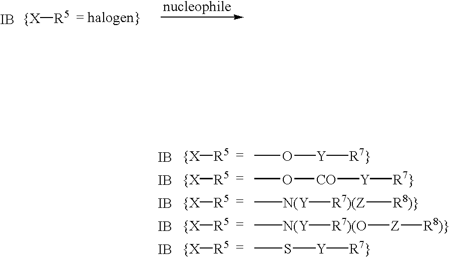

Suitable nucleophiles are alcohols, thiols, amines, carboxylic acids or CH-acidic compounds, for example nitroalkanes, such as nitromethane, malonic acid derivatives, such as diethyl malonate, or cyanoacetic acid derivatives, such as methyl cyanoacetate. To carry out this reaction, what has been said under C.3 applies.

E) Preparation of compounds of the formula I in which R⁴ together with X—R⁵ or R⁶ together with X—R⁵ is one of the chains —N═C(R¹⁸)—S— (compounds IC-1 or compounds ID-1) or —N═C(R¹⁸)—O— (compounds IC-2 and compounds ID-2).

For the preparation of the compounds IC and ID, it is likewise possible to use the processes mentioned in sections A and B, or to use these processes for preparing suitable starting materials.

Furthermore, the compounds IC-1, IC-2, ID-1 and ID-2 can be synthesized analogously to known processes by ring closure reactions from the corresponding ortho-aminophenols or ortho-mercaptoanilines of the formulae IA-1, IA-2, IA-3 or IA-4; numerous methods for this purpose are known from the literature (see, for example, Houben-Weyl, Methoden der Organischen Chemie, Vol. E8a, p. 1028ff., Georg-Thieme-Verlag, Stuttgart 1993 and Vol. E8b, p. 881ff., Georg-Thieme-Verlag, Stuttgart 1994). In the formulae IA-1 to IA-4, the variables R¹, R², R³ and R⁴ are as defined above. The variables X¹, X², X³ and X⁴ are, independently of one another, OH or SH.

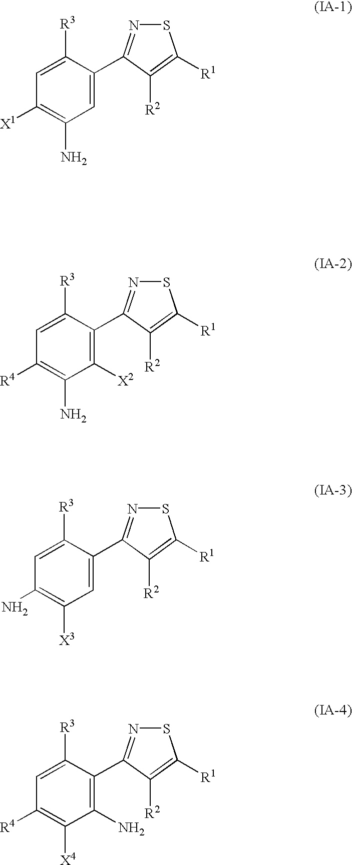

E.1 Compounds IC-1 or ID-1, in which R⁴ together with X—R⁵ or R⁶ together with X—R⁵ forms one of the chains —N═C(R¹⁸)—S— can also be prepared, in particular, by the process shown below:

This process entails the reaction of an aminophenylisothiazole of the formula IA-5, IA-6, IA-7 or IA-8 with halogen and ammonium thiocyanate or with an alkali metal or alkaline earth metal thiocyanate. This gives compounds of the formulae IC-1a, IC-1b or ID-1a or ID-1b respectively (compounds IC-1 or ID-1 in which R¹⁸ is NH₂).

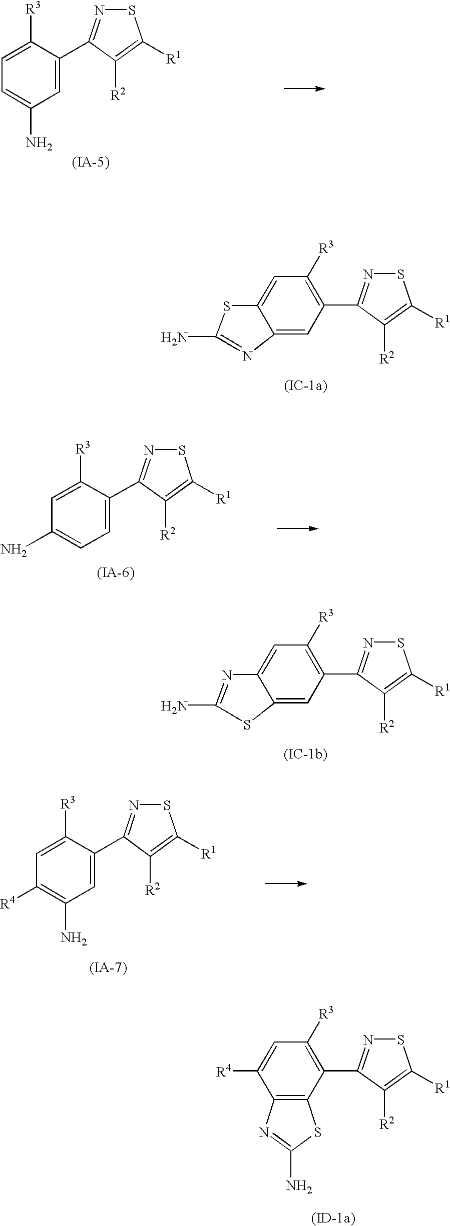

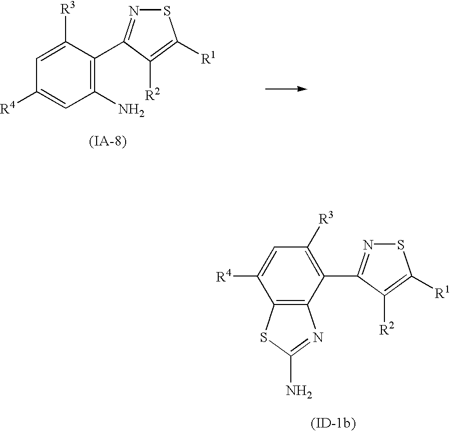

By subsequent reactions at the amino group, these compounds can be converted into other compounds IC-1 or ID-1.

Preferred halogen is chlorine or bromine; among the alkali metal/alkaline earth metal thiocyanates, preference is given to sodium thiocyanate.

In general, the reaction is carried out in an inert solvent/diluent, for example in a hydrocarbon, such as toluene and hexane, in a halogenated hydrocarbon, such as dichloromethane, in an ether, such as tetrahydrofuran, in an alcohol, such as ethanol, in a carboxylic acid, such as acetic acid, or in a polar aprotic solvent/diluent, such as dimethyl formamide, acetonitrile and dimethyl sulfoxide.

The reaction temperature is usually from the melting point to the boiling point of the reaction mixture, preferably at from 0 to 150° C.

To obtain a high yield of the product of value, the halogen and ammonium thiocyanate or alkali metal/alkaline earth metal thiocyanate are preferably employed in an about equimolar amount or in an excess of up to about 5 times the molar amount, based on the amount of IA-5, IA-6, IA-7 or IA-8.

A variant of the process comprises converting the NH₂ group of the aminophenylisothiazoles IA-5, IA-6, IA-7 or IA-8 initially with ammonium thiocyanate or an alkali metal or alkaline earth metal thiocyanate into a thiourea group (NH—C(S)—NH₂ group), which is then converted by treatment with a halogen into the benzothiazoles (compounds IC-1 or ID-1 where R¹⁸=NH₂).

Finally, it is possible to carry out reactions analogous to those which have already been described in section C.1) at the amino group of the chain —N═C(NH₂)—S—.

E.2 Compounds of the formulae IC and ID in which R⁴ together with X—R⁵ or R⁶ together with X—R⁵ form one of the chains —N═C(R¹⁸)—O— can be prepared by conversion of the NH₂ group in the aminophenylisothiazoles of the formula IA-5, IA-6, IA-7 or IA-8 into an azide group (N₃ group) and subsequent cyclization of the resulting azidophenylisothiazoles with a carboxylic acid to give compounds of the formula IC-2a, IC-2b, ID-2a or ID-2b.

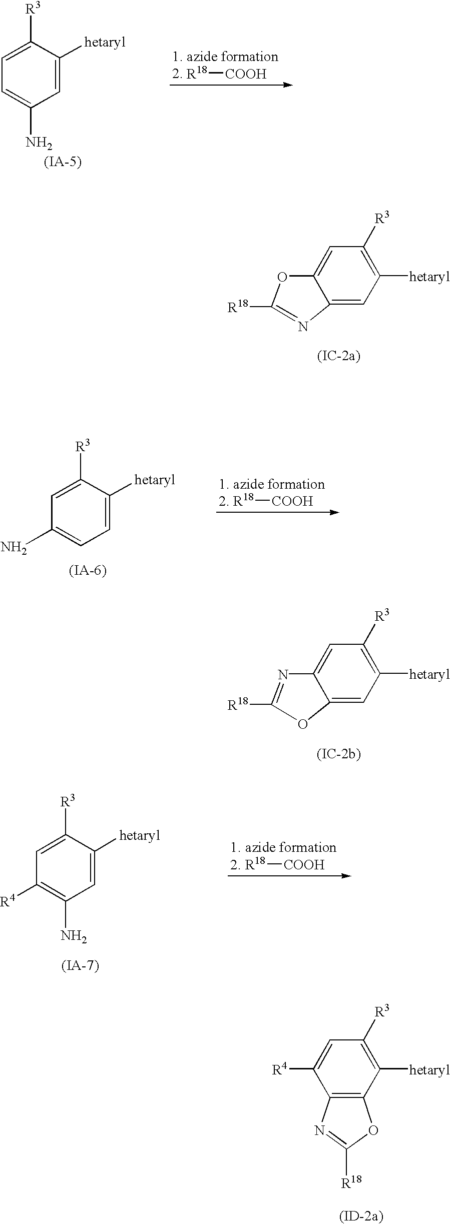

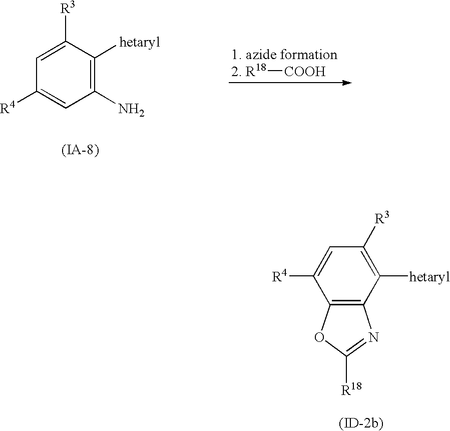

The conversion of the amino group in the aminophenylisothiazoles of the formula IA-5, IA-6, IA-7 or IA-8 into an azide group is generally carried out in two steps, i.e. by diazotization of the amino group and subsequent treatment of the resulting diazonium salt with an azide. To carry out the diazotization, what has been stated in process C.1) applies. The conversion into the aryl azides is preferably carried out by reacting diazonium salts with an alkali metal or alkaline earth metal azide, such as sodium azide, or by reaction with trimethylsilyl azide.

The reaction of the azide compounds IA (X—R⁵=N₃) with the carboxylic acid R¹⁸—COOH is carried out either in an inert organic solvent, for example in hydrocarbons, such as toluene or hexane, in halogenated hydrocarbons, such as dichloromethane or chloroform, in ethers, such as diethyl ether, dimethoxyethane, methyl t-butyl ether, dioxane or tetrahydrofuran, in amides, such as DMF, DMA or NMP, in acetonitrile or, preferably, in the absence of solvent in an excess of the carboxylic acid R¹⁸COOH. In the latter case, it may be helpful to add a mineral acid, such as phosphoric acid, or a silylating agent, such as a mixture of phosphorus pentoxide and hexamethyldisiloxane.

The reaction is preferably carried out at elevated temperature, for example at the boiling point of the mixture.

F) Compounds of the formula I in which X—R⁵ together with R⁴ or R⁶ forms one of the chains —O—C(R¹⁵,R¹⁶)—CO—N(R¹⁷)— or —S—C(R^{15,}R¹⁶)—CO—N(R¹⁷)— can be prepared by the processes mentioned in sections A and B. Moreover, they can be prepared in principle from the corresponding aminophenols or mercaptoanilines IA-1, IA-2, IA-3 or IA-4 by known processes, for example by the process described in U.S. Pat. No. 4,798,620. With a view to this reaction, the disclosure of this publication is expressly incorporated herein by way of reference.

In particular, those compounds of the formula I in which X—R⁵ together with R⁴ or together with R⁶ form a chain —O—C(R¹⁵,R¹⁶)—CO—N(R¹⁷)— can also be prepared from nitrophenoxyacetic acid derivatives of the formulae IA-9, IA-10, IA-11 and IA-12. The conversion is carried out by reducing the nitro groups in IA-9, IA-10, IA-11 or IA-12, where generally simultaneously with the reduction ring closure takes place, to give the compounds of the formula IC-3a, IC-3b, ID-3a or ID-3b.

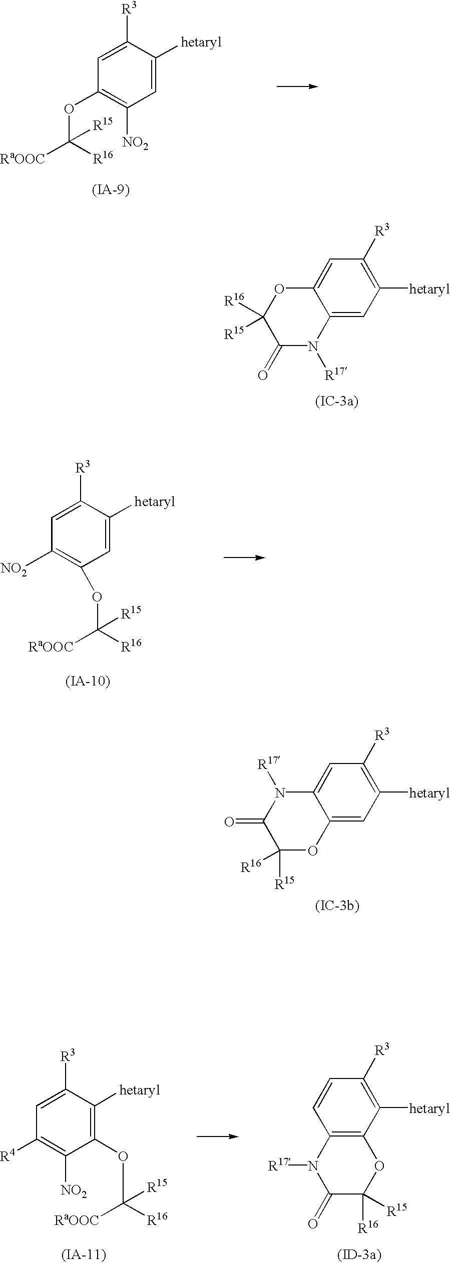

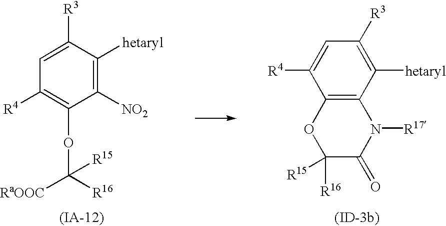

In the formulae IA-9, IA-10, IA-11, IA-12, IC-3a, IC-3b, ID-3a or ID-3b, R¹, R², R³, R⁴, R¹⁵ and R¹⁶ are as defined above. R^{17′} is H or OH. R^{a} is a nucleophilically displaceable leaving group, for example a C₁-C₄-alkyl radical, such as methyl or ethyl.

These reductions can be carried out under the conditions mentioned in section C.1) for the reduction of aromatic nitro groups.

If desired, the reaction products can be converted by alkylation into further compounds of the formula IC-3 or ID-3. For carrying out these reactions, what has been said under section C.4 applies correspondingly.

If not stated otherwise, all the processes described above are advantageously carried out under atmospheric pressure or under the autogenous vapor pressure of the reaction mixture in question.

The preparation of the 7-(isothiazolyl)-1,3-benzoxazoles of the formula I-D according to the invention

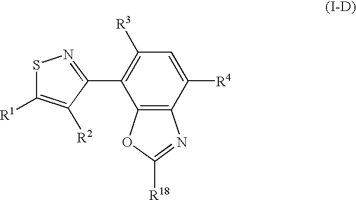

is furthermore surprisingly possible by cyclizing a 2-halo-3-(isothiazol-3-yl)anilide of the formula X,

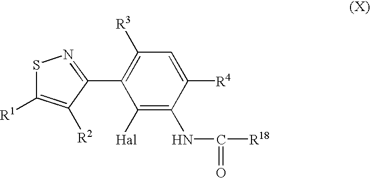

in the presence of a transition metal compound of transition groups VIIa, VIIIa or Ib of the Periodic Table and a base, where the variables R¹ to R⁴ and R¹⁸ in formula X have the meanings mentioned above and Hal is bromine or iodine.

Suitable transition metal compounds are, for example, manganese, rhenium, iron, ruthenium, osmium, cobalt, rhodium, iridium, nickel, palladium, platinum, copper, silver or gold compounds, in particular copper, manganese, palladium, cobalt or nickel compounds. Examples of compounds of the abovementioned transition metals are their halides, such as MnCl₂, MnBr₂, MnI₂, ReCl₃, ReBr₃, ReI₃, ReCl₄, ReBr₄, ReI₄, ReCl₅, ReBr₅, ReCl₆, FeCl₂, FeBr₂, FeI₂, FeCl₃, FeBr₃, RuCl₂, RuBr₂, RuI₂, RuCl₃, RuBr₃, RuI₃, OsI, OsI₂, OsCl₃, OsBr₃, OSI₃, OsCl₄, OsBr₄, OsCl₅, COCl₂, CoBr₂, CoI₂, RhCl₃, RhBr₃, RhI₃, IrCl₃, IrBr₃, IrI₃, NiCl₂, NiBr₂, NiI₂, PdCl₂, PdBr₂, PdI₂, PtCl₂, PtBr₂, PtI₂, PtCl₃, PtBr₃, PtI₃, PtCl₄, PtBr₄, PtI₄, CuCl, CuBr, CuI, CuCl₂, CuBr₂, AgCl, AgBr, AgI, AuCl, AuI, AuCl₃, AuBr₃ and also their oxides and sulfides, for example Cu₂S and Cu₂O. It is also possible to employ the transition metal in question as such for the process according to the invention if it is transformed under reaction conditions into the actual catalytically active transition metal compound.

In a preferred embodiment of the process according to the invention, the transition metal used is a copper(II) and/or a copper(I) compound, in particular a copper(I) halide, for example copper(I) chloride, copper(I) bromide or copper(I) iodide.

In addition to the transition metal compound which catalyzes the cyclization of X into I-D, it is also possible to use, in the process according to the invention, a cocatalyst which is a compound which constitutes a complex ligand for the transition metal in question. Examples of cocatalysts are phosphines, such as triphenylphosphine, tri-o-tolylphosphine, tri-n-butyl-phosphine, 1,2-bis(diphenylphosphino)ethane, 1,3-bis(diphenyl-phosphino)propane, phosphites, such as trimethyl phosphite, triethyl phosphite or triisopropyl phosphite, sulfides, such as dimethyl sulfide, and also cyanide or carbon monoxide. If desired, the cocatalyst is generally employed in an at least equimolar amount, based on the transition metal.

It is also possible to employ the transition metal compounds as complex compounds which, preferably, carry one or more of the abovementioned cocatalysts as ligands. Examples of such compounds are [NiCl₂(PPh₃)₂], [Pd(PPh₃)₄], [PdCl₂(PPh₃)₂], [PdCl₂(dppe)], [PdCl₂(dppp)], [PdCl₂(dppb)], [CuBr(S(CH₃)₂)], [CuI(P(OC₂H₅)₃)], [CuI(P(OCH₃)₃)], [CuCl(PPh₃)₃] or [AuCl(P(OC₂H₅)₃)].

If desired, the transition metal compounds can also be immobilized on an inert support, for example on activated carbon, silica gel, alumina or on an insoluble polymer, for example a styrene-divinylbenzene copolymer.

In the process according to the invention, the transition metal compounds can be employed both in an equimolar amount, based on the compound X, and in a substoichiometric amount or in excess. The molar ratio of transition metal to the compound X used is usually in the range from 0.01:1 to 5:1, preferably in the range from 0.02:1 to 2:1, and in particular in the range from 0.05:1 to about 1:1.5. In a preferred variant, an equimolar amount of transition metal compound is used, i.e. the molar ratio of transition metal to the compound X used is about 1:1. However, the transition metal compound is particularly preferably employed in a catalytic, i.e. substoichiometric, amount. In this case, the molar ratio of transition metal to the compound X used is <1:1. In this variant, the molar ratio of transition metal compound to the compound X used is particularly preferably in the range from 0.05:1 to 0.8:1, for example from 0.1:1 to 0.3:1.

According to the invention, the process is carried out in the presence of a base. Suitable bases are, in principle, all basic compounds capable of deprotonating the amide group in X. Preference is given to bases such as alkoxides, amides, hydrides, hydroxides, bicarbonates and carbonates of alkali metals or alkaline earth metals, in particular of lithium, potassium, sodium, cesium or calcium. Examples of suitable bases are the sodium alkoxides or potassium alkoxides of methanol, of ethanol, of n-propanol, of isopropanol, of n-butanol and of tert-butanol, furthermore sodium hydride and potassium hydride, calcium hydride, sodium amide, potassium amide, sodium carbonate, potassium carbonate, cesium carbonate, sodium bicarbonate, potassium bicarbonate, sodium hydroxide, potassium hydroxide and lithium hydroxide. In a preferred embodiment of the process, the base used is sodium hydride. In another, particularly preferred embodiment of the process, the base used is potassium carbonate and/or potassium bicarbonate. The base can be employed in a substoichiometric or equimolar amount, or in excess. Preferably, at least an equimolar amount of base, based on the compound X, is used. In particular, the molar ratio of base (calculated as base equivalents) to the compound X is in the range from 1:1 bis 1:5 and particularly preferably in the range from 1:1 to 1:1.5.

The conversion of X into I-D is preferably carried out in an organic solvent. Suitable solvents are, in principle, all organic solvents which are inert under the reaction conditions. These are, for example, hydrocarbons, such as hexane or toluene, halogenated hydrocarbons, such as 1,2-dichloroethane or chlorobenzene, ethers, such as dioxane, tetrahydrofuran (THF), methyl tert-butyl ether, dimethoxyethane, diethylene glycol dimethyl ether and triethylene glycol dimethyl ether, aprotic polar solvents, for example organic amides, such as dimethylformamide (DMF), N-methylpyrrolidone (NMP), N,N-dimethylacetamide (DMA), dimethyl sulfoxide (DMSO), organic nitriles, such as acetonitrile or propionitrile, and also tertiary nitrogen bases, for example pyridine. It is, of course, also possible to use mixtures of the solvents mentioned. Preference is given to aprotic polar solvents, such as DMSO, DMF, NMP, DMA, acetonitrile, propionitrile, pyridine, dimethoxyethane, diethylene glycol dimethyl ether and triethylene glycol dimethyl ether, or mixtures of these.

Naturally, the reaction temperature depends on the reactivity of the compound X in question. In general, the reaction temperature will not be below room temperature. Preferably, the conversion of X into I-D is carried out at temperatures below 200° C. Frequently, the reaction will be carried out at elevated temperature, for example above 50° C., in particular above 70° C. and particularly preferably above 100° C. The reaction is preferably carried out at temperatures below 180° C. and in particular below 160° C.

Work-up of the reaction product to yield the target compound I-D can be carried out using the methods customary for this purpose. In general, work-up will initially be by extraction, or the solvent used is removed by customary methods, for example by distillation. It is also possible, after dilution of the reaction mixture with water, to extract the target compound I-D from the reaction mixture using a volatile organic solvent which for its part is removed by distillation. It is also possible to precipitate the target compound from the reaction mixture by adding water. This gives a crude product which contains the product of value I-D. For further purification, customary methods such as crystallization or chromatography, for example on alumina or silica gel, may be employed. To obtain the pure isomers, it is also possible to chromatograph the substances obtainable by the process on optically active adsorbates.

For the cyclization of X to I-D, preference is given to using compounds X in which R² in formula X is preferably a radical different from hydrogen. Preference is given to using those compounds of the formula X in which the variables R¹ to R⁴ and R¹⁸ independently of one another, but preferably in combination with one another, are as defined below:

R¹ is C₁-C₄-haloalkyl, C₁-C₄-haloalkoxy, C₁-C₄-alkylsulfonyl, or alkylsulfonyloxy, in particular trifluoromethyl, difluoromethoxy, methylsulfonyl or methylsulfonyloxy;

R² is halogen, cyano, C₁-C₄-alkyl; specifically chlorine;

R³ is hydrogen or halogen; in particular fluorine or chlorine;

R⁴ is fluorine, chlorine or cyano;

R¹⁸ is hydrogen, C₁-C₄-alkyl, C₁-C₄-haloalkyl, C₂-C₄-alkenyl, C₂-C₄-haloalkenyl, C₂-C₄-alkinyl, C₁-C₄-alkoxy-C₁-C₄-alkyl, C₁-C₄-alkoxycarbonyl-C₁-C₄-alkyl, C₃-C₈-cycloalkyl, C₃-C₈-cycloalkyl-C₁-C₄-alkyl, phenyl, phenyl-C₁-C₄-alkyl, 4- to 7-membered heterocyclyl, where the phenyl ring, the cycloalkyl ring and the heterocyclyl ring may be unsubstituted or may carry one or two substituents selected from the group consisting of cyano, halogen, C₁-C₄-alkyl, C₁-C₄-haloalkyl and C₁-C₄-alkoxy.

R¹⁸ is in particular hydrogen, C₁-C₄-alkyl, C₁-C₄-alkoxy-C₁-C₄-alkyl, C₃-C₈-cycloalkyl, C₃-C₈-cycloalkyl-C₁-C₄-alkyl, phenyl or phenyl-C₁-C₄-alkyl.

The compounds of the formula X are novel and are useful intermediates for the preparation of benzoxazoles of the formula I-D. Accordingly, the compounds of the formula X also form part of the subject matter of the present invention.

Surprisingly, it has been found that the compounds of the formula X can be prepared in good yields from the 3-(isothiazol-3-yl)anilines of the formula IA (XR⁵=NH₂) described further above:

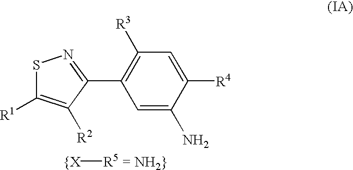

The process for preparing the compounds X from the compounds IA comprises the following process steps:

i. halogenation of a 3-(isothiazol-3-yl)aniline of the formula IA (XR⁵=NH₂) to give a 2-halo-3-(isothiazol-3-yl)-aniline of the formula XI,

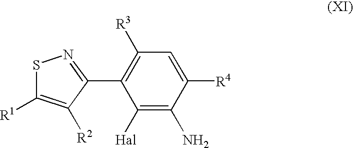

ii. reaction of the 2-halo-3-(isothiazol-3-yl)aniline XI with an acylating agent of the formula R¹⁸—C(O)—L where L is a leaving group, to give an anilide of the formula X and/or a diacyl compound of the formula XII,

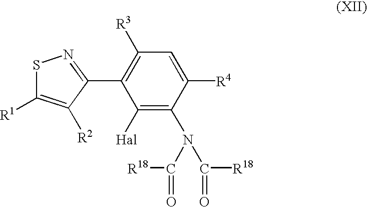

iii. if appropriate, partial solvolysis of the compound XII to give the anilide of the formula X,

where in the compounds of the formulae IA, XI and XII, the variables R¹-R⁴, R¹⁸ and Hal are as defined above. With respect to preferred and particularly preferred meanings of these variables, what was said above with respect to the compounds X applies. This variant is used in particular in cases where R² is different from hydrogen.

The 3-(isothiazol-3-yl)anilines of the formula IA (XR⁵=NH₂) used as starting materials can be obtained by the reaction sequence described above.

The 2-halo-3-(isothiazol-3-yl)anilines of the formula XI and the N,N-diacyl-2-halo-3-(isothiazol-3-yl)anilines of the formula XII are likewise novel and are useful intermediates for the preparation of I-D from X.

Suitable halogenating agents for converting compounds of the formula IA (XR⁵=NH₂) into the 2-halo-3-(isothiazol-3-yl)-anilines of the formula XI (step i)) are bromine, mixtures of chlorine and bromine, bromine chloride, iodine, mixtures of iodine and chlorine, iodine chloride, N-halosuccinimides, such as N-bromosuccinimide, N-iodosuccinimide, hypohalic acids, such as hypobromic acid, furthermore dibromoisocyanuric acid and the bromine/dioxane complex. The halogenating agent is generally employed in an equimolar amount or in excess, based on IA (XR⁵=NH₂), preferably approximately in the stoichiometrically required amount. The molar excess can be up to 5 times the amount of IA (XR⁵=NH₂). From among the abovementioned halogenating agents, preference is given to the brominating agents and the iodinating agents, where, in a preferred embodiment of the process, elemental bromine is used.

If appropriate, it is possible to use catalytic or stoichiometric amounts of a Lewis- or Brönsted-acidic catalyst, for example aluminum chloride or aluminum bromide, iron(III) chloride or iron(III) bromide, or sulfuric acid, or a catalyst precursor which forms the actual catalyst during the reaction, for example iron, can be used to accelerate the reaction i). If the compound XI is to be prepared as an iodide (Hal=iodine), it is also possible to use, as catalyst, nitric acid, iodic acid, sulfur trioxide, hydrogen peroxide or an aluminum chloride/copper(II) chloride complex.

In another variant of the reaction i), the desired halogen is employed in the form of a halide salt which, on addition of an oxidizing agent, releases the halogen. Examples of such “halogenating agents” are mixtures of sodium chloride or sodium bromide and hydrogen peroxide.

The halogenation is usually carried out in an inert solvent, for example a hydrocarbon, such as hexane, a halogenated hydrocarbon, such as dichloromethane, trichloromethane, 1,2-dichloroethane or chlorobenzene, in a cyclic ether, such as dioxane, in a carboxylic acid, such as acetic acid, propionic acid or butanoic acid, in a mineral acid, such as hydrochloric acid or sulfuric acid, or in water. It is, of course, also possible to use mixtures of the solvents mentioned above.

If appropriate, the reaction is carried out in the presence of a base, for example an alkali metal hydroxide, such as KOH, or the alkali metal salt of a carboxylic acid, such as sodium acetate or sodium propionate.

The reaction temperature is generally determined by the melting point and the boiling point of the solvent in question. Preferably, the reaction is carried out at temperatures in the range from 0 to 100° C. and in particular in the range from 0 to 80° C.

In step ii), the 2-halo-3-(isothiazol-3-yl)aniline of the formula XI obtained in the reaction i) is reacted with an acylating agent R¹⁸—C(O)—L. Here, R¹⁸ has the meanings mentioned above. L is a customary leaving group.

Examples of acylating agents are carboxylic acids (L=OH), carboxylic esters, such as C₁-C₄-alkyl esters (L=C₁-C₄-alkyl, in particular methyl or ethyl), vinyl esters (L=CH═CH₂), 2-propenyl esters (L=C(CH₃)═CH₂), the acid anhydrides (L=O—C(O)—R¹⁸), acid halides, in particular acid chlorides (L=halogen, in particular chlorine), mixtures of the anhydrides R¹⁸—C(O)—O—C(O)—R¹⁸ with carboxylic acids, such as formic acid, and also mixed anhydrides (L=O—C(O)—R′ where R′=H or, for example, C₁-C₆-alkyl), for example a mixed anhydride with pivalic acid (R′=tert-butyl) or with formic acid (compounds of the formula H—C(O)—O—C(O)—R¹⁸).

The acylating agent is preferably employed in an amount of from 1.0 to 5 mol and in particular in an amount of from 1.0 to 2.0 mol, based on 1 mol of the compound XI.

If appropriate, an acidic or basic catalyst is employed in catalytic or stoichiometric amounts for the acylation of XI. The catalyst is preferably used in an amount of from 0.001 to 5 mol and in particular in an amount of from 0.01 to 1.2 mol, based on 1 mol of the compound XI.

Examples of basic catalysts are nitrogen bases, for example trialkylamines, such as triethylamine, pyridine compounds, such as pyridine itself or dimethylaminopyridine, furthermore oxo bases, such as sodium carbonate or potassium carbonate or the hydroxides of sodium, potassium or calcium.

Examples of acidic catalysts are, in particular, mineral acids, such as sulfuric acid.

The acylation is usually carried out in a solvent. Suitable solvents are, if appropriate, the liquid acylating agent itself or, if appropriate, the liquid catalyst. Suitable solvents are furthermore inert organic solvents, for example hydrocarbons, such as hexane or toluene, halogenated hydrocarbons, such as dichloromethane, trichloromethane, 1,2-dichloroethane or chlorobenzene, furthermore ethers, such as dioxane, tetrahydrofuran, methyl tert-butyl ether or dimethoxyethane.

In a preferred embodiment of this process step, the reaction of XI is carried out in a liquid anhydride in the presence of concentrated sulfuric acid. In another embodiment, the reaction is carried out in a two-phase system consisting of water and a water-immiscible organic solvent. This embodiment is suitable in particular in cases where solid acylating agents, for example solid acid chlorides, are used. In this case, the catalysts employed are frequently basic catalysts, in particular inorganic bases.

In a further preferred embodiment of this process step, the reaction of XI with an anhydride (R¹⁸—CO)₂O or R¹⁸—CO—O—CHO or a carboxylic acid R¹⁸—COOH is carried out in the presence of concentrated sulfuric acid in an inert solvent. In general, this variant requires smaller amounts of acylating agents, for example from 1 to 1.5 mol per mole of the compound XI. This variant gives, surprisingly, directly, in good yields and with high selectivity, the mono-N-acyl compounds X, without any significant amounts of the N,N-diacyl compounds XII being formed.

In the acylation of XI, the diacyl compound of the formula XII is frequently also formed, in addition to the anilide X. Depending on how the reaction is carried out, the diacyl compound of the formula XII may also be the only reaction product. In this case, the diacyl compound XII is, if appropriate in a mixture with the compound X, subjected to partial solvolysis. Here, the compound XII is cleaved into the compound X and a carboxylic acid R¹⁸—COOH, its salt or a derivative, for example an ester R¹⁸—COOR′ (R′ e.g.=C₁-C₄-alkyl).

Suitable agents for the solvolysis are, for example, water or alcohols, for example C₁-C₄-alkanols, such as methanol, ethanol or isopropanol, or mixtures of these alcohols with water.

The partial solvolysis of XII is preferably carried out in the presence of an acidic or basic catalyst. Examples of basic catalysts are the alkali metal hydroxides, such as sodium hydroxide or potassium hydroxide, or the alkoxides of C₁-C₄-alkanols, in particular sodium methoxide or potassium methoxide or sodium ethoxide or potassium ethoxide. Examples of acidic catalysts are mineral acids, such as hydrochloric acid or sulfuric acid.

The solvolysis catalyst is usually employed in an amount of from 0.1 to 5 mol per mole of the compound XII. In a preferred variant of this process step, the catalyst is employed in an amount of at least 0.5 mol/mole of compound XII and in particular in an approximately equimolar amount or in a molar excess, preferably of up to 2 mol, based on the compound XII.

Preferred agents for the solvolysis are C₁-C₄-alkanols. Preferred catalysts are the alkali metal hydroxides or the alkali metal C₁-C₄-alkoxides, such as sodium hydroxide, sodium methoxide and sodium ethoxide.

The partial solvolysis is usually carried out in a solvent. Suitable solvents are, in particular, the solvolysis agents themselves, for example the C₁-C₄-alkanols or mixtures of these solvolysis agents with inert solvents. Examples of inert solvents are the solvents mentioned above.

In a preferred embodiment of the present invention, the solvolysis of XII to give X is carried out in a C₁-C₄-alkanol in the presence of the corresponding alkoxide, preferably in methanol or ethanol with sodium methoxide or sodium ethoxide.

The solvolysis temperature is frequently above 0° C. and is generally limited only by the boiling point of the solvent. The reaction temperature is preferably in the range from 0 to 100° C. and in particular in the range from 20 to 80° C.

The products XI, XII and X obtained in steps i), ii) and iii) can be isolated using the work-up methods customary for this purpose. If appropriate, the reaction products of the reaction ii) can be used for the subsequent step iii) without further work-up. Frequently, the crude product of the compound X obtained in reaction ii) or iii) is, prior to the cyclization to the benzoxazole I-D, subjected to purification by crystallization and/or chromatography.

Work-up of the reaction mixtures is usually carried out in a manner known per se. Unless indicated otherwise in the processes described above, the products of value are obtained for example by dilution of the reaction solution with water and subsequent isolation of the product by filtration, crystallization or solvent extraction, or by removing the solvent, partitioning the residue in a mixture of water and a suitable organic solvent and work-up of the organic phase to afford the product.

The 3-arylisothiazoles of the formula I can be obtained in the preparation as isomer mixtures; however, if desired, these can be separated into substantially pure isomers using methods customary for this purpose, such as crystallization or chromatography, including chromatography on an optically active absorbate. Pure optically active isomers can be prepared advantageously from suitable optically active starting materials.

Agriculturally useful salts of the compounds I can be formed by reaction with a base of the corresponding cation, preferably an alkali metal hydroxide or hydride, or by reaction with an acid of the corresponding anion, preferably hydrochloric acid, hydrobromic acid, sulfuric acid, phosphoric acid or nitric acid.

Salts of I where the metal ion is not an alkali metal ion can also be prepared by cation exchange of the corresponding alkali metal salt in a conventional manner, similarly ammonium, phosphonium, sulfonium and sulfoxonium salts by means of ammonia, phosphonium, sulfonium or sulfoxonium hydroxides.

The compounds I and their agriculturally useful salts are suitable, both in the form of isomer mixtures and in the form of the pure isomers, for use as herbicides. The herbicidal compositions comprising the compounds I or salts thereof control vegetation on non-crop areas very efficiently, especially at high rates of application. They act against broad-leaved weeds and weed grasses in crops such as wheat, rice, maize, soya and cotton without causing any significant damage to the crop plants. This effect is mainly observed at low rates of application.

Depending on the application method used in each case, the compounds I, or compositions comprising them, can additionally be employed in a further number of crop plants for eliminating undesirable plants. Examples of suitable crops are the following: Allium cepa, Ananas comosus, Arachis hypogaea, Asparagus officinalis, Beta vulgaris spec. altissima, Beta vulgaris spec. rapa, Brassica napus var. napus, Brassica napus var. napobrassica, Brassica rapa var. silvestris, Camellia sinensis, Carthamus tinctorius, Carya illinoinensis, Citrus limon, Citrus sinensis, Coffea arabica (Coffea canephora, Coffea liberica), Cucumis sativus, Cynodon dactylon, Daucus carota, Elaeis guineensis, Fragaria vesca, Glycine max, Gossypium hirsutum, (Gossypium arboreum, Gossypium herbaceum, Gossypium vitifolium), Helianthus annuus, Hevea brasiliensis, Hordeum vulgare, Humulus lupulus, Ipomoea batatas, Juglans regia, Lens culinaris, Linum usitatissimum, Lycopersicon lycopersicum, Malus spec., Manihot esculenta, Medicago sativa, Musa spec., Nicotiana tabacum (N.rustica), Olea europaea, Oryza sativa, Phaseolus lunatus, Phaseolus vulgaris, Picea abies, Pinus spec., Pisum sativum, Prunus avium, Prunus persica, Pyrus communis, Ribes sylvestre, Ricinus communis, Saccharum officinarum, Secale cereale, Solanum tuberosum, Sorghum bicolor (s. vulgare), Theobroma cacao, Trifolium pratense, Triticum aestivum, Triticum durum, Vicia faba, Vitis vinifera, Zea mays.

In addition, the compounds I may also be used in crops which tolerate the action of herbicides owing to breeding, including genetic engineering methods.

Moreover, the 3-arylisothiazoles of the formula according to the invention and their agriculturally useful salts are also suitable for the desiccation and/or defoliation of plants.

As desiccants, they are suitable, in particular, for desiccating the above-ground parts of crop plants such as potatoes, oilseed rape, sunflowers and soya beans. This allows completely mechanical harvesting of these important crop plants.

Also of economic interest is

the coordinated dehiscence of fruits or the reduction of their adherence to the plant, for example in citrus fruit, olives or other kinds and species of pernicious fruit, stone fruit and nuts, since this facilitates harvesting of these fruits, and also the controlled defoliation of useful plants, in particular cotton. The dehiscence which is promoted by the application of active compounds of the formula I according to the invention and their agriculturally useful salts is due to the formation of abscission tissue between the fruit or leaf and shoot part of the plants. The defoliation of cotton is of very particular economic interest since it facilitates harvesting. Simultaneously, the shortening of the window within which the individual plants mature leads to increased quality of the harvested fiber material.

The compounds of the formula I according to the invention, or the herbicidal compositions comprising them, can be used, for example, in the form of ready-to-spray aqueous solutions, powders, suspensions, also highly-concentrated aqueous, oily or other suspensions or dispersions, emulsions, oil dispersions, pastes, dusts, materials for broadcasting, or granules, by means of spraying, atomizing, dusting, broadcasting, pouring or treating the seed or mixing with the seed. The use forms depend on the intended aims; in each case, they should ensure a very fine distribution of the active compounds according to the invention. The compositions according to the invention comprise a herbicidally effective amount of at least one compound of the formula I or an agriculturally useful salt of I and auxiliaries which are customary for formulating crop protection agents.

Suitable inert additives are essentially: mineral oil fractions of medium to high boiling point, such as kerosene and diesel oil, furthermore coal tar oils and oils of vegetable or animal origin, aliphatic, cyclic and aromatic hydrocarbons, e.g. paraffin, tetrahydronaphthalene, alkylated naphthalenes and their derivatives, alkylated benzenes and their derivatives, alcohols such as methanol, ethanol, propanol, butanol and cyclohexanol, ketones such as cyclohexanone, strongly polar solvents, e.g. amides such as N-methylpyrrolidone, and water.

Aqueous use forms can be prepared from emulsion concentrates, suspensions, pastes, wettable powders or water-dispersible granules by adding water. To prepare emulsions, pastes or oil dispersions, the compounds I, either as such or dissolved in an oil or solvent, can be homogenized in water by means of a wetting agent, tackifier, dispersant or emulsifier. Alternatively, it is possible to prepare concentrates comprising active compound, wetting agent, tackifier, dispersant or emulsifier and, if desired, solvent or oil, which are suitable for dilution with water.

Suitable surfactants are the alkali metal salts, alkaline earth metal salts and ammonium salts of aromatic sulfonic acids, e.g. ligno-, phenol-, naphthalene- and dibutylnaphthalenesulfonic acid, and of fatty acids, alkyl- and alkylarylsulfonates, alkyl sulfates, lauryl ether sulfates and fatty alcohol sulfates, and salts of sulfated hexa-, hepta- and octadecanols, and also of fatty alcohol glycol ethers, condensates of sulfonated naphthalene and its derivatives with formaldehyde, condensates of naphthalene, or of the naphthalenesulfonic acids with phenol and formaldehyde, polyoxyethylene octylphenol ether, ethoxylated isooctyl-, octyl- or nonylphenol, alkylphenyl or tributylphenyl polyglycol ether, alkylaryl polyether alcohols, isotridecyl alcohol, fatty alcohol/ethylene oxide condensates, ethoxylated castor oil, polyoxyethylene alkyl ethers or polyoxypropylene alkyl ethers, lauryl alcohol polyglycol ether acetate, sorbitol esters, lignosulfite waste liquors or methylcellulose.

Powders, materials for broadcasting and dusts can be prepared by mixing or grinding the active compounds together with a solid carrier.

Granules, e.g. coated granules, impregnated granules and homogeneous granules, can be prepared by binding the active compounds to solid carriers. Solid carriers are mineral earths, such as silicas, silica gels, silicates, talc, kaolin, limestone, lime, chalk, bole, loess, clay, dolomite, diatomaceous earth, calcium sulfate, magnesium sulfate, magnesium oxide, ground synthetic materials, fertilizers such as ammonium sulfate, ammonium phosphate and ammonium nitrate, ureas, and products of vegetable origin, such as cereal meal, tree bark meal, wood meal and nutshell meal, cellulose powders, or other solid carriers.

The concentrations of the active compounds I in the ready-to-use preparations can be varied within wide ranges. In general, the formulations comprise from 0.001 to 98% by weight, preferably 0.01 to 95% by weight, of at least one active compound. The active compounds are employed in a purity of from 90% to 100%, preferably 95% to 100% (according to the NMR spectrum).

The compounds I according to the invention can be formulated, for example, as follows:

I 20 parts by weight of the compound No. IAa.10 (cf. Table 1) are dissolved in a mixture composed of 80 parts by weight of alkylated benzene, 10 parts by weight of the adduct of 8 to 10 mol of ethylene oxide to 1 mol of oleic acid N-monoethanolamide, 5 parts by weight of calcium dodecylbenzenesulfonate and 5 parts by weight of the adduct of 40 mol of ethylene oxide to 1 mol of castor oil. Pouring the solution into 100,000 parts by weight of water and finely distributing it therein gives an aqueous dispersion which comprises 0.02% by weight of the active compound.

II 20 parts by weight of the compound No. IAa.14 are dissolved in a mixture composed of 40 parts by weight of cyclohexanone, 30 parts by weight of isobutanol, 20 parts by weight of the adduct of 7 mol of ethylene oxide to 1 mol of isooctylphenol and 10 parts by weight of the adduct of 40 mol of ethylene oxide to 1 mol of castor oil. Pouring the solution into 100,000 parts by weight of water and finely distributing it therein gives an aqueous dispersion which comprises 0.02% by weight of the active compound.

III 20 parts by weight of the active compound No. IAa.22 are dissolved in a mixture composed of 25 parts by weight of cyclohexanone, 65 parts by weight of a mineral oil fraction of boiling point 210 to 280° C. and 10 parts by weight of the adduct of 40 mol of ethylene oxide to 1 mol of castor oil. Pouring the solution into 100,000 parts by weight of water and finely distributing it therein gives an aqueous dispersion which comprises 0.02% by weight of the active compound.

IV 20 parts by weight of the active compound No. IAa.10 are mixed thoroughly with 3 parts by weight of sodium diisobutylnaphthalenesulfonate, 17 parts by weight of the sodium salt of a lignosulfonic acid from a sulfite waste liquor and 60 parts by weight of pulverulent silica gel, and the mixture is ground in a hammer mill. Finely distributing the mixture in 20,000 parts by weight of water gives a spray mixture which comprises 0.1% by weight of the active compound.

V 3 parts by weight of the active compound No. IAa.727 (R enantiomer) are mixed with 97 parts by weight of finely divided kaolin. This gives a dust which comprises 3% by weight of the active compound.

VI 20 parts by weight of the active compound No. IAa.22 are mixed intimately with 2 parts by weight of calcium dodecylbenzenesulfonate, 8 parts by weight of fatty alcohol polyglycol ether, 2 parts by weight of the sodium salt of a phenol/urea/formaldehyde condensate and 68 parts by weight of a paraffinic mineral oil. This gives a stable oily dispersion.

VII 1 part by weight of the compound No. IAa.727 (R enantiomer) is dissolved in a mixture composed of 70 parts by weight of cyclohexanone, 20 parts by weight of ethoxylated isooctylphenol and 10 parts by weight of ethoxylated castor oil. This gives a stable emulsion concentrate.

VIII 1 part by weight of the compound No. IAa.14 is dissolved in a mixture composed of 80 parts by weight of cyclohexanone and 20 parts by weight of Wettol® EM 31 (nonionic emulsifier based on ethoxylated castor oil). This gives a stable emulsion concentrate.

The herbicidal compositions or the active compounds comprising the 3-arylisothiazoles of the formula I and/or their salts can be applied pre- or post-emergence or together with the seed of a crop plant. It is also possible to apply the herbicidal compositions or the active compounds by sowing seeds of a crop plant which have been pre-treated with the herbicidal compositions or active compounds. If the active compounds are less well tolerated by certain crop plants, application techniques may be used in which the herbicidal compositions are sprayed, with the aid of the spraying equipment, in such a way that they come into as little contact as possible, if any, with the leaves of the sensitive crop plants, while the active compounds reach the leaves of undesirable plants growing underneath, or the bare soil surface (post-directed, lay-by).

The rates of application of active compound are from 0.001 to 3.0, preferably 0.01 to 1.0, kg/ha of active substance (a.s.), depending on the control target, the season, the target plants and the growth stage.

To widen the spectrum of action and to achieve synergistic effects, the compounds of the formula I according to the invention may be mixed with a large number of representatives of other herbicidal or growth-regulating active compound groups and applied concomitantly. Suitable components for mixtures are, for example, 1,2,4-thiadiazoles, 1,3,4-thiadiazoles, amides, aminophosphoric acid and its derivatives, aminotriazoles, anilides, aryloxy/hetaryloxyalkanoic acids and their derivatives, benzoic acid and its derivatives, benzothiadiazinones, 2-aroyl-1,3-cyclohexanediones, hetaryl aryl ketones, benzylisoxazolidinones, meta-CF₃-phenyl derivatives, carbamates, quinolinecarboxylic acid and its derivatives, chloroacetanilides, cyclohexane-1,3-dione derivatives, diazines, dichloropropionic acid and its derivatives, dihydrobenzofurans, dihydrofuran-3-ones, dinitroanilines, dinitrophenols, diphenyl ethers, dipyridyls, halocarboxylic acids and their derivatives, ureas, 3-phenyluracils, imidazoles, imidazolinones, N-phenyl-3,4,5,6-tetrahydrophthalimides, oxadiazoles, oxiranes, phenols, aryloxy- and hetaryloxyphenoxypropionic esters, phenylacetic acid and its derivatives, phenylpropionic acid and its derivatives, pyrazoles, phenylpyrazoles, pyridazines, pyridinecarboxylic acid and its derivatives, pyrimidyl ethers, sulfonamides, sulfonylureas, triazines, triazinones, triazolinones, triazolecarboxamides and uracils.

It may furthermore be advantageous to apply the compounds I, alone or else concomitantly in combination with other herbicides, in the form of a mixture with other crop protection agents, for example together with agents for controlling pests or phytopathogenic fungi or bacteria. Also of interest is the miscibility with mineral salt solutions, which are employed for treating nutritional and trace element deficiencies. Non-phytotoxic oils and oil concentrates may also be added.

The examples below serve to illustrate the invention:

### I PREPARATION EXAMPLES

The exemplary compounds I (Examples 1 to 6) were prepared from methyl 4-chloroisothiazole-5-carboxylates which for their part were prepared similarly to the processes described in the literature. In this context, see also the synthesis sequence described in Example 1 (steps 1.1 to 1.7), and the methods described in

U.S. Pat. No. 4,544,752, U.S. Pat. No. 4,346,094 (steps 1.4 to 1.7)

J. Org. Chem. 28 (1963), 2436 (step 1.4)

Houben-Weyl 10/4, p. 31 (step 1.4)

Liebigs Ann. Chem. 1979, 1534-1546 (step 1.5)

J. Heterocyclic Chem. 24 (1987), 243-245 (step 1.6)

and the literature cited therein, which methods are included in their entirety into the present invention by way of reference.

Hereinbelow, the abbreviation Me denotes methyl.

### 3-(4-Chloro-2-fluoro-5-methoxyphenyl)-4-chloro-5-trifluoro-methylisothiazole (Example 1)

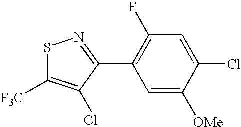

### 1.1 4-Chloro-2-fluoro-5-methoxybenzyl alcohol (1)

Over a period of 2 h, 300 ml (300 mmol) of a solution of BH₃.SMe₂ (1 M solution in dichloromethane) were added dropwise to a solution of 46.5 g (227 mmol) of 4-chloro-2-fluoro-5-methoxybenzoic acid in 500 ml of tetrahydrofuran, and the reaction mixture was stirred at room temperature for 3 days. Excess BH₃ was hydrolyzed by slow dropwise addition of 200 ml of water, with ice-cooling, and the pH was then adjusted to pH 2 using hydrochloric acid and the mixture was extracted twice with 200 ml of ethyl acetate. The organic phases were dried over magnesium sulfate and concentrated under reduced pressure, and twice, toluene was added and the solvent was removed again under reduced pressure. This gave 40.6 g (94%) of the benzyl alcohol 1.

¹H-NMR (CDCl₃, 270 MHz): δ(ppm)=3.9 (s, 3H, OMe), 4.7 (s, 2H, CH₂OH), 7.0 (d, 1H, Ar—H), 7.1 (d, 1H, Ar—H).

### 1.2 4-Chloro-2-fluoro-5-methoxybenzyl bromide (2)

At 0-5° C., 58.7 g (224 mmol) of triphenylphosphine were added to a solution of 38.8 g (204 mmol) of 4-chloro-2-fluoro-5-methoxybenzyl alcohol 1 in 600 ml of tetrahydrofuran, and after 10 minutes, a solution of 74.4 g (224 mmol) of carbon tetrabromide in 300 ml of tetrahydrofuran was added slowly (over a period of 30 minutes). The reaction mixture was stirred at room temperature over the weekend, concentrated under reduced pressure and then filtered through a short silica gel column (mobile phase cyclohexane/ethyl acetate=2:1). Following concentration under reduced pressure, the crude product was distilled under reduced pressure (b.p. 89° C. at 0.26 mbar). This gave 33.9 g (66%) of the benzyl bromide 2.

¹H-NMR (CDCl₃, 270 MHz): δ(ppm)=3.9 (s, 3H, OMe), 4.5 (s, 2H, CH₂Br), 6.9 (d, 1H, Ar—H), 7.15 (d, 1H, Ar—H).

### 1.3 4-Chloro-2-fluoro-5-methoxybenzyl cyanide (3)

6.6 g (134 mmol) of dried sodium cyanide (6 h at 110° C. under reduced pressure) and a spatula tip of sodium iodide were added to a solution of 22.6 g (89.2 mmol) of 4-chloro-2-fluoro-5-methoxybenzyl bromide 2 in 600 ml of triethylene glycol which had been dried over molecular sieves. The reaction mixture was stirred at 100° C. for 40 minutes and, after cooling, introduced into 3 l of water. The aqueous phase was extracted twice with dichloromethane. The dichloromethane phase was dried over magnesium sulfate and concentrated, giving 19 g of the benzyl cyanide 3. The aqueous phase was then extracted three more times with ethyl acetate. The organic phases were washed once with water, dried over magnesium sulfate and concentrated under reduced pressure. This gave an additional 7.8 g of product. The product still contains relatively large amounts of triethylene glycol; however, these do not interfere with the subsequent reaction.

1H-NMR (CDCl₃, 270 MHz): δ(ppm)=3.7 (s, 2H, CH₂CN), 3.9 (s, 3H, OMe), 6.95 (d, 1H, Ar—H), 7.1 (d, 1H, Ar—H).

### 1.4 (4-Chloro-2-fluoro-5-methoxyphenyl)-N-tosyloximinoacetonitrile (5)

5 ml of anhydrous ethanol were added to 0.50 g (11.6 mmol) of sodium hydride (60 percent). After 15 minutes, a solution of 2.1 g (10.5 mmol) of the benzyl nitrile 3 in 25 ml of ethanol was added dropwise at 0-5° C. over a period of 30 minutes, and the mixture was stirred for another 20 minutes at the same temperature. 1.4 g (11.6 mmol) of n-pentyl nitrite were then added dropwise at 0-5° C. over a period of 10 minutes, and the mixture was allowed to react at room temperature overnight. The mixture was concentrated under reduced pressure, 100 ml of diethyl ether were added and the resulting precipitate was then filtered off with suction and dried. This gave 1.9 g (72.2%) of the sodium salt 4 of the oxime which was immediately, without purification, converted into the oxime tosylate.

A solution of 1.9 g (7.6 mmol) of the resulting oxime sodium salt 4 in 40 ml of DMF was admixed with 1.4 g (7.6 mmol) of tosyl chloride. The reaction mixture was heated at 70-75° C. for 30 minutes and, after cooling, stirred into 1 l of water. The mixture was extracted three times with methyl tert-butyl ether and the organic phases were washed once with 250 ml of water and then dried over magnesium sulfate. Concentration gave 1.52 g (52%) of the oxime tosylate 5 as Z/E mixture (5a and 5b) in a ratio of 60:40.

¹H-NMR (CDCl₃, 400 MHz): 5a, 5b: δ(ppm)=2.5 (2d, 3H each, Me, 5a+5b), 3.9 (2s, 3H each, OMe, 5a+5b), 6.9 (d, 1H, Ar—H, 5b), 7.1 (d, 1H, Ar—H, 5a), 7.25 (2d, 1H each, Ar—H, 5a+5b), 7.4 (2d, 2H each, Ar—H, 5a+5b), 7.9 (d, 2H, Ar—H, 5b), 7.95 (d, 2H, Ar—H, 5a).

### 1.5 Methyl 3-(4-chloro-2-fluoro-5-methoxyphenyl)-4-aminoisothiazole-5-carboxylate (6)

550 mg (5.2 mmol) of methyl thioglycolate were added to a suspension of 1.52 g (4 mmol) of oxime tosylate 5 in 20 ml of ethanol, and a solution of 520 mg (6 mmol) of morpholine in ethanol was then added dropwise over a period of 10 minutes. The mixture was stirred at room temperature for 2 days, 150 ml of water were added, the mixture was stirred for another 30 minutes and the resulting precipitate was filtered off with suction. Drying gave 620 mg (49%) of the methyl isothiazole-5-carboxylate 6 of melting point 121-124° C.

¹H-NMR (CDCl₃, 400 MHz): δ(ppm)=3.9 (2s, 3H each, OMe and COOMe), 5.4 (bs, NH₂), 7.1 (d, 1H, Ar—H), 7.3 (d, 1H, Ar—H).

### 1.6 Methyl 3-(4-chloro-2-fluoro-5-methoxyphenyl)-4-chloroisothiazole-5-carboxylate (7)

At room temperature, a suspension of 4.0 g (12.6 mmol) of aminoisothiazole 6 in 100 ml of acetonitrile was added over a period of 30 minutes to a solution of 2.1 g (15.8 mmol) of CuCl₂ and 2.0 g (19.0 mmol) of tert-butyl nitrite in 50 ml of acetonitrile, and the mixture was stirred at room temperature overnight. The mixture was concentrated under reduced pressure and the crude product was then purified by column chromatography (silica gel—cyclohexane/ethyl acetate). This gave 2.1 g (50%) of the chloro compound 7 (m.p. 131-132° C.). Furthermore, 1.1 g (29%) of methyl 3-(4-chloro-2-fluoro-5-methoxyphenyl)isothiazole-5-carboxylate 8 (m.p. 139-142° C.) were obtained.

¹H-NMR (CDCl₃, 270 MHz): 7: δ(ppm)=3.9 (s, 3H, OMe or COOMe), 4.0 (s, 3H, OMe or COOMe), 7.0 (d, 1H, Ar—H), 7.3 (d, 1H, Ar—H).

¹H-NMR (CDCl₃, 270 MHz): 8: δ(ppm)=4.0 (2s, 3H each, OMe and COOMe), 7.25 (d, 1H, Ar—H), 7.75 (d, 1H, Ar—H), 8.25 (d, 1H, isothiazole-H).

### 1.7 3-(4-Chloro-2-fluoro-5-methoxyphenyl)-4-chloroisothiazole-5-carboxylic acid (9)

A suspension of 2.6 g (7.7 mmol) of methyl 3-(4-chloro-2-fluoro-5-methoxyphenyl)-4-chloroisothiazole-5-carboxylate 7 in 100 ml of methanol was admixed with a solution of 0.34 g (8.5 mmol) of NaOH in 20 ml of water, and the mixture was stirred at room temperature overnight. The methanol was removed under reduced pressure and the alkaline aqueous phase was then extracted with 250 ml of ethyl acetate and then adjusted to pH 1 using hydrochloric acid. The resulting precipitate was filtered off with suction and dried. This gave 1.3 g of the carboxylic acid 9. The filtrate was extracted three times with ethyl acetate and the extract was dried over magnesium sulfate, giving, after concentration, a further 0.2 g of the carboxylic acid 9 [overall yield 1.5 g (61%)].

¹H-NMR (DMSO, 270 MHz): δ(ppm)=3.9 (s, 3H, OMe), 7.3 (d, 1H, Ar—H), 7.7 (d, 1H, Ar—H).

### 1.8 3-(4-Chloro-2-fluoro-5-methoxyphenyl)-4-chloro-5-trifluoromethylisothiazole (compound IAa.7)

1.5 g (47 mmol) of the isothiazolecarboxylic acid from 1.7 were initially charged in an HC pressure container. 20 g of hydrogen fluoride (anhydrous) were then condensed into the container, 4 g of gaseous sulfur tetrafluoride were introduced under pressure and the mixture was stirred at 60° C. under autogenous pressure (3 to 4 bar) for 24 h. The container was vented and the reactor content was then poured onto 300 g of ice water, made alkaline with 50% strength aqueous sodium hydroxide solution and admixed with 150 ml of methylene chloride. The methylene chloride phase was separated off, washed with water, dried with magnesium sulfate and concentrated under reduced pressure. The residue was chromatographed over silica gel using a cyclohexane/ethyl acetate gradient. This gave 1.5 g of the title compound with a purity of 97.6% (GC) (90% of theory).

¹H-NMR (CDCl₃, 270 MHz): δ(ppm)=3.95 (s, 3H, OMe), 7.05 (d, 1H, Ar—H); 7.30 (d, 1H, Ar—H).

### 3-(4-Chloro-2-fluoro-5-hydroxyphenyl)-4-chloro-5-trifluoromethylisothiazole (Example 2; compound IAa.6)

At 0-5° C., 3.3 ml (3.3 mmol) of a boron tribromide solution (1 M in CH₂Cl₂) were added dropwise to a solution of 1.1 g (3.2 mmol) of compound IAa.7 from Example 1 in 40 ml of CH₂Cl₂, and the mixture was stirred at room temperature overnight. Another 3.3 ml (3.3 mmol) of the boron tribromide solution (1 M in CH₂Cl₂) were then added, and the mixture was stirred at room temperature for 4 h. 100 ml of ice-cold water were added to the reaction mixture, the phases were separated and the aqueous phase was extracted twice with 100 ml of dichloromethane. The combined organic phases were dried over magnesium sulfate and concentrated under reduced pressure. This gave 1.0 g (94%) of the hydroxy compound IAa.6.

¹H-NMR (CDCl₃, 270 MHz): δ(ppm)=5.6 (bs, OH), 7.15 (d, 1H, Ar—H), 7.25 (d, 1H, Ar—H).

### Methyl 2-[2-chloro-4-fluoro-5-(4-chloro-5-trifluoromethylisothiazol-3-yl)phenoxy]propionate as racemate (Example 3 compound IAa.22)

A solution of 308 mg (0.93 mmol) of the compound IAa.6 in 20 ml of DMF was admixed with 141 mg (1.02 mmol) of K₂CO₃ and then, at 0-5° C. and over a period of 2 h, with 170 mg (1.02 mmol) of racemic methyl 2-bromopropionate, and the mixture was stirred at room temperature overnight. The mixture was then concentrated to dryness under reduced pressure, 100 ml of water were added to the residue and the mixture was extracted twice with 100 ml of methyl tert-butyl ether. The combined organic phases were washed once with water, dried over magnesium sulfate and concentrated under reduced pressure. This gave 350 mg (90%) of the racemic methyl phenoxypropionate IAa.22.

¹H-NMR (CDCl₃, 270 MHz): δ(ppm)=1.7 [d, 3H, OCH(Me)COOMe], 3.8 (s, 3H, COOMe), 4.8 [q, 1H, OCH(Me)COOMe], 7.05 (d, 1H, Ar—H), 7.3 (d, 1H, Ar—H).

### Methyl 2-[2-chloro-4-fluoro-5-(4-chloro-5-trifluoromethylisothiazol-3-yl)phenoxy]propionate as R enantiomer (Example 4; compound IAa.727)

In the manner described in Example 3, compound IAa.6 was reacted with 2 equivalents of methyl (2S)-2-chloropropionate, giving the R enantiomer of IAa.22 in a yield of 81%.

¹H-NMR (CDCl₃, 270 MHz): δ(ppm)=1.7 [d, 3H, OCH(Me)COOMe], 3.8 (s, 3H, COOMe), 4.8 [q, 1H, OCH(Me)COOMe], 7.05 (d, 1H, Ar—H), 7.3 (d, 1H, Ar—H).

### 3-(4-Chloro-2-fluoro-5-propargyloxyphenyl)-4-chloro-5-trifluoromethylisothiazole (Example 5; compound IAa.10)

In the manner described in Example 3, compound IAa.6 was reacted with 1 equivalent of propargyl bromide, giving the title compound IAa.10 in a yield of 53%.

¹H-NMR (CDCl₃, 270 MHz): δ(ppm)=2.55 (t, 1H, C≡CH), 4.8 (d, 2H, OCH₂—C≡C), 7.2 (d, 1H, Ar—H), 7.3 (d, 1H, Ar—H).

### Methyl{2-chloro-5-[4-chloro-5-trifluoromethylisothiazol-3-yl]-4-fluorophenoxy}acetate (Example 6; compound IAa.14)

In the manner described in Example 3, compound IAa.6 was reacted with 1 equivalent of methyl bromoacetate, giving the title compound IAa.14 in a yield of 87%.

¹H-NMR (CDCl₃, 270 MHz): δ(ppm)=3.8 (s, 3H, COOMe), 4.7 (s, 2H, OCH₂COOMe), 7.0 (d, 1H, Ar—H), 7.35 (d, 1H, Ar—H).

TABLE 4
Compounds of the formula IAa where R³ = F and R⁴ = Cl;
Examples 1 to 6.

| | |
|---|---|
| | (IAa) |
| 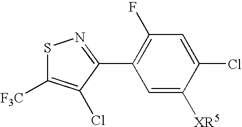 | |

| Example | No. | X—R⁵ | ¹H-NMR δ (ppm) |
|---|---|---|---|
| 1 | IAa.7 | O—CH₃ | 3.95, 7.05, 7.30 |
| 2 | IAa.6 | OH | 5.6, 7.15, 7.25 |
| 3 | IAa.22 | OCH(CH₃)COOCH₃ racem. | 1.7, 3.8, 4.8, 7.05, |
| | | | 7.3 |
| 4 | IAa.727 | OCH(CH₃)COOCH₃ R | 1.7, 3.8, 4.8, 7.05, |
| | | config. | 7.3 |
| 5 | IAa.10 | CH₂—C≡CH | 2.55, 4.8, 7.2, 7.3 |
| 6 | IAa.14 | OCH₂COOCH₃ | 3.8, 4.7, 7.0, 7.35 |

### 3-(4-Chlorophenyl)-5-trifluoromethylisothiazole (Example 7)

8.9 g (0.037 mol) of 3-(4-chlorophenyl)isothiazole-5-carboxylic acid, prepared by thermolytic reaction of 5-(4-chlorophenyl)-1,3,4-oxathiazol-2-one with methyl propiolate according to R. K. Howe et al. (loc. cit.), were initially charged in a 0.5 l HC autoclave. 45 g (2.25 mol) of anhydrous hydrogen fluoride were then condensed into the autoclave and 28 g of sulfur tetrafluoride were added under pressure. The mixture was stirred at 60° C. for 24 h. The autoclave was vented and the reactor content was then poured onto 500 g of ice, made alkaline with 50% strength aqueous sodium hydroxide solution and admixed with 350 ml of methylene chloride. The mixture was filtered through kieselguhr and the methylene chloride phase was then separated off and dried with magnesium sulfate. The methylene chloride phase was concentrated under reduced pressure and admixed with cyclohexane, whereupon the title compound precipitated as a solid. The solid was filtered off and the cyclohexane phase was concentrated further, resulting in the precipitation of more product. A total of 8 g (65%) of the title compound with a purity of 98.8% (GC) were obtained.

¹H-NMR (DMSO, 270 MHz): δ(ppm)=7.55 (d, 2H, aryl-H); 8.10 (d, 2H, aryl-H); 8.7 (s, 1H, isothiazole-H).

### 4-Chloro-3-(2,4-dichlorophenyl)-5-trifluoromethylisothiazole (Example 8; compound IAa.243)

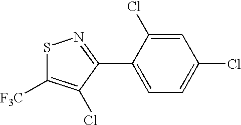

### 8.1 (2,4-Dichlorophenyl)tosyloximinoacetonitrile (11)

With ice cooling, a solution of 9.7 g (52.2 mmol) of 2,4-dichlorobenzylnitrile in 20 ml of dimethylformamide was added dropwise to a suspension of 2.3 g (57.4 mmol) of sodium hydroxide (60%) in 250 ml of dimethylformamide, with the reaction temperature being at most 20° C., and the mixture was stirred at 0-5° C. for another 20 min. 6.7 g (57.4 mmol) of n-pentyl nitrite were then added dropwise at 0-5° C. over a period of 30 minutes, and the mixture was stirred at this temperature for another 30 min. Cooling was removed, and a suspension of 21.9 g (114.7 mmol, 2 equivalents) of tosyl chloride in 30 ml of dimethylformamide was then added at room temperature to the reaction mixture, which was then heated to 70° C. and stirred at 70° C. for 3 hours. After cooling, the mixture was concentrated under reduced pressure and the oily residue was stirred into 1.5 l of water. 400 ml of methyl tert-butyl ether were added, and the mixture was stirred at room temperature for 20 minutes. The resulting precipitate was filtered off with suction and dried. This gave 10.8 g of the oxime tosylate 11. The phases of the filtrate were separated and the aqueous phase was extracted two more times with methyl tert-butyl ether. The combined organic phases were dried over magnesium sulfate and concentrated under reduced pressure. This gave an additional 10.4 g of product. Total yield: 21.2 g (>100%) of oxime tosylate 11 which contained small amounts of dimethylformamide.

¹H-NMR (CDCl₃, 270 MHz): δ(ppm)=2.5 (s, 3H, Me), 7.35 to 7.45 (m, 4H, Ar—H), 7.5 (d, 1H, Ar—H), 7.95 (d, 2H, Ar—H).

### 8.2 Methyl 4-amino-3-(2,4-dichlorophenyl)isothiazole-5-carboxylate (12)

7.2 g (67.9 mmol) of methyl thioglycolate were added to a suspension of 21.2 g (52.2 mmol) of oxime tosylate 11 from 8.1 in 300 ml of ethanol, and, over a period of 2 hours, 9.1 g (105 mmol) of morpholine were then added dropwise, with the temperature not exceeding 30° C. The mixture was stirred overnight at room temperature. Following concentration under reduced pressure, the crude product was purified chromatographically (silica gel—cyclohexane/ethyl acetate=6:1 to 1:1). This gave 7.5 g (47%, based on the 2,4-dichloro-benzylnitrile) of methyl 4-amino-3-(2,4-dichlorophenyl)-isothiazole-5-carboxylate 12.

¹H-NMR (CDCl₃, 270 MHz): δ(ppm)=3.9 (s, 3H, COOMe), 5.2 (bs, 2H, NH₂), 7.4 (s, 2H, Ar—H), 7.55 (s, 1H, Ar—H).

### 8.3 Methyl 4-chloro-3-(2,4-dichlorophenyl)isothiazole-5-carboxylate (13)

At 0-5° C., a solution of 2.1 g (30.9 mmol) of NaNO₂ in 20 ml of water was added dropwise to a solution of 8.5 g (28.1 mmol) of aminoisothiazole 12 in 100 ml of concentrated hydrochloric acid, and the mixture was stirred for another 10 min. Over a period of 15 min, this solution was added dropwise at 0-5° C. to a solution of 3.1 g (30.9 mmol) of copper(I) chloride in 100 ml of hydrochloric acid, and the mixture was stirred at the same temperature for another 10 min. The reaction mixture was then heated slowly (evolution of N₂) and heated at reflux for 2 hours. After cooling, the reaction mixture was stirred into 1 l of ice water and extracted three times with ethyl acetate. The combined organic phases were washed once with saturated NaCl solution, dried over magnesium sulfate and concentrated under reduced pressure. This gave 8.4 g (93%) of methyl 4-chloro-3-(2,4-dichlorophenyl)isothiazole-5-carboxylate 13 (purity according to ¹H-NMR: about 80-90%) which was used for the following reaction without purification. Moreover, methyl 3-(2,4-dichlorophenyl)-isothiazole-5-carboxylate (14) was obtained as a byproduct.

¹H-NMR (CDCl₃, 400 MHz): 13: δ(ppm)=4.0 (s, 3H, COOMe), 7.35 (m, 2H, Ar—H), 7.55 (s, 1H, Ar—H).

¹H-NMR (CDCl₃, 400 MHz): 14: δ(ppm)=4.0 (s, 3H, COOMe), 7.35 (m, 1H, Ar—H), 7.5 (d, 1H, Ar—H), 7.75 (d, 1H, Ar—H), 8.2 (s, 1H, isothiazole-H).

### 8.4 4-Chloro-3-(2,4-dichlorophenyl)isothiazole-5-carboxylic acid (15)

A solution of 1.1 g (28.3 mmol) of NaOH in 20 ml of water was added to a suspension of 8.3 g (25.7 mmol) of methyl 4-chloro-3-(2,4-dichlorophenyl)isothiazole-5-carboxylate 13 in 100 ml of methanol, and the mixture was stirred at room temperature for 16 h. The methanol was removed under reduced pressure, and 200 ml of water were then added and the alkaline aqueous phase was extracted with 200 ml of ethyl acetate. The aqueous phase was then adjusted to pH 1-2 using hydrochloric acid. The aqueous phase was extracted three times with ethyl acetate and the combined organic phases were washed once with water, dried over magnesium sulfate and concentrated under reduced pressure. This gave 6.9 g (87%) of 4-chloro-3-(2,4-dichlorophenyl)isothiazole-5-carboxylate 15 as a solid of melting point 193° C. (decomposition).

¹H-NMR (DMSO, 270 MHz): δ(ppm)=7.6 (m, 2H, Ar—H), 7.9 (d, 1H, Ar—H).

### 8.5 4-Chloro-3-(2,4-dichlorophenyl)-5-trifluoromethylisothiazole

12.2 g (40 mmol) of the isothiazolecarboxylic acid 15 from 8.4 were initially charged in a HC pressure container. 60 g (3.0 mol) of hydrogen fluoride (anhydrous) were then condensed in, 30.3 g (0.28 mol) of sulfur tetrafluoride were added under pressure and the mixture was stirred under intrinsic pressure (3 to 4 bar) at 60° C. for 24 hours. The reactor was vented and the reactor contents were poured onto 300 g of ice water and made alkaline using 50% strength aqueous sodium hydroxide solution, and 150 ml of methylene chloride were added. The methylene chloride phase was separated off, washed with water, dried with magnesium sulfate and concentrated under reduced pressure. The residue was chromatographed on silica gel using a cyclohexane/ethyl acetate gradient. This gave 4-chloro-3-(2,4-dichlorophenyl)-5-trifluoromethylisothiazole (compound IAa.243) in a yield of 77%.

¹H-NMR (CDCl₃, 270 MHz): δ(ppm)=7.35 (d, 1H , Ar—H); 7.4 (dd, 1H, Ar—H), 7.55 (d, 1H, Ar—H).

### 4-Chloro-3-(2,4-dichloro-5-nitrophenyl)-5-trifluoromethyl-isothiazole (Example 9; Compound IAa.246)

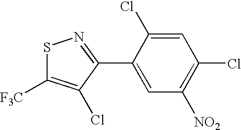

With ice cooling, 15 ml of fuming nitric acid were added dropwise to 15 ml of concentrated sulfuric acid. Over a period of 1 hour, 9.6 g (28.9 mmol) of the compound IAa.243 from Example 8 were then added a little at a time with ice cooling, the reaction temperature not exceeding 30° C., and the mixture was stirred at room temperature for 2 hours. The reaction mixture was then stirred into 300 ml of ice water and stirred for a further 2 hours. The resulting precipitate was filtered off with suction, dried and dissolved in 200 ml of ethyl acetate. The organic phase was washed twice with water, dried over magnesium sulfate and concentrated under reduced pressure. This gave 9.9 g (91%) of 4-chloro-3-(2,4-dichloro-5-nitrophenyl)-5-trifluoromethylisothiazole IAa.246 as a solid of melting point 104-106° C. which was used for the following reaction without further purification (purity:>90%). Crystallization from cyclohexane/ethyl acetate gave a pure sample of the nitro compound IAa.246.

¹H-NMR (CDCl₃, 270 MHz): δ(ppm)=7.8 (s, 1H, Ar—H); 8.05 (s, 1H, Ar—H).

### 2,4-Dichloro-5-(4-chloro-5-trifluoromethyl-3-isothiazolyl)aniline (Example 10; IAa.247)

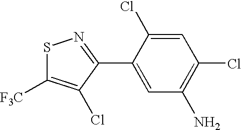

A suspension of 5.0 g (89.3 mmol) of iron powder in 10 ml of water and 1 ml of glacial acetic acid was heated at reflux. 50 ml of n-propanol were added dropwise to this suspension, followed by 9.4 g (25 mmol) of the compound IAa.246 from Example 9, a little at a time over a period of 10 min. The mixture was then stirred under reflux for 3 hours. After cooling, the reaction mixture was concentrated under reduced pressure. 200 ml of ethyl acetate and a spatula tip of activated carbon were added to the residue. After filtration through Celite, the filtrate was concentrated. This gave 8.6 g (99%) of the amino compound IAa.247.

¹H-NMR (CDCl₃, 400 MHz): δ(ppm)=4.2 (bs, 2H, NH₂), 6.8 (s, 1H, Ar—H), 7.4 (s, 1H, Ar—H).

### N-{2,4-Dichloro-5-[4-chloro-5-(trifluoromethyl)-3-isothiazolyl]-phenyl}-N-(ethylsulfonyl)ethanesulfonamide (Example 11; compound IAa.769)

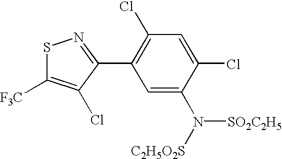

1.08 g (10.8 mmol) of triethylamine, 100 mg of dimethylaminopyridine and 1.08 g (8.4 mmol) of ethanesulfonyl chloride were added to a solution of 890 mg (2.6 mmol) of the compound IAa.247 from Example 10 in 40 ml of CH₂Cl₂, and the mixture was stirred at room temperature for three days. The mixture was concentrated under reduced pressure and the residue was then chromatographed (cyclohexane:ethyl acetate=9:1). This gave 970 mg (70%) of the title compound of melting point 153-154° C.

¹H-NMR (CDCl₃, 270 MHz): δ(ppm)=1.5 (t, 6H, CH₃), 3.55-3.85 (m, 4H, CH₂), 7.5 (s, 1H, Ar—H), 7.75 (s, 1H, Ar—H).

### N-{2,4-Dichloro-5-[4-chloro-5-(trifluoromethyl)-3-isothiazolyl]-phenyl}ethanesulfonamide (Example 12; compound IAa.770)

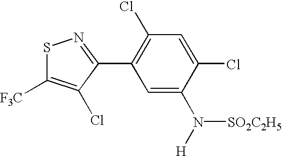

320 mg (1.8 mmol) of a 39% strength solution of sodium methoxide in methanol were added dropwise to a solution of 940 mg (1.8 mmol) of the compound IAa.769 from Example 11 in 30 ml of methanol. The reaction mixture was stirred at room temperature for 4 hours, adjusted to pH 6 using 10% strength hydrochloric acid and concentrated under reduced pressure.

Column chromatography gave 600 mg (76%) of the title compound of melting point 135° C.

¹H-NMR (CDCl₃, 270 MHz): δ(ppm)=1.4 (t, 3H, CH₃), 3.2 (q, 2H, CH₂), 6.8 (s, 1H, NH), 7.6 (s, 1H, Ar—H), 7.75 (s, 1H, Ar—H).

### N-{2,4-Dichloro-5-[4-chloro-5-(trifluoromethyl)-3-isothiazolyl]-phenyl}methanesulfonamide (Example 13; compound IAa.361)

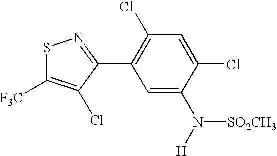

Analogously to Examples 11 and 12, the methanesulfonamide IAa.361 of melting point 161-164° C. was prepared from the compound IAa.247 from Example 10.

¹H-NMR (CDCl₃, 270 MHz): δ(ppm)=3.1 (s, 3H, CH₃), 6.9 (bs, 1H, NH), 7.65 (s, 1H, Ar—H), 7.75 (s, 1H, Ar—H).

TABLE 5
Compounds of the formula IAa where R³ = Cl and R⁴ = Cl;
Examples 8 to 13.

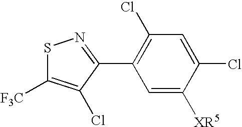

| Example | No. | X—R⁵ | ¹H-NMR δ (ppm) |
|---|---|---|---|
| 8 | IAa.243 | H | 7.35, 7.4, 7.55 |
| 9 | IAa.246 | NO₂ | 7.8, 8.05 |
| 10 | IAa.247 | NH₂ | 4.2, 6.8, 7.4 |
| 11 | IAa.769 | N(SO₂C₂H₅)₂ | 1.5, 3.55-3.85, |
| | | | 7.5, 7.75 |
| 12 | IAa.770 | NHSO₂C₂H₅ | 1.4, 3.2, 6.8, |
| | | | 7.6, 7.75 |
| 13 | IAa.361 | NHSO₂Me | 3.1, 6.9, 7.65, |
| | | | 7.75 |

### 4,6-Dichloro-7-(4-chloro-5-trifluoromethyl-3-isothiazolyl)-2-ethyl-1,3-benzoxazole (Example 14; compound IDa.55)

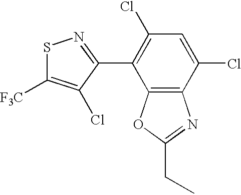

### 14.1 2-Bromo-4,6-dichloro-3-(4-chloro-5-trifluoromethyl-3-isothiazolyl)aniline (16)

4.1 g (50 mmol) of sodium acetate and then, at room temperature, 1.6 g (10 mmol) of bromine were added to a solution of 3.5 g (10 mmol) of the compound IAa.247 from Example 10 in 50 ml of acetic acid, and the mixture was stirred at room temperature overnight. 100 ml of saturated sodium bicarbonate solution and 150 ml of ethyl acetate (evolution of gas) were added dropwise to the reaction mixture, which was then stirred for another 10 min. The phases were separated and the aqueous phase was then extracted twice with ethyl acetate. The combined organic phases were washed with saturated sodium bicarbonate solution until neutral, dried over magnesium sulfate, filtered through silica gel and concentrated under reduced pressure. This gave 4.1 g (96%) of 2-bromo-4,6-dichloro-3-(4-chloro-5-trifluoro-methyl-3-isothiazolyl)aniline 16 as a solid of melting point 77-78° C.

¹H-NMR (CDCl₃, 270 MHz): δ(ppm)=4.65 (s, 2H, NH₂), 7.4 (s, 1H, Ar—H).

### 14.2 N-[2-Bromo-4,6-dichloro-3-(4-chloro-5-trifluoromethyl-3-isothiazolyl)phenyl]propanamide (17)

0.5 g (3.9 mmol) of propionic anhydride and a drop of sulfuric acid were added to a solution of 1.5 g (3.5 mmol) of 2-bromo-4,6-dichloro-3-(4-chloro-5-trifluoromethyl-3-isothiazolyl)aniline 16 in 50 ml of toluene, and the mixture was stirred at room temperature for 48 hours. The resulting precipitate was filtered off and washed with methyl tert-butyl ether. The filtrate was concentrated under reduced pressure and the crude product was dissolved in 50 ml of ethyl acetate, 40 ml of water were added, the pH was adjusted to 10 using 2N NaOH and the mixture was stirred at room temperature for 10 min. The phases were separated and the aqueous phase was extracted twice with ethyl acetate. The combined organic phases were dried over magnesium sulfate and then concentrated under reduced pressure. This gave 1.35 g (80%) of N-[2-bromo-4,6-dichloro-3-(4-chloro-5-trifluoro-methyl-3-isothiazolyl)phenyl]propanamide 17 of melting point 156° C.

¹H-NMR (CDCl₃, 270 MHz): δ(ppm)=1.3* (3H, CH₃), 2.5* (2H, CH₂), 6.95 (bs, 1H, NH), 7.65 (s, 1H, Ar—H). *very broad signals.

### 14.34,6-Dichloro-7-(4-chloro-5-trifluoromethyl-3-isothiazolyl)-2-ethyl-1,3-benzoxazole

1 ml of pyridine and 275 mg (2.7 mmol) of KHCO₃ were added to a solution of 1.2 g (2.5 mmol) of the acid amide 17 in 10 ml of dimethylformamide, and the mixture was stirred at 90° C. for 2 hours. 80 mg (0.52 mmol) of copper(I) bromide were then added, and the mixture was stirred at 140° C. for 2 hours. After cooling, the precipitate was filtered off with suction and washed with methyl tert-butyl ether, and the filtrate was concentrated under reduced pressure. The crude product was purified by column chromatography (cyclohexane/ethyl acetate=9:1). This gave 1.2 g (>100%) of slightly impure product which was purified by by MPLC. This gave 300 mg (30%) of the desired benzoxazole IDa.55.

¹H-NMR (CDCl₃, 270 MHz): δ(ppm)=1.4 (t, 3H, CH₃), 3.0 (q, 2H, CH₂), 7.55 (s, 1H, Ar—H).

### 4,6-Dichloro-7-(4-chloro-5-trifluoromethyl-3-isothiazolyl)-2-cyclopropyl-1,3-benzoxazole (Example 15; compound IDa.67)

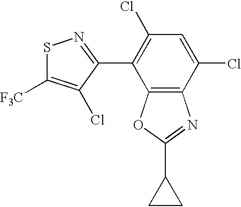

### 15.1 N-[2-Bromo-4,6-dichloro-3-(4-chloro-5-trifluoromethyl-3-isothiazolyl)phenyl]-N-(cyclopropylcarbonyl)cyclopropane-carboxamide (19) and N-[2-bromo-4,6-dichloro-3-(4-chloro-5-trifluoromethyl-3-isothiazolyl)phenyl]cyclopropane-carboxamide (18)

100 mg of dimethylaminopyridine and 390 mg (3.7 mmol) of cyclopropanecarbonyl chloride were added to a solution of 794 mg (1.80 mmol) of 2-bromo-4,6-dichloro-3-(4-chloro-5-trifluoromethyl-3-isothiazolyl)aniline 16 from Example 14.1 in 20 ml of pyridine, and the mixture was heated at 60° C. for 6 days. The mixture was concentrated under reduced pressure and the residue was then taken up in 150 ml of ethyl acetate and the organic solution was washed once with 10% strength hydrochloric acid, dried over magnesium sulfate and concentrated under reduced pressure. Column chromatography gave 630 mg (62%) of the diacylated compound 19 of melting point 110° C. and 140 mg (16%) of the monoacylated product 18 of melting point 194-196° C.

190 mg (1.1 mmol) of a 30% strength solution of sodium methoxide in methanol were added to a solution of 600 mg (1.1 mmol) of diacylated 19 in 40 ml of methanol, and the mixture was stirred at room temperature for 4 hours. Using 10% strength hydrochloric acid, the pH was then adjusted to pH 5 and the solution was concentrated under reduced pressure. Column chromatography (cyclohexane/ethyl acetate 9:1) gave 420 mg (77%) of the monoacylated product 18.

¹H-NMR (CDCl₃, 270 MHz):18: δ(ppm)=0.8 to 1.0 (m, 2H, cyclopropyl), 1.15 (m, 2H, cyclopropyl), 1.6 (m, 1H, cyclopropyl), 7.2 (s, 1H, NH), 7.65 (s, 1H, Ar—H).

¹H-NMR (CDCl₃, 270 MHz):19: δ(ppm)=0.95 (m, 4H, cyclopropyl), 1.2 (m, 4H, cyclopropyl), 2.1 (m, 2H, cyclopropyl), 7.75 (s, 1H, Ar—H).

### 15.2 4,6-Dichloro-7-(4-chloro-5-trifluoromethyl-3-isothiazolyl)-2-cyclopropyl-1,3-benzoxazole

Compound 18 was cyclized by the method described in Example 14.2, giving the title compound IDa.67 of melting point 110-111° C. in a yield of 36%.

¹H-NMR (CDCl₃, 270 MHz): δ(ppm)=1.2-1.35 (m, 4H, cyclopropyl), 2.2 (m, 1H, cyclopropyl), 7.55 (s, 1H, Ar—H).

### II Use Examples

II.1 Herbicidal Action

The herbicidal action of the 3-arylisothiazoles of the formula I according to the invention was demonstrated by greenhouse experiments:

The cultivation containers used were plastic pots containing loamy sand with approximately 3.0% of humus as the substrate. The seeds of the test plants were sewn separately for each species.

For the pre-emergence treatment, directly after sowing the active compounds, which had been suspended or emulsified in water, were applied by means of finely distributing nozzles. The containers were irrigated gently to promote germination and growth and subsequently covered with transparent plastic hoods until the plants had rooted. This cover caused uniform germination of the test plants, unless this was adversely affected by the active compounds.

For the post-emergence treatment, the test plants were first grown to a height of from 3 to 15 cm, depending on the plant habit, and only then treated with the active compounds which had been suspended or emulsified in water. The test plants were for this purpose either sown directly and grown in the same containers, or they were first grown separately as seedlings and transplanted into the test containers a few days prior to treatment. The application rate for the post-emergence treatment was 31.3 and 15.6 g of a.s./ha.

Depending on the species, the plants were kept at 10-25° C. or 20-35° C. The test period extended over 2 to 4 weeks. During this time, the plants were tended, and their response to the individual treatments was evaluated.

The evaluation was carried out using a scale from 0 to 100. 100 means no emergence of the plants, or complete destruction of at least the above-ground parts, and 0 means no damage, or normal course of growth.

The plants used in the greenhouse experiments were of the following species:

| Bayer code | Common name |
|---|---|
| ABUTH | velvet leaf |
| AMARE | redroot pigweed |
| COMBE | dayflower |
| GALAP | catchweed |
| | bedstraw |
| IPOSS | morning glory |

At application rates of 15.6 and 31.3 g of a.s./ha, the compound No. IAa.727 showed very good herbicidal action against the abovementioned harmful plants.

II.2 Desiccant/Defoliant Action

The test plants used were young cotton plants in the 4-leaf stage (calculated without cotyledons) which were grown under greenhouse conditions (rel. atmospheric humidity 50-70%, day/night temperature 27 and 20° C., respectively).

The leaves of the young cotton plants were treated to run off point with an aqueous preparation of the active compound in question which additionally contained 0.15% by weight, based on the total weight of the preparation, of a fatty alcohol ethoxylate (Plurafac® LF 700). The amount of water applied was approximately 1000 l/ha. After 13 days, the number of the leaves that had been shed and the degree of defoliation were determined. The untreated control plants showed no defoliation.

We claim:

1. A 3-arylisothiazole of the formula I 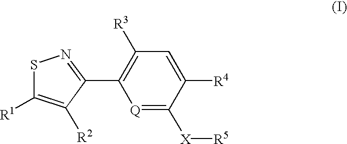
in which the variables X, Q, R¹, R², R³, R⁴, R⁵ are as defined below:
X is a chemical bond, a methylene, 1,2-ethylene, propane-1,3-diyl, ethene-1,2-diyl or ethyne-1,2-diyl chain or an oxymethylene or thiamethylene chain which is attached to the phenyl ring via the heteroatom, where all chains may be unsubstituted or may carry one or two substituents, in each case selected from the group consisting of cyano, carboxyl, halogen, C₁-C₄-alkyl, C₁-C₄-haloalkyl, C₁-C₄-alkoxy, (C₁-C₄-alkoxy)carbonyl, di(C₁-C₄-alkyl)amino and phenyl;
R¹ is C₁-C₄-haloalkyl, C₁-C₄-alkoxy, C₁-C₄-haloalkoxy, C₁-C₄-alkylthio, C₁-C₄-haloalkylthio, C₁-C₄-alkylsulfinyl, C₁-C₄-haloalkylsulfinyl, C₁-C₄-alkylsulfonyl, C₁-C₄-haloalkylsulfonyl, C₁-C₄-alkylsulfonyloxy or C₁-C₄-haloalkylsulfonyloxy;
R² is hydrogen, halogen, amino, cyano, nitro, C₁-C₄-alkyl or C₁-C₄-haloalkyl;
R³ is hydrogen or halogen;
R⁴ is hydrogen, cyano, nitro, halogen, C₁-C₄-alkyl, C₁-C₄-haloalkyl, C₁-C₄-alkoxy or C₁-C₄-haloalkoxy;
R⁵ is hydrogen, nitro, cyano, halogen, halosulfonyl, —O—Y—R⁷, —O—CO—Y—R⁷, —N(Y—R⁷)(Z—R⁸), —N(Y—R⁷)—SO₂—Z—R⁸, —N(SO₂—Y—R⁷)(SO₂—Z—R⁸), —N(Y—R⁷)—CO—Z—R⁸, —N(Y—R⁷)(O—Z—R⁸), —S—Y—R⁷, —SO—Z—R⁷, —SO₂—Y—R⁷, —SO₂—O—Y—R⁷, —SO₂—N(Y—R⁷)(Z—R⁸), —CO—Y—R⁷, —C(═NOR⁹)—Y—R⁷, C(═NOR⁹)—O—Y—R⁷, —CO—O—Y—R⁷, —CO—S—Y—R⁷, —CO—N(Y—R⁷)(Z—R⁸), —CO—N(Y—R⁷)(O—Z—R⁸) or —PO(O—Y—R⁷)₂;
Q is nitrogen or a group C—R⁶ in which R⁶ is hydrogen; or
R⁴ and X—R⁵ or X—R⁵ and R⁶ are a 3- or 4-membered chain whose chain members may, in addition to carbon, include 1, 2 or 3 heteroatoms selected from the group consisting of nitrogen, oxygen and sulfur and which may be unsubstituted or may for its part carry one, two or three substituents, and whose members may also include one or two not adjacent carbonyl, thiocarbonyl or sulfonyl groups,
where at least one of the variables R³, R⁴ and/or the group X—R⁵ is different from hydrogen and where the variables Y, Z, R⁷, R⁸ and R⁹ are as defined below:
Y, Z independently of one another are: a chemical bond, a methylene or ethylene group which may be unsubstituted or may carry one or two substituents, in each case selected from the group consisting of carboxyl, C₁-C₄-alkyl, C₁-C₄-haloalkyl, (C₁-C₄-alkoxy)carbonyl and phenyl;
R⁷, R⁸ independently of one another are: hydrogen, C₁-C₆-haloalkyl, C₁-C₄-alkoxy-C₁-C₄-alkyl, C₂-C₆-alkenyl, C₂-C₆-haloalkenyl, C₂-C₆-alkynyl, C₂-C₆-haloalkynyl, —CH(R¹⁰)(R¹¹), —C(R¹⁰)(R¹¹)—NO₂, —C(R¹⁰)(R¹¹)—CN, —C(R¹⁰)(R¹¹)-halogen, —C(R¹⁰)(R¹¹)—OR¹², —C(R¹⁰)(R¹¹)—N(R¹²)R¹³, —C(R¹⁰)(R¹¹)—N(R¹²)—OR¹³, —C(R¹⁰)(R¹¹)—SR¹², —C(R¹⁰)(R¹¹)—SO—R¹², —C(R¹⁰)(R¹¹)—SO₂—R¹², —C(R¹⁰)(R¹¹)—SO₂—OR¹², —C(R¹⁰)(R¹¹)—SO₂—N(R¹²)R¹³, —C(R¹⁰)(R¹¹)—CO—R¹², —C(R¹⁰)(R¹¹)—C(═NOR¹⁴)—R¹², —C(R¹⁰)(R¹¹)—CO—OR¹², —C(R¹⁰)(R¹¹)—CO—SR¹², —C(R¹⁰)(R¹¹)—CO—N(R¹²)R¹³, —C(R¹⁰)(R¹¹)—CO—N(R¹²)—OR¹³, —C(R¹⁰)(R¹¹)—PO(OR¹²)₂, C₃-C₈-cycloalkyl which may contain a carbonyl or thiocarbonyl ring member, phenyl or 3-, 4-, 5-, 6- or 7-membered heterocyclyl which may contain a carbonyl or thiocarbonyl ring member, where each cycloalkyl, the phenyl and each heterocyclyl ring may be unsubstituted or may carry one, two, three or four substituents, in each case selected from the group consisting of cyano, nitro, amino, hydroxyl, carboxyl, halogen, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₁-C₄-alkylsulfonyl, C₁-C₄-haloalkylsulfonyl, (C₁-C₄-alkyl)carbonyl, (C₁-C₄-haloalkyl)carbonyl, (C₁-C₄-alkyl)carbonyloxy, (C₁-C₄-haloalkyl)carbonyloxy, (C₁-C₄-alkoxy)carbonyl and di(C₁-C₄-alkyl)amino;
R⁹ is hydrogen, C₁-C₆-alkyl, C₁-C₄-alkoxycarbonyl-C₁-C₄-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, phenyl or phenyl-C₁-C₄-alkyl;
where the variables R¹⁰ to R¹⁴ are as defined below:
R¹⁰, R¹¹ independently of one another are hydrogen, C₁-C₄-alkyl, C₁-C₄-alkoxy-C₁-C₄-alkyl, C₁-C₄-alkylthio-C₁-C₄-alkyl, (C₁-C₄-alkoxy)carbonyl-C₁-C₄-alkyl or phenyl-C₁-C₄-alkyl, where the phenyl ring may be unsubstituted or may carry one to three substituents, in each case selected from the group consisting of cyano, nitro, carboxyl, halogen, C₁-C₄-alkyl, C₁-C₄-haloalkyl and (C₁-C₄-alkoxy)carbonyl;
R¹², R¹³ independently of one another are hydrogen, C₁-C₆-alkyl, C₁-C₆-haloalkyl, C₁-C₄-alkoxy-C₁-C₄-alkyl, C₂-C₆-alkenyl, C₂-C₆-haloalkenyl, C₂-C₆-alkynyl, C₂-C₆-haloalkynyl, C₃-C₈-cycloalkyl, C₃-C₈-cycloalkyl-C₁-C₄-alkyl, phenyl, phenyl-C₁-C₄-alkyl, 3- to 7-membered heterocyclyl or heterocyclyl-C₁-C₄-alkyl, where each cycloalkyl and each heterocyclyl ring may contain a carbonyl or thiocarbonyl ring member, and where each cycloalkyl, the phenyl and each heterocyclyl ring may be unsubstituted or may carry one, two, three or four substituents, in each case selected from the group consisting of cyano, nitro, amino, hydroxyl, carboxyl, halogen, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₁-C₄-alkylsulfonyl, C₁-C₄-haloalkylsulfonyl, (C₁-C₄-alkyl)carbonyl, (C₁-C₄-haloalkyl)carbonyl, (C₁-C₄-alkyl)carbonyloxy, (C₁-C₄-haloalkyl)carbonyloxy, (C₁-C₄-alkoxy)carbonyl and di(C₁-C₄-alkyl)amino;
R¹⁴ is hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, phenyl or phenyl-C₁-C₄-alkyl;
and the agriculturally useful salts of I.

2. A 3-arylisothiazole as claimed in claim 1 in which Q in formula I is nitrogen or C—H.

3. A 3-arylisothiazole as claimed in claim 2 in which R⁴ together with X—R⁵ is a chain of the formula: —O—C(R¹⁵,R¹⁶)—CO—N(R¹⁷)—, —S—C(R¹⁵,R¹⁶)—CO—N(R¹⁷)—, —N═C(R¹⁸)—O— or —N═C(R¹⁸)—S— in which the variables R¹⁵ to R¹⁸ are as defined below:
R¹⁵, R¹⁶ independently of one another are hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, phenyl or phenyl-C₁-C₄-alkyl;
R¹⁷ is hydrogen, hydroxyl, C₁-C₆-alkyl, C₁-C₆-haloalkyl, C₂-C₆-alkenyl, C₂-C₆-haloalkenyl, C₂-C₆-alkynyl, C₁-C₄-alkoxy, C₁-C₄-haloalkoxy, C₃-C₆-alkenyloxy, C₃-C₆-alkynyloxy, C₁-C₄-alkylsulfonyl, C₁-C₄-haloalkylsulfonyl, C₁-C₄-alkylcarbonyl, C₁-C₄-haloalkylcarbonyl, C₁-C₄-alkoxycarbonyl, C₁-C₄-alkoxy-C₁-C₄-alkyl, C₁-C₄-alkoxycarbonyl-C₁-C₄-alkyl, C₁-C₄-alkoxycarbonyl-C₁-C₄-alkoxy, di(C₁-C₄-alkyl)aminocarbonyl, di(C₁-C₄-alkyl)aminocarbonyl-C₁-C₄-alkyl, di(C₁-C₄-alkyl)aminocarbonyl-C₁-C₄-alkoxy, phenyl, phenyl-C₁-C₄-alkyl, C₃-C₈-cycloalkyl, C₃-C₈-cycloalkyl-C₁-C₄-alkyl, 3-, 4-, 5-, 6- or 7-membered heterocyclyl which contains one or two ring heteroatoms selected from the group consisting of oxygen, nitrogen and sulfur,
R¹⁸ is hydrogen, halogen, cyano, amino, C₁-C₆-alkyl, C₁-C₆-haloalkyl, C₂-C₆-alkenyl, C₂-C₆-haloalkenyl, C₂-C₆-alkynyl, C₁-C₄-alkoxy, C₁-C₄-haloalkoxy, C₃-C₆-alkenyloxy, C₃-C₆-alkynyloxy, C₁-C₄-alkylamino, di(C₁-C₄-alkyl)amino, C₁-C₄-haloalkoxy, C₁-C₄-alkylthio, C₁-C₄-haloalkylthio, C₁-C₄-alkylsulfinyl, C₁-C₄-haloalkylsulfinyl, C₁-C₄-alkylsulfonyl, C₁-C₄-haloalkylsulfonyl, C₁-C₄-alkylcarbonyl, C₁-C₄-haloalkylcarbonyl, C₁-C₄-alkoxy-C₁-C₄-alkyl, C₁-C₄-alkoxycarbonyl, C₁-C₄-alkoxycarbonyl-C₁-C₄-alkyl, C₁-C₄-alkoxycarbonyl-C₁-C₄-alkoxy, C₁-C₄-alkoxycarbonyl-C₁-C₄-alkylthio, di(C₁-C₄-alkyl)aminocarbonyl, di(C₁-C₄-alkyl) aminocarbonyl-C₁-C₄-alkyl, di(C₁-C₄-alkyl) aminocarbonyl-C₁-C₄-alkoxy, di(C₁-C₄-alkyl)aminocarbonyl-C₁-C₄-alkylthio, C₃-C₈-cycloalkyl, phenyl, phenyl-C₁-C₄-alkyl, C₃-C₈-cycloalkyl-C₁-C₄-alkyl, 3-, 4-, 5-, 6- or 7-membered heterocyclyl which contains one or two ring heteroatoms selected from the group consisting of oxygen, nitrogen and sulfur.

4. A 3-arylisothiazole as claimed in claim 3 in which R⁴ together with —X—R⁵ is a chain of the formula: —O—CH(R¹⁵)—CO—N(R¹⁷)— or —S—CH(R¹⁵)—CO—N(R¹⁷)—, where the nitrogen atom of the chain is attached to the carbon atom of the phenyl ring in formula I which is adjacent to the group Q.

5. A 3-arylisothiazole as claimed in claim 1 in which Q is C—R⁶ and R⁶ together with —X—R⁵ is a chain of the formula: —O—C(R¹⁵,R¹⁶)—CO—N(R¹⁷)—, —S—C(R¹⁵,R¹⁶)—CO—N(R¹⁷)—, —N═C(R¹⁸)—O—and —N═C(R¹⁸)—S— where the variables R¹⁵ to R¹⁸ are as defined in claim 3.

6. A 3-arylisothiazole as claimed in any of the preceding claims in which R¹ in formula I is selected from the group consisting of trifluoromethyl, difluoromethoxy, methylsulfonyl and methylsulfonyloxy.

7. A 3-arylisothiazole as claimed in claim 1 or 6 in which Q is CH, R² is halogen, R³ is fluorine or chlorine and R⁴ is chlorine or cyano.

8. A 3-arylisothiazole as claimed in any of the preceding claims in which R² in formula I is chlorine or bromine.

9. The use of 3-arylisothiazoles of the formula I and their agriculturally useful salts as claimed in claim 1 as herbicides or for the desiccation/defoliation of plants.

10. A composition, comprising a herbicidally effective amount of at least one 3-arylisothiazole of the formula I or an agriculturally useful salt of I as claimed in claim 1 and at least one inert liquid and/or solid carrier and, if desired, at least one surfactant.

11. A composition for the desiccation and/or defoliation of plants, comprising such an amount of at least one 3-arylisothiazole of the formula I or an agriculturally useful salt of I as claimed in claim 1 that it has desiccant and/or defoliant action and at least one inert liquid and/or solid carrier and, if desired, at least one surfactant.

12. A method for controlling undesirable vegetation, which comprises allowing a herbicidally effective amount of at least one 3-arylisothiazole of the formula I or an agriculturally useful salt of I as claimed in claim 1 to act on plants, their habitat or on seed.

13. A method for the desiccation and/or defoliation of plants, which comprises allowing such an amount of at least one 3-arylisothiazole of the formula I or an agriculturally useful salt of I as claimed in claim 1 that it has desiccant and/or defoliant action to act on plants.

14. A method as claimed in claim 13, wherein cotton is treated.

15. A process for preparing 3-arylisothiazoles of the formula I as claimed in claim 1 in which R¹ is trifluoromethyl, which comprises reacting a 3-arylisothiazole-5-carboxylic acid of the formula II 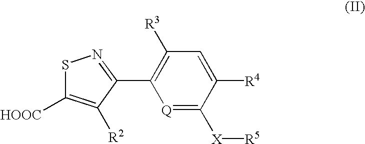
in which the variables X, Q, R², R³, R⁴, R⁵ are as defined in claim 1 with a fluorinating agent.

16. A process for preparing 7-(isothiazolyl)-1,3-benzoxazoles of the formula I-D 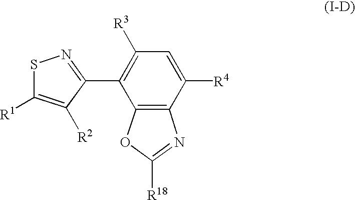
in which the variables R¹ to R⁴ and R¹⁸ are as defined in any of claims 1 to 8, which comprises reacting a 2-halo-3-(isothiazol-3-yl)anilide of the formula X, 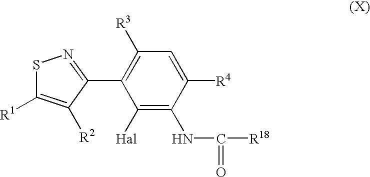
in which Hal is bromine or iodine and the variables R¹ to R⁴ and R¹⁸ are as defined above, in the presence of a transition metal compound of transition groups VIIa, VIIIa or Ib of the Periodic Table and a base to give a compound of the formula I-D.

17. A process as claimed in claim 16, wherein the transition metal compound is selected from copper, manganese, palladium, cobalt and nickel compounds.

18. A process as claimed in claim 17, wherein the transition metal compound is selected from copper(I) compounds.

19. A process as claimed in any of claims 16 to 18, wherein the molar ratio of transition metal to the compound II used is in the range from 0.05:1 to 1:1.

20. A 2-halo-3-(isothiazol-3-yl)anilide of the formula X in which the variables R¹ to R⁴, R¹⁸ and Hal are as defined above.

21. A 2-halo-3-(isothiazol-3-yl)aniline of the formula XI 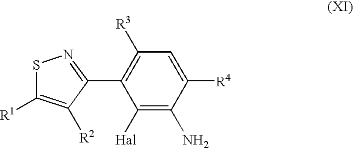
in which the variables R¹ to R⁴ R¹⁸ and Hal are as defined above.

22. An N,N-diacyl-2-halo-3-(isothiazol-3-yl)aniline of the formula XII 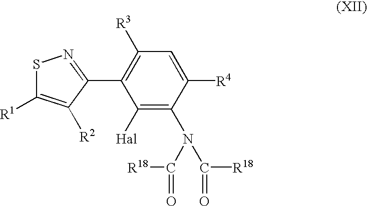
in which the variables R¹ to R⁴, R¹⁸ and Hal are as defined above.
